(19) United States
(12) Patent Application Publication
(10) Pub. No.: US 2023/0330094 A1
(43) Pub. Date: Oct. 19, 2023

(54) COMBINATION COMPOSITIONS AND METHODS FOR TREATING CANCER

(71) Applicants: The Board of Supervisors of Louisiana State University and Agricultural and Mechanical College, Baton Rouge, LA (US); AUSTRAL UNIVERSITY, BUENOS AIRES (AR)

(72) Inventors: Nicolas BAZAN, New Orleans, LA (US); Ludmila BELAYEV, New Orleans, LA (US); Juan GALLO, BUENOS AIRES (AR)

(21) Appl. No.: 18/024,928

(22) PCT Filed: Sep. 7, 2021
(86) PCT No.: PCT/US2021/049297

§ 371 Date: Mar. 6, 2023

Related U.S. Application Data
(60) Provisional application No. 63/074,968, filed on Sep. 4, 2020.

(51) Int. Cl. A61K 39/395; A61K 31/185; A61K 31/202; A61P 35/00; A61K 31/232; A61K 31/5377

(52) U.S. Cl. CPC A61K 31/5377; A61K 31/185; A61K 31/202; A61K 31/232; A61K 39/3955; A61P 35/00

(57) ABSTRACT

This invention is directed to combination compositions and methods for treating cancer, including brain cancer and its metastasis.

This application is an International Application which claims priority from U.S. Provisional Pat. Application No. 63/074,968 filed on Sep. 04, 2020, the contents of which is incorporated herein by reference in its entirety.

All patents, patent applications and publications cited herein are hereby incorporated by reference in their entirety. The disclosures of these publications in their entireties are hereby incorporated by reference into this application in order to more fully describe the state of the art as known to those skilled therein as of the date of the invention described and claimed herein.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

### GOVERNMENT INTERESTS

This invention was made with government support under Grant No. P30GM103340 awarded by the National Institutes of Health. The government has certain rights in the invention.

### FIELD OF THE INVENTION

This invention is directed to combination compositions and methods for treating cancer, including brain tumors and metastasis.

### BACKGROUND OF THE INVENTION

Glioblastoma multiforme (GBM) is the most common and aggressive primary malignant brain tumor in adults, and despite surgical resection, chemotherapy, and irradiation the average survival rate is 13-16 months. GBM is a high grade glioma originating from the glial cells of the central nervous system, accounting for 80.7% of all malignant brain tumors. The current standard of care which includes maximal surgical resection and adjuvant chemoradiation has shown little efficacy on patient survival, with median survival of less than 6 months following recurrence. The only approved medication for the treatment of newly diagnosed GBM is temozolomide (TMZ), and for recurrent GBM bevacizumab or Avastin.

### SUMMARY OF THE INVENTION

The present invention provides an anti-cancer composition and methods of use thereof. For example, aspects of the invention are drawn towards method of treating or preventing cancer, the method comprising administering to a subject a two or more anti-cancer agents, wherein the two or more anti-cancer agents are selected from the group consisting of an elovanoid, a PAF-receptor antagonist; an anti-VEGF antibody; and Suramab. In embodiments, the cancer or tumor comprises a solid tumor or a liquid cancer. In embodiments, the solid tumor comprises glioblastoma multiforme (GBM), colon cancer, prostate cancer or lung cancer. In an embodiment, wherein treating or preventing cancer is indicated by inhibiting or delaying tumor invasion, angiogenesis, tumor size, or any combination thereof. In embodiments, the two or more anti-cancer agents are administered sequentially, concurrently, or simultaneously. In embodiments, the two or more anti-cancer agents are administered systemically. In embodiments, the two or more anti-cancer agents are administered parenterally. In embodiments, the elovanoid comprises ELV-N32 and ELV-N34. In embodiments, ELV-N32 and ELV-N34 comprise

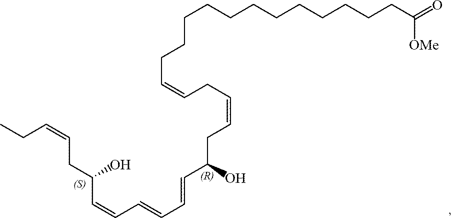

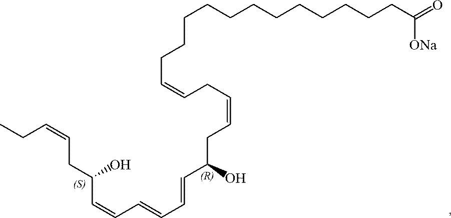

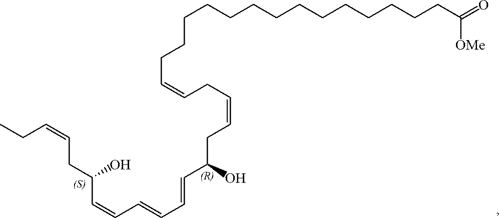

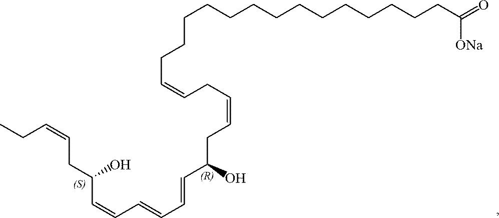

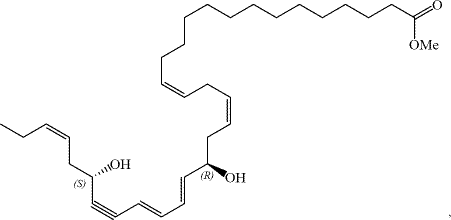

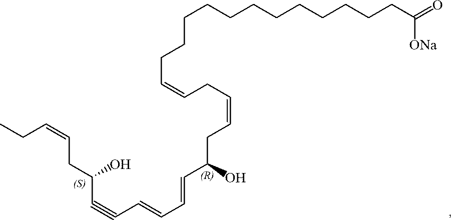

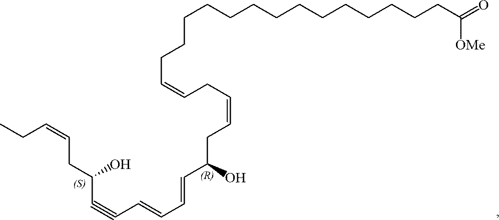

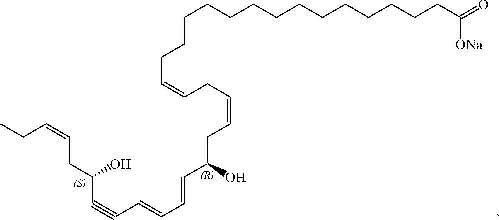

isomers thereof, or a combination thereof. In embodiments, the PAF-receptor antagonist comprises a compound according to Formula I:

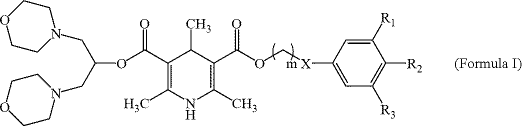

In embodiements, m is 1 - 4; X is O or S; R₁ is H or Cl; R₃ is H or Cl; R₂ is H, butoxy, or Cl; and wherein, when: R₂ is butoxy, m is 1 or 4, or when R₁ and R₂ are both Cl, and X is O, m is 3 or 4. In embodiments, the compound of Formula I comprises

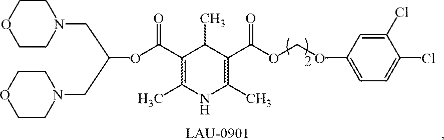

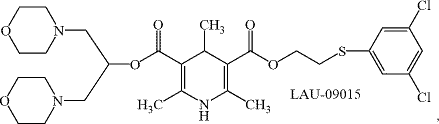

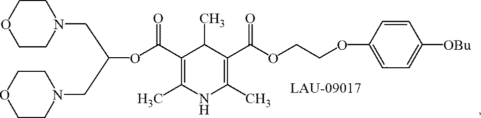

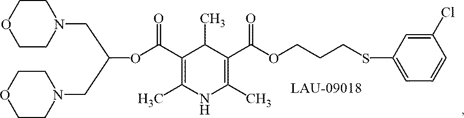

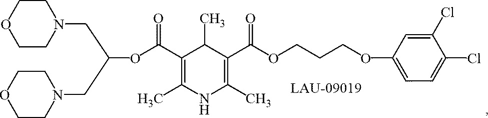

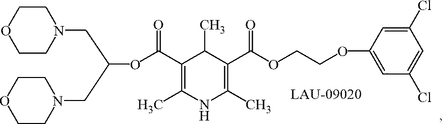

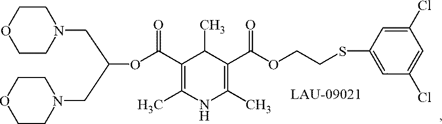

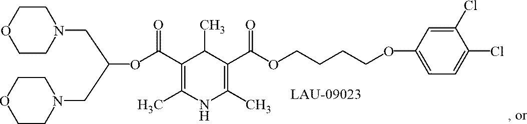

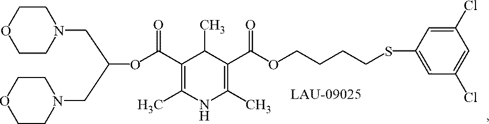

or a pharmaceutically acceptable salt thereof. In embodiments, the anti-VEGT antibody comprises bevacizumab.

Aspects of the invention are drawn towards, a method of reducing tumor size, the method comprising administering to a subject a two or more anti-cancer agents, wherein the two or more anti-cancer agents are selected from the group consisting of an elovanoid, a PAF-receptor antagonist; an anti-VEGF antibody; and Suramab. In embodiments, the cancer or tumor comprises a solid tumor or a liquid cancer. In embodiments, the solid tumor comprises glioblastoma multiforme (GBM), colon cancer, prostate cancer or lung cancer. In embodiments, the two or more anti-cancer agents are administered sequentially, concurrently, or simultaneously. In embodiments, the two or more anti-cancer agents are administered systemically. In embodiments, the two or more anti-cancer agents are administered parenterally. In embodiments, the elovanoid comprises ELV-N32 and ELV-N34. In embodiments, ELV-N32 and ELV-N34 comprise

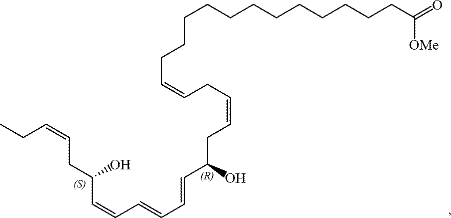

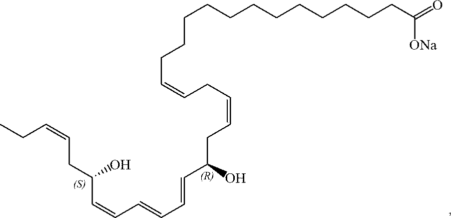

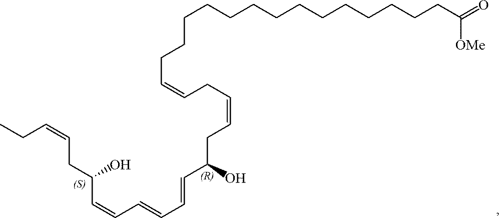

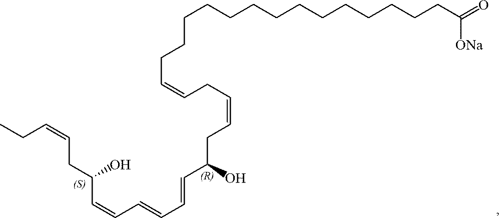

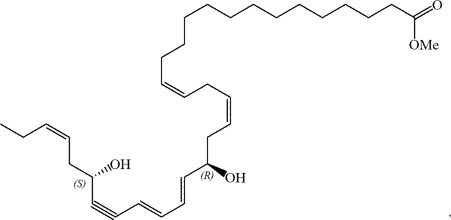

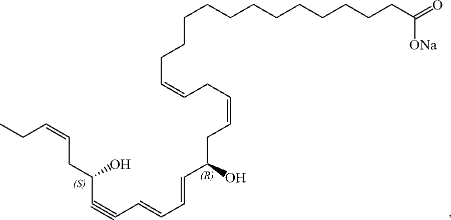

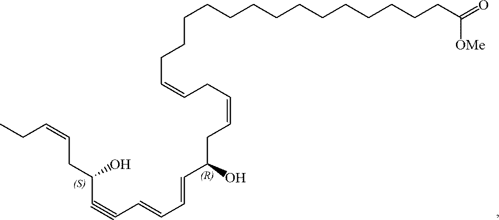

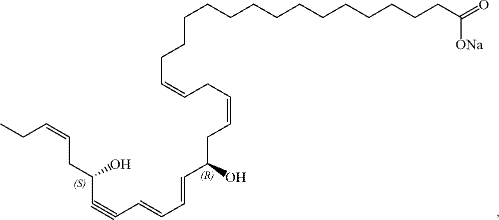

isomers thereof, or a combination thereof. In embodiments, the PAF-receptor antagonist comprises a compound according to Formula I:

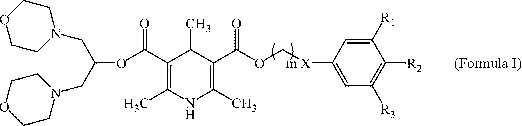

In embodiments, m is 1 - 4; X is O or S; R₁ is H or Cl; R₃ is H or Cl; R₂ is H, butoxy, or Cl; and wherein, when: R₂ is butoxy, m is 1 or 4, or when R₁ and R₂ are both Cl, and X is O, m is 3 or 4. In embodiments, the compound of Formula I comprises

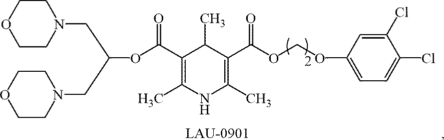

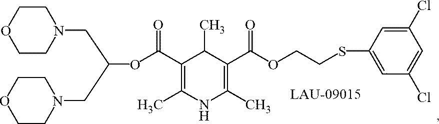

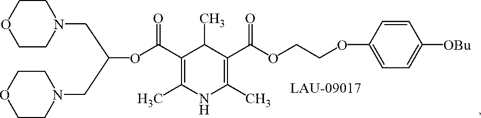

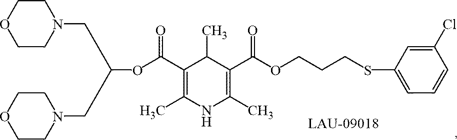

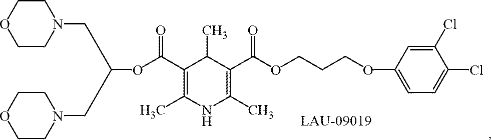

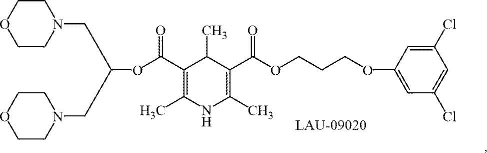

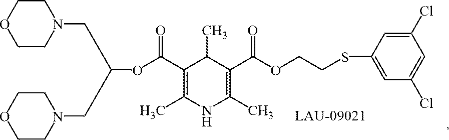

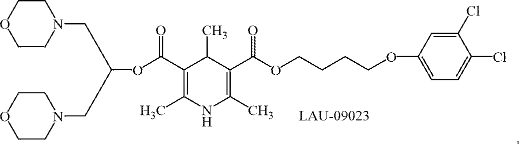

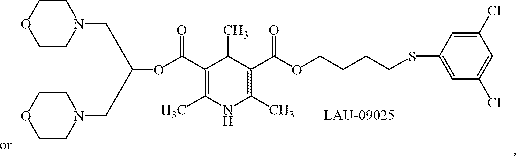

or a pharmaceutically acceptable salt thereof. In embodiments, the anti-VEGT antibody comprises bevacizumab.

Aspects of the invention are drawn towards an anti-cancer composition comprises a therapeutically effective amount of an elovanoid and a therapeutically effective amount of a PAF-receptor antagonist. In another example, the anti-cancer composition comprises a therapeutically effective amount of an elovanoid and a therapeutically effective amount of an anti-VEGF antibody. In another example, the anti-cancer composition comprises a therapeutically effective amount of an elovanoid and a therapeutically effective amount of Suramab. In another example; the anti-cancer composition comprises a therapeutically effective amount of a PAF-receptor antagonist and a therapeutically effective amount of an anti-VEGF antibody. In still another example; the anti-cancer composition comprises a therapeutically effective amount of a PAF-receptor antagonist and a therapeutically effective amount of Suramab. In embodiments, the elovanoid comprises ELV-N32 and ELV-N34. In embodiments, ELV-32 and ELVN34 comprise

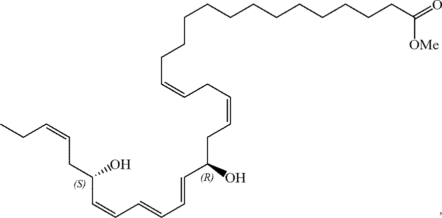

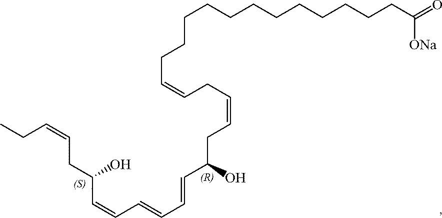

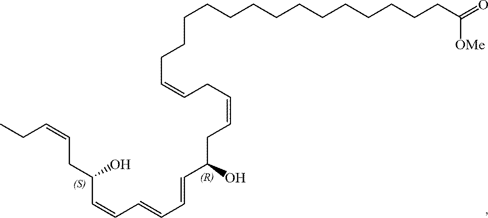

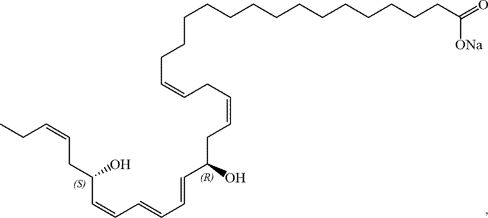

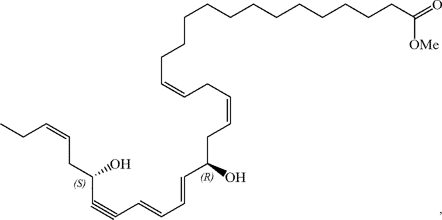

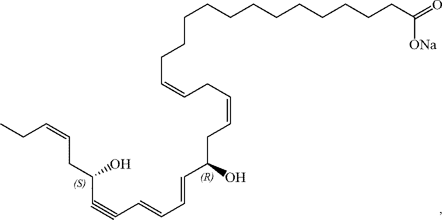

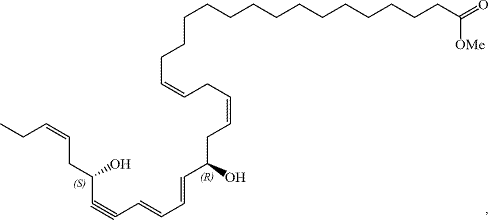

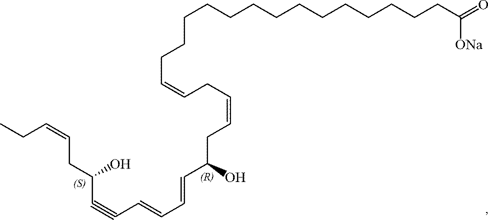

isomers thereof, or a combination thereof. In embodiements, the PAF-receptor antagonist comprises a compound of Formula I:

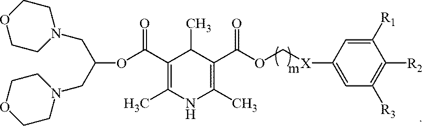

In embodiments, m is 1 - 4; X is O or S; R₁ is H or Cl; R₃ is H or Cl; R₂ is H, butoxy, or Cl; and wherein, when: R₂ is butoxy, m is 1 or 4, or when R₁ and R₂ are both Cl, and X is O, m is 3 or 4. In embodiments, the compound of Formula I comprises

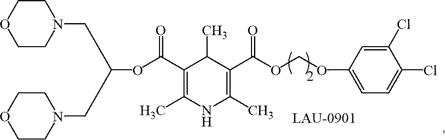

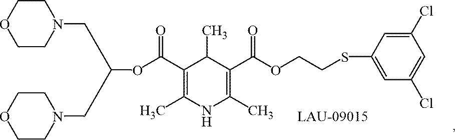

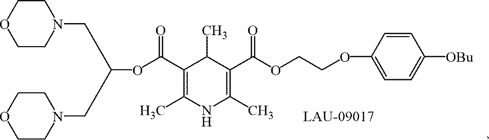

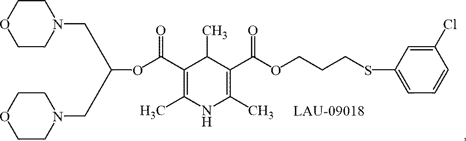

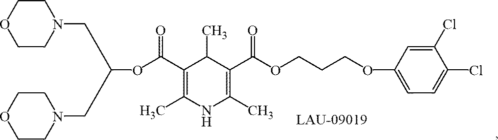

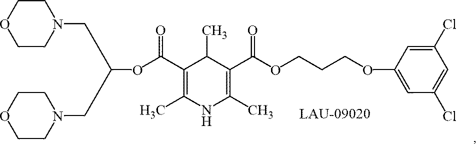

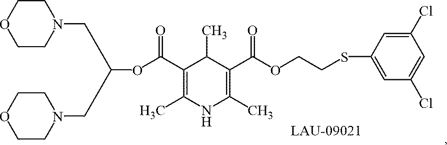

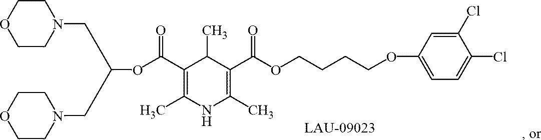

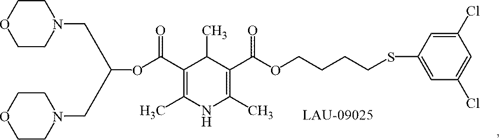

or a pharmaceutically acceptable salt thereof. In embodiments, the anti-VEGF antibody comprises bevacizumab. In embodiments, the anti-cancer composition is provided as a pharmaceutical composition. In embodiments, the pharmaceutical composition further comprises an excipient, pharmaceutically acceptable carrier, or diluent.

Asepects of the invention are drawn towards method of treating or preventing cancer, the method comprising administering to a subject a therapeutically effective amount of an anti-cancer composition comprising therapeutically effective amount of an elovanoid and a therapeutically effective amount of a PAF-receptor antagonist; a therapeutically effective amount of an elovanoid and a therapeutically effective amount of an anti-VEGF antibody; a therapeutically effective amount of an elovanoid and a therapeutically effective amount of Suramab; a therapeutically effective amount of a PAF-receptor antagonist and a therapeutically effective amount of an anti-VEGF antibody; or a therapeutically effective amount of a PAF-receptor antagonist and a therapeutically effective amount of Suramab. In embodiments, the cancer or tumor comprises a solid tumor or a liquid cancer. In embodiments, the solid tumor comprises glioblastoma multiforme (GBM), colon cancer, prostate cancer or lung cancer. In embodiments, treating cancer is indicated by inhibiting or delaying tumor invasion, angiogenesis, tumor size, or any combination thereof. In embodiments, the composition is administered systemically. In embodiments, wherein the composition is administered parenterally. In embodiments, parenteral administration comprises injection.

Aspects of the invention are drawn towards a method to reduce tumor size, the method comprising administering to a subject a therapeutically effective amount of an anti-cancer composition comprising therapeutically effective amount of an elovanoid and a therapeutically effective amount of a PAF-receptor antagonist; a therapeutically effective amount of an elovanoid and a therapeutically effective amount of an anti-VEGF antibody; a therapeutically effective amount of an elovanoid and a therapeutically effective amount of Suramab; a therapeutically effective amount of a PAF-receptor antagonist and a therapeutically effective amount of an anti-VEGF antibody; or a therapeutically effective amount of a PAF-receptor antagonist and a therapeutically effective amount of Suramab. In embodiments, the cancer or tumor comprises a solid tumor or a liquid cancer. In embodiments, the solid tumor comprises glioblastoma multiforme (GBM), colon cancer, prostate cancer or lung cancer. In embodiments, the composition is administered systemically. In embodiments, wherein the composition is administered parenterally. In embodiments, parenteral administration comprises injection.

For example, the anti-cancer composition comprises a therapeutically effective amount of an elovanoid and a therapeutically effective amount of a PAF-receptor antagonist. In another example, the anti-cancer composition comprises a therapeutically effective amount of an elovanoid and a therapeutically effective amount of an anti-VEGF antibody. In another example, the anti-cancer composition comprises a therapeutically effective amount of an elovanoid and a therapeutically effective amount of Suramab. In another example; the anti-cancer composition comprises a therapeutically effective amount of a PAF-receptor antagonist and a therapeutically effective amount of an anti-VEGF antibody. In still another example; the anti-cancer composition comprises a therapeutically effective amount of a PAF-receptor antagonist and a therapeutically effective amount of Suramab.

In embodiments, the elovanoid comprises ELV-N32 and ELV-N34.

In embodiments, the PAF-receptor antagonist comprises a compound of Formula I, for example the compound of Formula I comprises LAU-0901.

In embodiments, the anti-VEGF antibody comprises bevacizumab, ramucirumab, or ranibizumab.

In embodiments, the anti-VEGF agent comprises a small molecule VEGF inhibitor, such as azitinib, cabozantinib, lapatinib, lenvatinib, pazopanib, ponatinib, sorafenib, sunitinib, or vandetanib.

In embodiments, the anti-cancer composition is provided as a pharmaceutical composition.

In embodiments, the pharmaceutical composition further comprises an excipient, pharmaceutically acceptable carrier, or diluent.

Aspects of the invention are still further drawn towards a method of treating cancer. For example, the method comprises administering to a subject a therapeutically effective amount of the composition as described herein. For example, the cancer comprises a solid tumor or a liquid cancer. For example, the solid tumor comprises glioblastoma multiforme (GBM), colon cancer, prostate cancer or lung cancer.

In embodiments, treating cancer is indicated by inhibiting or delaying invasiveness.

In embodiments, the composition is administered systemically.

In embodiments, the composition is administered parenterally, for example parenteral administration comprises injection.

Other objects and advantages of this invention will become readily apparent from the ensuing description.

### BRIEF DESCRIPTION OF THE FIGURES

FIG. 1A shows the premise of using PAF-R antagonists in GBM. PAF-R is over-activated in the microenvironment of different tumors as part of the enhanced neuroinflammatory response. PAF-R dependent pathways have been shown to be activated during experimental tumor growth.

FIG. 1B shows athymic nude female mice were anesthetized with a ketamine/xylazine cocktail solution (100 mg/kg; 10 mg/kg). Then, mice were secured in a stereotactic head frame and a midline 1 cm incision over the scalp was made. For each mouse, 5 × 10⁶ cells in 5 µL serum free Dulbecco’s Modified Eagle Medium (DMEM) tagged with a luciferase receptor gene were implanted into the right hippocampus using a 10 µL Hamilton syringe at the following coordinates, in reference to the bregma: 1.5 mm lateral to the, 1.5 mm posterior and 3.5 mm in depth; the needle was lowered to 3.5 mm and pulled up by 1 mm, prior to the injection.

FIG. 1C shows experimental design timeline showing GBM surgery, imaging and treatments. Mice underwent stereotactic implantation of the U87GBM cells on day 0, and were monitored during a 30-day survival period. Treatment was started on day 13 post implantation. In vivo bioluminescent imaging was performed on days 13, 20 and 30 post implantation. At day 30 post implantation, mice were sacrificed, and brains were underwent ex vivo MRI.

FIG. 2A and FIG. 2B show U87GBM cells were taken out from Cryofridze, and placed in a T-25 mM flask containing 5ml DMEM-F12 (10%)-Glutamax medium and incubated at 37° C. Pictures were taken: (FIG. 2A) 3 h, (FIG. 2B) 36 h after incubation at 37° C.

FIG. 2C shows approximately 500,000 U-87GBM and human retinal pigment epithelial (h-RPE) cells allowed to grow separately in six well plates for 36 h (80% confluent) at 37° C. in triplicate. Cells washed with cold PBS, harvested and cell extracts were made. Protein contents were measured by Bio-Rad method and adjusted. Luciferase activity was measured in 5-20 µg protein equivalent extracts using Luciferin as substrate. Luciferase units (LFU) was detected in Glomax 20/20 Luminometer.

FIG. 3 shows in vivo bioluminescent imaging showing tumor progression size in all groups. Intracranial tumor growth was monitored on days 13, 20 and 30 post implantation. There was progressive and rapid tumor growth in the saline group. Mice treated with ELV, ELV+LAU-0901 and ELV+Avastin dramatically reduced tumor size.

FIG. 4 shows in vivo bioluminescent imaging growth scores (Radiance × 10⁶) for all treatment groups. All values are mean ± SD (n=5-7), *p< 0.05. All treatments significantly reduced tumor growth compared to vehicle groups.

FIG. 5A shows gadolinium (Gd)-enhanced tumor visualization on T1-weighted MRI (T1WI). Gd-DTPA was administered prior to sacrifice to enhance tumor visualization (arrows). Ex vivo T1WI of the entire cerebrum was performed to measure brain and tumor volumes. Treatment by ELV and in conjunction with LAU-0901 and Avastin, resulted in reduced GBM tumor volumes compared to saline-treated mouse as illustrated.

FIG. 5B shows all treatments significantly reduced tumor growth compared to vehicle group. All values are mean ± SD (n=5-7), *p< 0.05.

FIG. 6 shows in vivo bioluminescent imaging showing tumor progression size in all groups. Intracranial tumor growth was monitored on days 13, 20 and 30 post implantation. There was progressive and rapid tumor growth in the saline group. Mice treated with LAU-0901, Avastin and LAU-0901+Avastin reduced tumor size.

FIG. 7 shows in vivo bioluminescent imaging growth scores (Radiance × 10⁶) for all treatment groups. All values are mean ± SD (n=5-7), *p< 0.05, saline vs treatments. All treatments significantly reduced tumor growth compared to vehicle group.

FIG. 8A shows gadolinium (Gd)-enhanced tumor visualization on T1-weighted MRI (T1WI). Gd-DTPA was administered prior to sacrifice to enhance tumor visualization (arrows). Ex vivo T1WI of the entire cerebrum was performed to measure brain and tumor volumes. Treatment by Avastin, LAU-0901 and in conjunction with LAU-0901 and Avastin, resulted in reduced GBM tumor volumes compared to saline-treated mouse as illustrated.

FIG. 8B shows all treatments significantly reduced tumor growth compared to vehicle group. All values are mean ± SD (n=5-7), *p< 0.05; all treatments vs. saline.

FIG. 9 shows in vivo bioluminescent imaging growth scores (Radiance × 10⁶) for Suramab, ELV+Suramab, LAU+Suramab and vehicle treated groups. All values are mean ± SD (n=3-4). All treatments reduced tumor growth compared to vehicle group.

FIG. 10 is a scheme illustrating the biosynthesis of elovanoids (ELV) from omega-3 (n-3 or n3) very long chain polyunsaturated fatty acids (n3 VLC PUFA).

FIG. 11 is a scheme illustrating ELV-N32 and ELV-N34 synthesis from intermediates, each of which were prepared in stereochemically-pure form.

FIG. 12, panels A and B show bioluminescent data. Panel A shows bioluminescent imgaging and Panel B shows a bar graph of tumor size.

FIG. 13 shows a diagram depicting characteristics of glioblastoma and therapeutics. (Panel A) Glioblastomas comprise multiple cell types, including microglia, astrocytes, fibroblasts, and endothelial cells facilitating tumor progression. Cytokines released by glioma cells recruit immune cells into the tumor microenvironment, inducing pro-inflammatory signaling. Inflammatory signaling elicits pro-tumor activity allowing cells to evade immune cells contributing to tumor progression. Increased growth factor and platelet-activating factor (PAF) secretion from surrounding and glioma cells and their ability to evade growth factor suppressors contribute to the tumor’s proliferative and invasive nature. Vascular endothelial growth factor (VEGF) is a critical growth factor for blood vessel formation in glioblastoma.

(Panel B) Inhibition of inflammatory signaling molecules IL-1β, IL-6, and TNFα by Elovanoids (ELVs) and inhibition of growth factor and PAF activity by Suramab and LAU-0901 reduces tumor proliferation and migration. Suramab and ELV also reduce blood vessel formation through inhibition of VEGF. (Panel C) Representative bioluminescent images of the brain tumors after implantation of the luciferase modified U87MG cells from all experimental groups on day 30. The intensity of light emission corresponding to tumor burden is represented by a colorimetric scale, where red indicates the highest radiance and blue/violet shows the least. There was progressive and rapid tumor growth in the saline group. In contrast, mice treated with LAU-0901, Suramab, ELV, ELV + Suramab, and LAU-0901 + Suramab showed reduced tumor growth compared to vehicle-treated mice.

FIG. 14 shows a schematic representation of non-limiting examples of targets of LAU-0901, Elovanoids, and Avastin in the GBM tumor microenvironment. LAU-0901, a selective PAFR antagonist, prevents over-activation of PAFR. Excessive production of PAF and over activation of PAFR increases synthesis of growth factors, adhesion molecules, inflammatory signalin and promotes angiogenesis. ELVs target pro-inflammatory signaling pathways which play a role in the tumor microenvironment by inhibiting proliferation and migration of cancer cells. Avastin is a monoclonal antibody which prevents VEGF binding and, thus inhibiting angiogenesis.

FIG. 15 shows morphological growth and luciferase activity in U87MG cells. Representative images of U87MG cells at 3 (Panel A), 36 (Panel B), and 72 (Panel C) hours at 20× magnification after taking out of Cryofrizide. A luciferase receptor gene was used to tag the U87MG cells. Steady growth and attenuation of the morphological pattern of U87MG cells present at 36 and 72h. (Panel D) Detection of luciferase activity expressed in luciferase units (LFU) in U87MG and hRPE cells. Results are the average of three independent experiments.

FIG. 16 shows (Panel A) a non-limiting, exemplary experimental design, showing bregma level and site of tumor cell implantation in anesthetized athymic nude female mice, secured in a stereotactic head frame. (Panel B) Timeline showing GBM implantation, imaging, and treatments. Mice underwent stereotactic implantation of the luciferase-modified U87MG cells on day 0 and were monitored during a 30-day survival period. Treatment was started on day 13 post-implantation. In vivo bioluminescent imaging was performed on days 13, 20, and 30 post-implantation. On day 30, mice were sacrificed, and ex vivo MRI was conducted on perfused brains.

FIG. 17 shows representative bioluminescent images of the brain tumors from all experimental groups. Mice received treatment on day 13, and tumor growth progression was monitored on days 13, 20, and 30 after implantation. The intensity of light emission is indicated by a colorimetric scale, where red represents the highest amount of light emission, and blue/violet shows the least. There was progressive and rapid tumor growth in the saline group. In contrast, LAU-0901, Avastin, ELV, and combination repress orthotopic GBM.

FIG. 18 shows quantification of bioluminescent signals from tumors. Radiance (Radiance × 10⁶) values from regions of interest in mice from all groups were averaged and compared on days 13, 20, and 30 following intracranial implantation of U87-Luc cells. Tumor-bearing mice treated with (Panel A) LAU-0901, Avastin, LAU-0901 + Avastin and (Panel B) ELV, ELV + LAU-0901, and ELV + Avastin were observed have significantly reduced tumorigenesis when compared to vehicle-treated mice. All values are mean ± SEM (n =5-7), *p< 0.05.

FIG. 19 shows T1WI measurements of tumor volume. (Panel A) Representative T1-weighted images from treatment groups. Gadolinium (Gd)-enhanced tumor visualization on T1-weighted MRI (T1WI). Ex vivo T1WI of the entire cerebrum was performed to measure brain and tumor volumes. All treatments reduced intracranial tumor growth. Tumor volume was significantly reduced in LAU-0901/Avastin (Panel B) and ELV/Avastin (Panel C) compared to the vehicle group. All values are mean ± SEM (n=5-7), *p< 0.05.

### DETAILED DESCRIPTION OF THE INVENTION

Aspects of the invention are drawn to anti-cancer compounds and compositions, such as those comprising two or more anti-cancer agents. Aspects of the invention are further drawn to kits and methods for treating cancer, such as glioblastoma multiforme.

Compositions described herein can comprise, for example, two or more anti-cancer agents. Such anti-cancer agents can include therapeutic combinations of agents such as an elovanoid and a PAF-receptor antagonist, such as a LAU compound; an elovanoid and an anti-VEGF antibody; an elovanoid and suramab; a PAF-receptor antagonist and an anti-VEGF antibody; or a PAF-receptor antagonist, such as a LAU compound; and suramab. Thereapeutic combinations can comprise pharmaceutically acceptable carriers, excipients, or diluents. The combination therapy will, for example, blunt pro-angiogenic mechanisms while also block pro-inflammatory signaling, thereby treating cancer.

Detailed descriptions of one or more embodiments are provided herein. It is to be understood, however, that the t invention can be embodied in various forms. Therefore, specific details disclosed herein are not to be interpreted as limiting, but rather as a basis for the claims and as a representative basis for teaching one skilled in the art to employ the present invention in any appropriate manner.

The singular forms “a”, “an” and “the” include plural reference unless the context clearly dictates otherwise. The use of the word “a” or “an” when used in conjunction with the term “comprising” in the claims and/or the specification may mean “one,” but it is also consistent with the meaning of “one or more,” “at least one,” and “one or more than one.”

Wherever any of the phrases “for example,” “such as,” “including” and the like are used herein, the phrase “and without limitation” is understood to follow unless explicitly stated otherwise. Similarly, “an example,” “exemplary” and the like are understood to be nonlimiting.

The term “substantially” allows for deviations from the descriptor that do not negatively impact the intended purpose. Descriptive terms are understood to be modified by the term “substantially” even if the word “substantially” is not explicitly recited.

The terms “comprising” and “including” and “having” and “involving” (and similarly “comprises”, “includes,” “has,” and “involves”) and the like are used interchangeably and have the same meaning. Specifically, each of the terms is defined consistent with the common United States patent law definition of “comprising” and is therefore interpreted to be an open term meaning “at least the following,” and is also interpreted not to exclude additional features, limitations, aspects, etc. Thus, for example, “a process involving steps a, b, and c” means that the process includes at least steps a, b and c. Wherever the terms “a” or “an” are used, “one or more” is understood, unless such interpretation is nonsensical in context.

As used herein the term “about” is used herein to mean approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of 20 percent up or down (higher or lower).

### Anti-Cancer Compostions and Combinations

Aspects of the invention are directed towards anti-cancer compounds and compositions. Anti-cancer compounds described herein include anti-VEGF agents; pharmaceutical compounds, such as Suramab; prohomeostatic lipid mediators; and/or PAF-receptor antagonists. For example, the anti-cancer compounds can comprise an elovanoid; a PAF-receptor antagonist, such as a LAU compound; an anti-VEGF antibody, such as avastin; and/or suramab. The anti-cancer compounds can be included in therapeutic combinations comprising two or more anti-cancer compounds and a pharmaceutically acceptable carrier, diluent, or excipient.

The term “anti-cancer” can refer to an action of suppressing the growth of cancer cells or killing cancer cells and an action of suppressing or blocking metastasis of cancer cells in connection with prevention and treatment of cancer. For example, anti-cancer can refer to inhibition of formation, infiltration, metastasis, and growth of cancers.

Anti-cancer compounds described herein can comprise anti-VEGF agents. The term “anti-VEGF agent” can refer to an agent or compound which inhibits, either partially or completely, the function, activity or effect of vascular endothelial growth factor (VEGF), and includes, for example, a VEGF inhibitor, a VEGF receptor inhibitor, and/or a nucleic acid that inhibits expression of VEGF. Anti-VEGF agents can include, for example, antibodies and fragments thereof. For example, Bevacizumab (also referred to as Avastin) is a recombinant humanized monoclonal antibody that blocks angiogenesis by inhibiting VEGF-A. VEGF-A is a growth factor protein that stimulates angiogenesis in a variety of diseases, such as cancer. Bevacizumab binds VEGF and prevents the interaction of VEGF to its receptors (Flt-1 and KDR) on the surface of endothelial cells. The interaction of VEGF with its receptors leads to endothelial cell proliferation and new blood vessel formation in in vitro models of angiogenesis. Administration of bevacizumab to xenotransplant models of colon cancer in nude (athymic) mice caused reduction of microvascular growth and inhibition of metastatic disease progression. Bevacizumab has been used to treat colon cancer, lung cancer, renal cancer, ovarian cancer, and glioblastoma multiforme, among others. As another example, Ranibizumab (also known as Lucentis) is a monoclonal antibody fragment (Fab) created from the same parent moust antibody as bevacizumab. Other anti-VEGF antibodies include, but are not limited to, ramucirumab or ranibizumab. In embodiments, the anti-VEGF agent can refer to monoclonal antibodies, polyclonal antibodies, or fragments thereof.

In embodiments, the anti-VEGF agent can also be a small molecule VEGF inhibitor, non-limiting examples of which comprise azitinib, cabozantinib, lapatinib, lenvatinib, pazopanib, ponatinib, sorafenib, sunitinib, and vandetanib. The term “small molecule inhibitor” can refer to a compound having a measurable VEGF-inhibiting activity.

Anti-cancer compounds as described herein can comprise anti-angiogenic agents. An anti-angiogenic agent can refer to a compound that interferes with, or to some extent, the development of blood vessels.

Anti-cancer compounds of the invention can include pharmaceutical compounds and compositions, for example, Suramab. Suramab is a compound of Bevacizumab, described herein, and Suramin. Suramab has been shown to have anti-angiogenic and anti-neoplastic properties. See, for example, US 9,023,350B2, which is incorporated herein by reference in its entirety. Suramin is a drug known for many years for the treatment of diseases caused by nematodes and protozoa. For example, Suramin is a medication used to treat African sleeping sickness and river blindness. Its properties have also been described as an anti-neoplastic agent through several mechanisms: inhibition of tumor angiogenesis, increase in cellular sensitivity to cytotoxic substances (chemotherapy) and cytotoxic action itself. The molecular formula of Surmin is C₅₁H₄₀N₆O₂₃S₆ and CAS number 145-63-1.

Anti-cancer compounds of the invention can also include pro-homeostatic lipid mediators, such as elovanoids and derivatives thereof. As used herein, the term “pro-homeostatic” can refer to the ability to promote or maintain homeostasis or a homeostatic state. As used herein, the pharse “lipid mediator” can refer to a class of biologically active lipids which can be produced via biosynthesis in response to extracellular stimuli. See, for example, WO2016130522A1 and WO2018175288A1, each of which are incorporated by reference herein in their entireties. n-3 very-long-chain polyunsaturated fatty acids (“n-3 VLC-PUFA”, also “n3 VLC-PUFA”) are converted in vivo to several previously unknown types of VLC-PUFA hydroxylated derivatives named elovanoids (ELVs) that can protect and prevent the progressive damage to tissues and organs, whose functional integrity has been disrupted. In embodiments, the term “derivative” can refer to a structural analog. In embodiments, the term “derivative” can refer to a compound derived from a similar compound by a chemical reaction. ELVs have structures resembling docosanoids but with different physicochemical properties and alternatively-regulated biosynthetic pathways. In embodiments, for example, the elovanoids comprise 32- and/or 34- carbon elovanoids termed ELV-N32 and ELV-N34, or salts thereof.

The mono-hydroxylated elovanoids of the disclosure can have the structures of G, H, I or J:

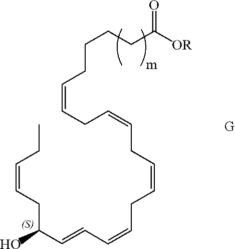

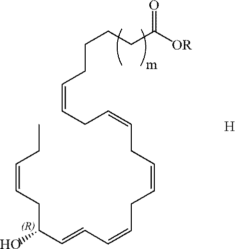

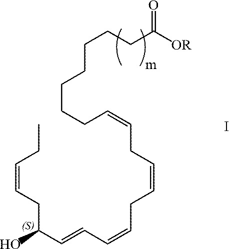

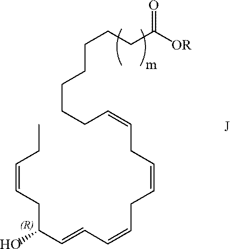

wherein compounds G and H can have a total from 23 to 42 carbon atoms in the carbon chain, with 5 cis carbon-carbon double bonds starting at positions n-3, n-9, n-12, n-15 and n-18 and a trans carbon-carbon double bond starting at positions n-7; and wherein compounds I and J can have a total from 23 to 42 carbon atoms in the carbon chain, and with 4 cis carbon-carbon double bonds starting at positions n-3, n-9, n-12 and n-15, and a trans carbon-carbon double bond starting at positions n-7; wherein R is hydrogen, methyl, ethyl, alkyl, or a cation selectedfrom a group consisting of: ammonium cation, iminium cation, or a metal cation selected from a group consisting of sodium, potassium, magnesium, zinc, or calcium cation, and wherein m is a number selected from a group consisting of 0 to 19; wherein compounds G and H can exist as an equimolar mixture; wherein compounds I and J can exist as an equimolar mixture; wherein, the provided compounds G and H can be predominately one enantiomer with a defined (S) or (R) chirality at the carbon bearing the hydroxyl group; and wherein, the compounds G and H can be predominately one enantiomer with a defined (S) or (R) chirality at the carbon bearing the hydroxyl group.

As used herein and in other structures of the disclosure, the compounds of the disclosure are shown having a terminal carboxyl group “—COOR” the “R” can be a group covalently bonded to the carboxyl such as an alkyl group. In the alternative, the carboxyl group can further have a negative charge as “—COO⁻” and R is a cation including a metal cation, an ammonium cation and the like.

In some embodiments of the mono-hydroxylated elovanoids of the disclosure, m is a number selected from a group consisting of 0 to 15. In other embodiments, m is a number selected from 1, 3, 5, 7, 9, 11, 13, or 15 where the fatty acid component contains a total of 24, 26, 28, 30, 32, 34, 36 or 38 carbon atoms in its carbon chain. In other embodiments, m is a number selected from a group consisting of 0, 2, 4, 6, 8, 10, 12 or 14, where the fatty acid component contains a total of 23, 25, 27, 19, 31, 33, 35 or 37 carbon atoms in its carbon chain.

In some embodiments, m is a number selected from a group consisting of 5 to 15, where the fatty acid component contains a total of 28, 29, 30, 31, 32, 33, 34, 35, 36, 37 or 38 carbon atoms in its carbon chain. In some embodiments, m is a number selected from a group consisting of 5, 7, 9, 11, 13, or 15, where the fatty acid component contains a total of 28, 30, 32, 34, 36 or 38 carbon atoms in its carbon chain. In other embodiments, m is a number selected from a group consisting of 4, 6, 8, 10, 12 or 14, where the fatty acid component contains a total of 27, 29, 31, 33, 35 or 37 carbon atoms in its carbon chain. In embodiments, m is a number selected from a group consisting of 9 to 11, where the fatty acid component contains a total of 32 or 34 carbon atoms in its carbon chain.

In some embodiments the mono-hydroxylated elovanoids of the disclosure are a carboxylic acid, i.e., R is hydrogen. In other embodiments the compound is a carboxylic ester, wherein R is methyl, ethyl or alkyl. In embodiments the compound is a carboxylic ester, wherein R is methyl or ethyl. In embodiments the compound is a carboxylic ester, wherein R is methyl. In other embodiments the compound is a carboxylate salt, wherein R is an ammonium cation, iminium cation, or a metal cation selected from a group consisting of sodium, potassium, magnesium, zinc, or calcium cation. In some embodiments, R is ammonium cation or iminium cation. In other embodiments, R is a sodium cation or a potassium cation. In embodiments, R is a sodium cation.

The di-hydroxylated elovanoids of the disclosure can have the structures K, L, M, or N

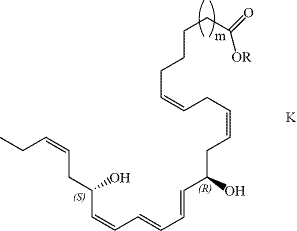

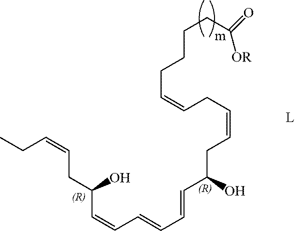

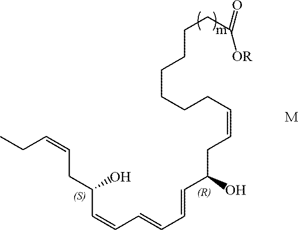

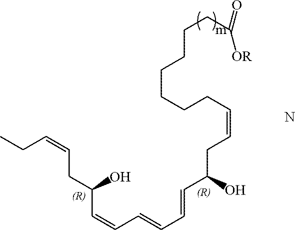

wherein compounds K and L can have a total from 23 to 42 carbon atoms in the carbon chain, with 4 cis carbon-carbon double bonds starting at positions n-3, n-7, n-15 and n-18, and 2 trans carbon-carbon bonds starting at positions n-9, n-11; and wherein compounds M and N can have a total from 23 to 42 carbon atoms in the carbon chain, with 3 cis carbon-carbon double bonds starting at positions n-3, n-7, n-12 and n-15, and 2 trans carbon-carbon bonds starting at positions n-9, n-11, wherein R is hydrogen, methyl, ethyl, alkyl, or a cation selected from a group consisting of: ammonium cation, iminium cation, or a metal cation selected from a groupconsisting of sodium, potassium, magnesium, zinc, or calcium cation, and wherein m is a number selected from a group consisting of 0 to 19; wherein compounds K and L can exist as an equimolar mixture; wherein compounds M and N can exist as an equimolar mixture, wherein the compounds K and L can be predominately one enantiomer with a defined (S) or (R) chirality at the carbon bearing the hydroxyl group; and wherein, the provided compounds M and N can be predominately one enantiomer with a defined (S) or (R) chirality at the carbon bearing the hydroxyl group.

As used herein and in other structures of the present disclosure, the compounds of the disclosure are shown having a terminal carboxyl group “—COOR” the “R” can be a group covalently bonded to the carboxyl such as an alkyl group. In the alternative, the carboxyl group can further have a negative charge as “—COO⁻” and R is a cation including a metal cation, an ammonium cation and the like.

In some embodiments of the di-hydroxylated elovanoids of the disclosure, m is a number selected from a group consisting of 5 to 15, where the fatty acid component contains a total of 28, 29, 30, 31, 32, 33, 34, 35, 36, 37 or 38 carbon atoms in its carbon chain. In advantageous embodiments, m is a number selected from a group consisting of 5, 7, 9, 11, 13, or 15, where the fatty acid component contains a total of 28, 30, 32, 34, 36 or 38 carbon atoms in its carbon chain. In other embodiments, m is a number selected from a group consisting of 4, 6, 8, 10, 12 or 14, where the fatty acid component contains a total of 27, 29, 31, 33, 35 or 37 carbon atoms in its carbon chain. In embodiments, m is a number selected from a group consisting of 9 to 11, where the fatty acid component contains a total of 32 or 34 carbon atoms in its carbon chain.

Some di-hydroxylated elovanoids of the disclosure are carboxylic acid, i.e., R is hydrogen. In other embodiments the di-hydroxylated elovanoid of the disclosure is a carboxylic ester, wherein R is methyl, ethyl or alkyl. In embodiments the compound is a carboxylic ester, wherein R is methyl or ethyl. In advantageous embodiments the compound is a carboxylic ester, wherein R is methyl.

In other embodiments the di-hydroxylated elovanoid of the disclosure is a carboxylate salt, wherein R is an ammonium cation, iminium cation, or a metal cation selected from a group consisting of sodium, potassium, magnesium, zinc, or calcium cation. In some advantageous embodiments, R is ammonium cation or iminium cation. In other embodiments, R is a sodium cation or a potassium cation. In advantageous embodiments, R is a sodium cation.

The alkynyl mono-hydroxylated elovanoids of the disclosure can have the structures of O, P, Q or R:

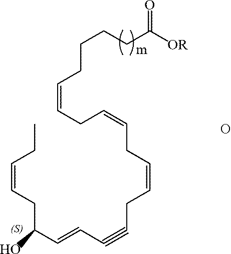

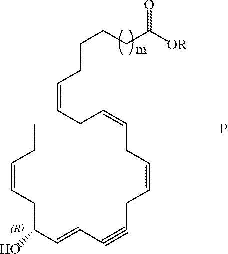

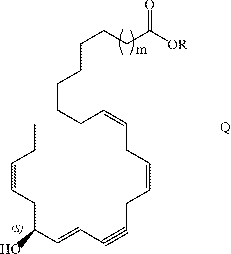

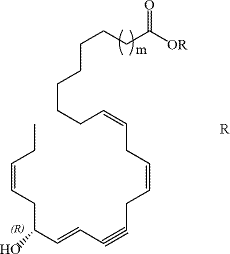

wherein compounds O and P can have a total from 23 to 42 carbon atoms in the carbon chain, with 4 cis carbon-carbon double bonds starting at positions n-3, n-12, n-15 and n-18, a trans carbon-carbon bond starting at position n-7, and a carbon-carbon triple bond starting at position n-9; and wherein compounds I and J can have a total from 23 to 42 carbon atoms in the carbon chain, with 3 cis carbon-carbon double bonds starting at positions n-3, n-12 and n-15, a trans carbon-carbon bond starting at position n-7, and a carbon-carbon triple bond starting at position n-9; wherein R is hydrogen, methyl, ethyl, alkyl, or a cation selected from a group consisting of: ammonium cation, iminium cation, or a metal cation selected from a group consisting of sodium, potassium, magnesium, zinc, or calcium cation, and wherein m is a number selected from a group consisting of 0 to 19; wherein compounds O and P can exist as an equimolar mixture; wherein compounds Q and R can exist as an equimolar mixture; wherein, the provided compounds O and P can be predominately one enantiomer with a defined (S) or (R) chiralityat the carbon bearing the hydroxyl group; and wherein, the provided compounds O and P can be predominately one enantiomer with a defined (S) or (R) chirality at the carbon bearing the hydroxyl group.

As used herein and in other structures of the present invention, the alkynyl mono-hydroxylated elovanoids of the disclosure are shown having a terminal carboxyl group “—COOR” the “R” can be a group covalently bonded to the carboxyl such as an alkyl group. In the alternative, the carboxyl group can further have a negative charge as “—COO⁻” and R is a cation including a metal cation, an ammonium cation and the like.

In some embodiments, m is a number selected from a group consisting of 0 to 15. In other embodiments, m is a number selected from 1, 3, 5, 7, 9, 11, 13, or 15 where the fatty acid component contains a total of 24, 26, 28, 30, 32, 34, 36 or 38 carbon atoms in its carbon chain.

In additional embodiments, m is a number selected from a group consisting of 0, 2, 4, 6, 8, 10, 12 or 14, where the fatty acid component contains a total of 23, 25, 27, 19, 31, 33, 35 or 37 carbon atoms in its carbon chain. In some embodiments, m is a number selected from a group consisting of 5 to 15, where the fatty acid component contains a total of 28, 29, 30, 31, 32, 33, 34, 35, 36, 37 or 38 carbon atoms in its carbon chain. In embodiments, m is a number selected from a group consisting of 5, 7, 9, 11, 13, or 15, where the fatty acid component contains a total of 28, 30, 32, 34, 36 or 38 carbon atoms in its carbon chain. In other embodiments, m is a number selected from a group consisting of 4, 6, 8, 10, 12 or 14, where the fatty acid component contains a total of 27, 29, 31, 33, 35 or 37 carbon atoms in its carbon chain. In some embodiments, m is a number selected from a group consisting of 9 to 11, where the fatty acid component contains a total of 32 or 34 carbon atoms in its carbon chain.

In some embodiments the alkynyl mono-hydroxylated elovanoids of the disclosure are carboxylic acids, for example R is hydrogen. In other embodiments the alkynyl mono-hydroxylated elovanoids of the disclosure are carboxylic esters, wherein R is methyl, ethyl or alkyl. In embodiments the alkynyl mono-hydroxylated elovanoids of the disclosure are carboxylic esters, wherein R is methyl or ethyl.

In some embodiments R is methyl. In other embodiments, alkynyl mono-hydroxylated elovanoids of the disclosure can be a carboxylate salt, wherein R is an ammonium cation, iminium cation, or a metal cation selected from a group consisting of sodium, potassium, magnesium, zinc, or calcium cation. In some embodiments, R is ammonium cation or iminium cation. In other embodiments, R is a sodium cation or a potassium cation. In embodiments, R is a sodium cation.

The alkynyl di-hydroxylated elovanoids can have the structures of S, T, U or V:

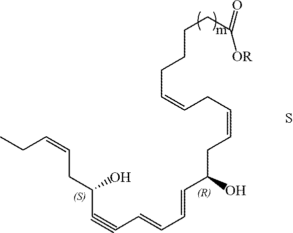

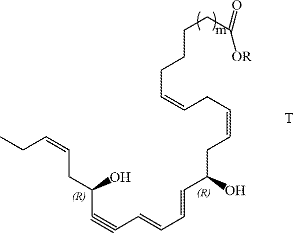

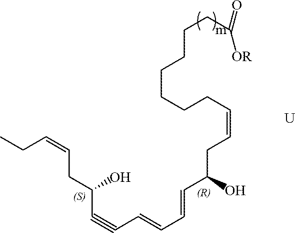

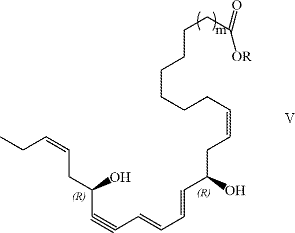

wherein compounds S and T have a total from 23 to 42 carbon atoms in the carbon chain, with 3 cis carbon-carbon double bonds starting at positions n-3, n-12, n-15 and n-18, with 2 trans carbon-carbon double bonds starting at positions n-9 and n-11, and a carbon-carbon triple bond starting at position n-7; and wherein compounds U and V have a total from 23 to 42 carbon atoms in the carbon chain, and with 2 cis carbon-carbon double bonds starting at positions n-3 and n-15, with 2 trans carbon-carbon double bonds starting at positions n-9 and n-11, and a carbon-carbon triple bond starting at position n-7; wherein R is hydrogen, methyl, ethyl, alkyl, or a cation selected from a group consisting of: ammonium cation, iminium cation, or a metal cation selected from a group consisting of sodium, potassium, magnesium, zinc, or calcium cation, and wherein m is a number selected from a group consisting of 0 to 19; wherein compounds S and T can exist as an equimolar mixture; wherein compounds U and V can exist as an equimolar mixture.

In some embodiments, the provided compounds S and T are predominately one enantiomer with a defined (S) or (R) chirality at the carbon bearing the hydroxyl group; and wherein, the provided compounds U and V are predominately one enantiomer with a defined (S) or (R) chirality at the carbon bearing the hydroxyl group.

As used herein and in other structures described herein, the compounds of the invention are shown having a terminal carboxyl group “—COOR” the “R” can be a group covalently bonded to the carboxyl such as an alkyl group. In the alternative, the carboxyl group can further have a negative charge as “—COO⁻” and R is a cation including a metal cation, an ammonium cation and the like.

In some embodiments, m is a number selected from a group consisting of 5 to 15, where the fatty acid component contains a total of 28, 29, 30, 31, 32, 33, 34, 35, 36, 37 or 38 carbon atoms in its carbon chain. In embodiments, m is a number selected from a group consisting of 5, 7, 9, 11, 13, or 15, where the fatty acid component contains a total of 28, 30, 32, 34, 36 or 38 carbon atoms in its carbon chain. In other embodiments, m is a number selected from a group consisting of 4, 6, 8, 10, 12 or 14, where the fatty acid component contains a total of 27, 29, 31, 33, 35 or 37 carbon atoms in its carbon chain. In embodiments, m is a number selected from a group consisting of 9 to 11, where the fatty acid component contains a total of 32 or 34 carbon atoms in its carbon chain.

In some embodiments the provided compound is a carboxylic acid, for example R is hydrogen.

In other embodiments the provided compound is a carboxylic ester, wherein R is methyl, ethyl or alkyl. In embodiments the provided compound is a carboxylic ester, wherein R is methyl or ethyl. In embodiments the provided compound is a carboxylic ester, wherein R is methyl. In other embodiments the provided compound is a carboxylate salt, wherein R is an ammonium cation, iminium cation, or a metal cation selected from a group consisting of sodium, potassium, magnesium, zinc, or calcium cation. In some embodiments, R is ammonium cation or iminium cation. In other embodiments, R is a sodium cation or a potassium cation. In embodiments, R is a sodium cation.

Embodiments described herein comprises a mono-hydroxylated 32-carbon methyl ester of formula G1, having the name: methyl (S,14Z,17Z,20Z,23Z,25E,29Z)-27-hydroxydotriaconta-14,17,20,23,25,29-hexaenoate; a mono-hydroxylated 32-carbon sodium salt of formula G2, having the name: sodium (S,14Z,17Z,20Z,23Z,25E,29Z)-27-hydroxydotriaconta-14,17,20,23,25,29-hexaenoate; a mono-hydroxylated 34-carbon methyl ester of formula G3, having the name: methyl (S,16Z,19Z,22Z,25Z,27E,31Z)-29-hydroxytetratriaconta-16,19,22,25,27,31-hexaenoate; or a mono-hydroxylated 34-carbon sodium salt of formula G4, having the name sodium (S,16Z,19Z,22Z,25Z,27E,31Z)-29-hydroxytetratriaconta-16,19,22,25,27,31-hexaenoate:

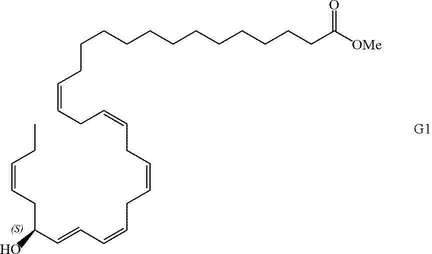 [SMILES: CC/C=C\C[C@H](O)/C=C/C=C\C/C=C\C/C=C\C/C=C\CCCCCCCCCCCCC(=O)OC]

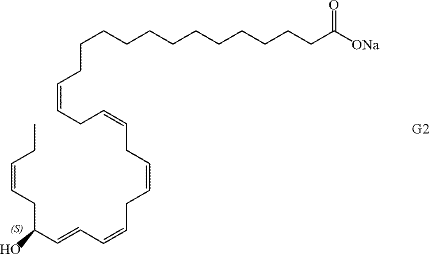

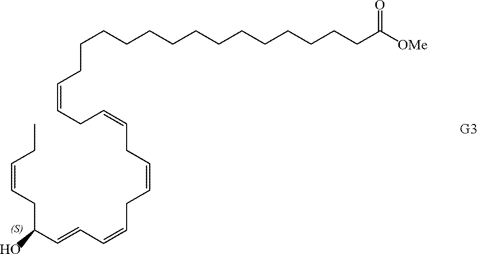 [SMILES: CC/C=C\C[C@H](O)/C=C/C=C\C/C=C\C/C=C\C/C=C\CCCCCCCCCCCCCCC(=O)OC]

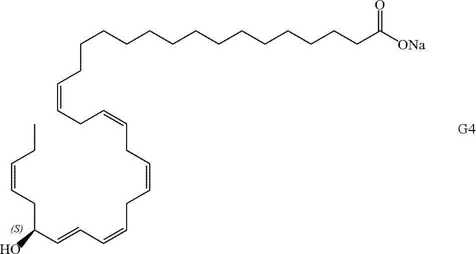

Embodiments described herein also comprises a di-hydroxylated 32-carbon methyl ester of formula K1, having the name: methyl (14Z,17Z,20R,2IE,23E,25Z,27S,29Z)-20,27-dihydroxydotriaconta-14,17,21,23,25,29-hexaenoate; a di-hydroxylated 32-carbon sodium salt of formula K2, having the name: sodium (14Z,17Z,20R,21E,23E,25Z,27S,29Z)-20,27-dihydroxydotriaconta-14,17,21,23,25,29-hexaenoate; or a di-hydroxylated 34-carbon methyl ester of formula K3, having the name: methyl (16Z,19Z,22R,23E,25E,27Z,29S,31Z)-22,29-dihydroxytetratriaconta-16,19,23,25,27,31-hexaenoate ; or a di-hydroxylated 34-carbon sodium salt of formula K4, having the name: sodium (16Z,19Z,22R,23E,25E,27Z,29S,31Z)-22,29-dihydroxytetratriaconta-16,19,23,25,27,31-hexaenoate:

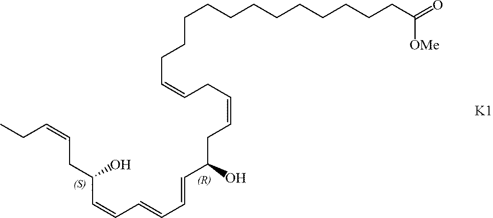 [SMILES: CC/C=C\C[C@H](O)\C=C/C=C/C=C/[C@H](O)C/C=C\C/C=C\CCCCCCCCCCCCC(=O)OC]

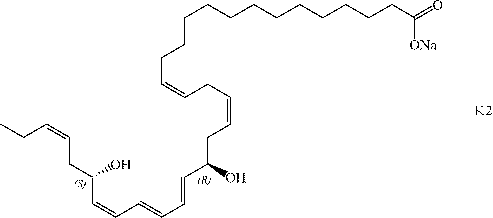

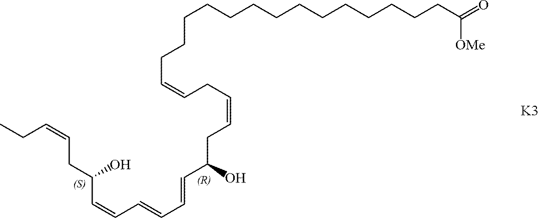 [SMILES: CC/C=C\C[C@H](O)\C=C/C=C/C=C/[C@H](O)C/C=C\C/C=C\CCCCCCCCCCCCCCC(=O)OC]

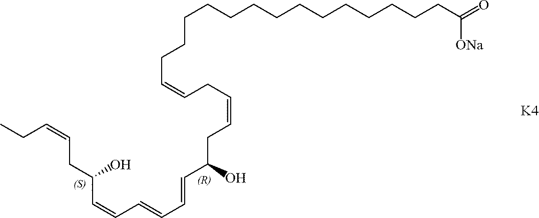

Other embodiments provide an alkynyl mono-hydroxylated 32-carbon methyl ester of formula O1, having the name: methyl (S,14Z,17Z,20Z,25E,29Z)-27-hydroxydotriaconta-14,17,20,25,29-pentaen-23-ynoate; an alkynyl mono-hydroxylated 32-carbon sodium salt of formula O2, having the name: sodium (S,17Z,20Z,25E,29Z)-27-hydroxydotriaconta-17,20,25,29-tetraen-23-ynoate; an alkynyl mono-hydroxylated 34-carbon methyl ester of formula O3, having the name: methyl (S,16Z,19Z,22Z,27E,31Z)-29-hydroxytetratriaconta-16,19,22,27,31-pentaen-25-ynoate; an alkynyl mono-hydroxylated 34-carbon sodium salt of formula 04, having the name: sodium (S,16Z,19Z,22Z,27E,31Z)-29-hydroxytetratriaconta-16,19,22,27,31-pentaen-25-ynoate:

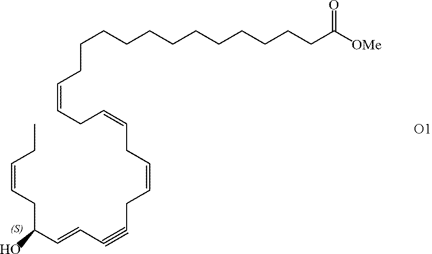 [SMILES: CC/C=C\C[C@H](O)/C=C/C#CC/C=C\C/C=C\C/C=C\CCCCCCCCCCCCC(=O)OC]

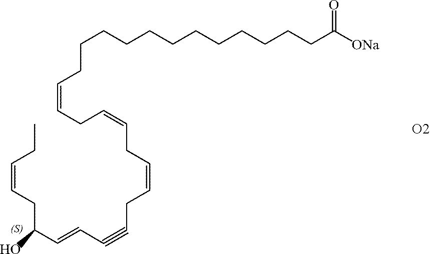

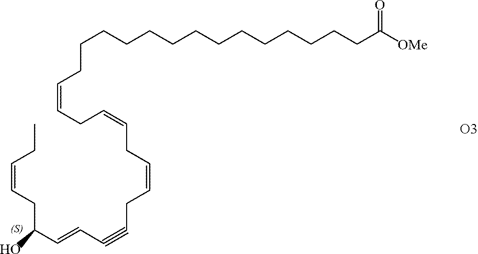 [SMILES: CC/C=C\C[C@H](O)/C=C/C#CC/C=C\C/C=C\C/C=C\CCCCCCCCCCCCCCC(=O)OC]

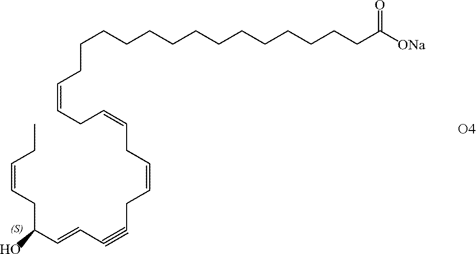

Still other embodiments provide an alkynyl di-hydroxylated 32-carbon methyl ester of formula S1, having the name: methyl (14Z,17Z,20R,21E,23E,27S,29Z)-20,27-dihydroxydotriaconta-14,17,21,23,29-pentaen-25-ynoate; an alkynyl di-hydroxylated 32-carbon sodium salt of formula S2, having the name: sodium (14Z,17Z,20R,21E,23E,27S,29Z)-20,27-dihydroxydotriaconta-14,17,21,23,29-pentaen-25-ynoate; or an alkynyl di-hydroxylated 34-carbon methyl ester of formula S3, having the name: methyl (16Z,19Z,22R,23E,25E,29S,31Z)-22,29-dihydroxytetratriaconta-16,19,23,25,31-pentaen-27-ynoate ; or an alkynyl di-hydroxylated 34-carbon sodium salt of formula S4, having the name: sodium (16Z,19Z,22R,23E,25E,29S,31Z)-22,29-dihydroxytetratriaconta-16,19,23,25,31-pentaen-27-ynoate.

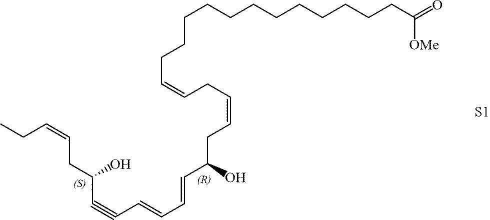 [SMILES: CC/C=C\C[C@H](O)C#C/C=C/C=C/[C@H](O)C/C=C\C/C=C\CCCCCCCCCCCCC(=O)OC]

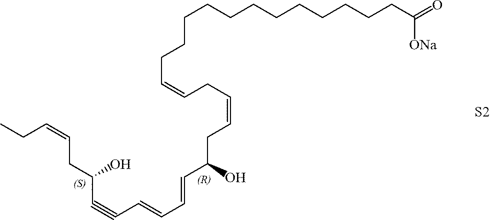

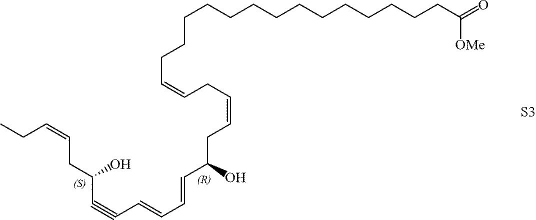 [SMILES: CC/C=C\C[C@H](O)C#C/C=C/C=C/[C@H](O)C/C=C\C/C=C\CCCCCCCCCCCCCCC(=O)OC]

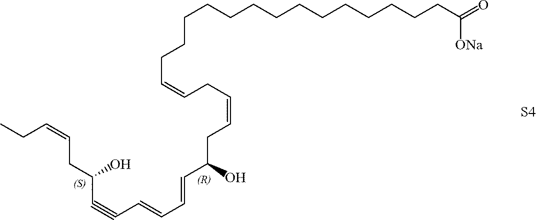

Aspects of the invention are drawn towards enantiomers and diasteromers of the compounds described herein. For example, if an (R, R) compound is described herein, aspects of the invention can also be drawn towards, for example, (S, S), (R, S), and (S, R).

Anti-cancer compounds of the invention can further include Platelet-Activating Factor-receptor antagonists. See, for example, U.S. Pat. 6,566,359 and U.S. Pat. Application 15/556,716, each of which are incorporated herein by reference in their entireties.

The term “Platelet-Activating Factor receptor” (PAF-r) can refer to a G-protein coupled receptor that shows structural characteristics of the rhodopsin gene family and binds platelet-activating factor. PAF is a phospholipid (1-0-alkyl-2-acetyl-sn-glycero-3-phosphorylcholine) that has been implicated as a mediator in diverse pathologic processes, such as allergy, asthma, septic shock, arterial thrombosis, and inflammatory processes.

The term “Platelet-Activating Factor”, interchangeably known as PAF, PAF-acether or AGEPC (acetyl glyceryl-ether-phosphorylcholine), is a potent phospholipid activator and mediator of many leukocyte functions, platelet aggregation and degranulation, inflammation, and anaphylaxis. It is also involved in changes to vascular permeability, the oxidative burst, chemotaxis of leukocytes, as well as augmentation of arachidonic acid metabolism in phagocytes. PAF is produced by a variety of cells, but especially those involved in host defense, such as platelets, endothelial cells, neutrophils, monocytes, and macrophages. PAF is produced by these cells but in low quantities and production is controlled by the activity of PAF acetyl hydrolases (PAF-AH). It is produced in larger quantities by inflammatory cells in response to specific stimuli. See, for example, Bazan, Nicolas G. “A signal terminator.” Nature 374.6522 (1995): 501-502.

The PAF signaling system can trigger inflammatory and thrombotic cascades, amplify these cascades when acting with other mediators, and mediates molecular and cellular interactions (cross talk) between inflammation and thrombosis. Unregulated PAF signaling can cause pathological inflammation and has been found to be a cause in sepsis, shock, and traumatic injury. PAF can be used as a local signaling molecule and travel over very short distances or it can be circulated throughout the body. PAF also induces apoptosis that is independent of the PAF receptor. The pathway to apoptosis can be inhibited by negative feedback from PAF acetylhydrolase (PAF - AH) that catabolizes platelet-activating factor.

Several molecular species of platelet - activating factor that vary in the length of the O-alkyl side-chain have been identified. Its alkyl group is connected by an ether linkage at the C1 carbon to a 16-carbon chain. The acyl group at the C2 carbon is an acetate unit whose short length increases the solubility of PAF allowing it to function as a soluble signal messenger. The C3 has a phosphocholine head group, just like standard phosphatidylcholine.

PAF cannot be modified without losing its biological activity. Thus, small changes in the structure of PAF can render its signaling abilities inert. Platelet and blood pressure response are dependent on the sn-2 propionyl analog. If the sn-1 is removed than PAF lacks biological activity. Finally, at the sn-3 position of PAF as an increasing number of methyl groups are removed sequentially, biological activity diminishes until inactivated.

PAF antagonists are a type of receptor ligand or drug that does not provoke an inflammatory response upon binding, but blocks or lessens the effect of PAF. Examples of PAF antagonists include, but are not limited to, such as CV-3988, a PAF antagonist that blocks signaling events correlated to the expression and binding of PAF to the PAF receptor, SM-12502, a PAF antagonist that is metabolized in the liver by the enzyme CYP2A6, and Rupatadine, an antihistamine and PAF antagonist used to treat allergies.

A PAF-r antagonist compound termed LAU-09021, 2,4,6-trimethyl-1,4-dihidropyridine derivative, and having the structure below is described herein:

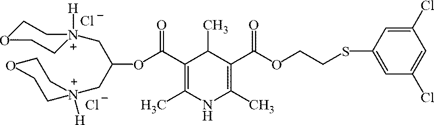 [SMILES: CC1=C(C(=O)OCCSc2cc(Cl)cc(Cl)c2)C(C)C(C(=O)OC(C[NH+]2CCOCC2)C[NH+]2CCOCC2)=C(C)N1 ; [Cl-] ; [Cl-]]

The compounds of the disclosure can be, but not exclusively, racemic mixtures, for example a 50% of S-LAU-09021 and another 50% of R-LAU-09021. However, the chiral resolution and separation of R- and S- enantiomers of the compounds of the disclosure can be isolated by methods well known in the art. Accordingly, one aspect of the disclosure encompasses embodiments of a composition comprising at least one compound having the Formula I, or a pharmaceutically acceptable salt thereof

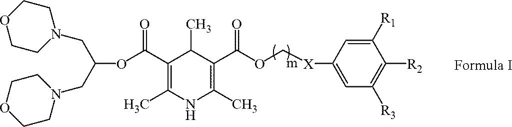

wherein: m is 1 - 4, X is O or S, R₁, and R₃ are independently H or Cl, R₂ is H, butoxy, or Cl, and wherein, when: R₂ is butoxy, m is 1 or 4, or when R₁, and R₂, are both Cl, and X is O, m is 3 or 4.

In some embodiments of this aspect of the disclosure, the at least one compound having the formula I can be selected from the group consisting of:

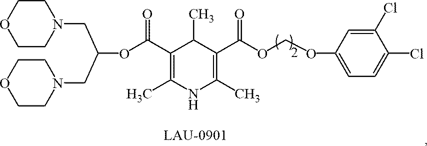

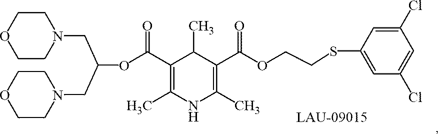

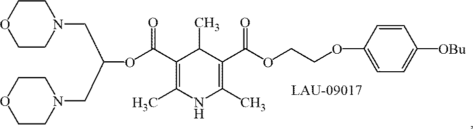

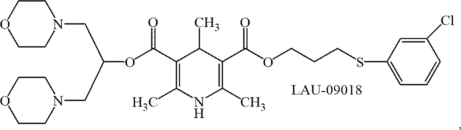

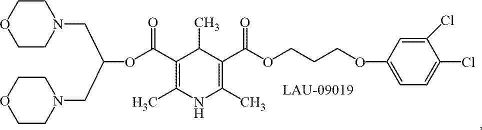

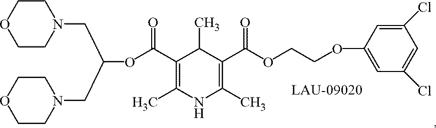

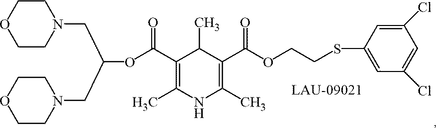

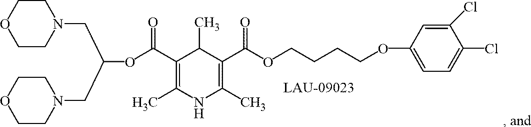

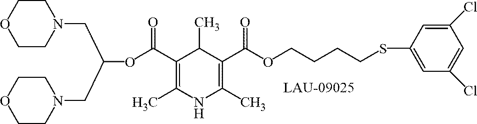

or a pharmaceutically acceptable salt thereof.

In some embodiments of this aspect of the disclosure, the compound can be an R-enantimomer, an S-enantimomer, or a combination thereof.

The anti-cancer agent or pharmaceutical composition comprising the same can be provided in an amount effective to inhibit the growth of a tumor, such as a brain tumor. Such an amount can be referred to as a “therapeutically effective amount”. The term “therapeutically effective amount” can refer to that amount of an anti-cancer agent or pharmaceutical composition being administered that will relieve to some extent one or more of the symptoms of the disease or condition being treated, and/or that amount that will prevent, or that will prevent to some extent, one or more of the symptoms of the condition or disease that the subject being treated has or is at risk of developing. In an embodiment, therapeutically effective amount can refer to an amount needed to treat cancer, such as a brain tumor, or at least one pathological effect resulting from the presence of a cancerous condition in a subject human or animal.

In embodiments a therapeutically effective amount of PAF-receptor antagonist can comprise less than about 0.1 mg/kg, about 0.1 mg/kg, about 0.5 mg/kg, about 1.0 mg/kg, about 2.5 mg/kg, about 5 mg/kg, about 7.5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 70 mg/kg, about 80 mg/kg, about 90 mg/kg, about 100 mg/kg, about 120 mg/kg, about 135 mg/kg, about 150 mg/kg, about 175 mg/kg, about 200 mg/kg, about 225 mg/kg, about 250 mg/kg, about 275 mg/kg, about 300 mg/kg, about 325 mg/kg, about 350 mg/kg, about 375 mg/kg, about 400 mg/kg, about 425 mg/kg, about 450 mg/kg, about 475 mg/kg, about 500 mg/kg, about 525 mg/kg, about 550 mg/kg, about 575 mg/kg, about 600 mg/kg, about 625 mg/kg, about 650 mg/kg, about 675 mg/kg, about 700 mg/kg, about 725 mg/kg, about 750 mg/kg, about 775 mg/kg, about 800 mg/kg, about 825 mg/kg, about 850 mg/kg, about 875 mg/kg, about 900 mg/kg, about 1.0 g/kg, about 1.5 g/kg, about 2.0 g/kg, about 2.5 g/kg, about 5 g/kg, about 10 g/kg, about 25 g/kg, about 50 g/kg, or more than 50 g/kg of compound per body weight of a subject. For example, the PAF-r antagonist can comprise LAU-0901. For example, the PAF-r antagonist can comprise LAU-0901, LAU-09015, LAU-09017, LAU-09017, LA-09018, LAU-09019, LAU-09020, LAU-09021, LAU-09023, or LAU-09025. For example, a non-limiting example of a therapeutic amount can can comprise about 30 mg/kg per a subject’s body weight.

In embodiments, a therapeutically effective amount of elovanoid can comprise less than 0.01 mg/kg, about 0.01 mg/kg, about 0.05 mg/kg, about 0.1 mg/kg, about 0.1 mg/kg, about 0.5 mg/kg, about 1.0 mg/kg, about 2.5 mg/kg, about 5 mg/kg, about 7.5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 70 mg/kg, about 80 mg/kg, about 90 mg/kg, about 100 mg/kg, about 120 mg/kg, about 135 mg/kg, about 150 mg/kg, about 175 mg/kg, about 200 mg/kg, about 225 mg/kg, about 250 mg/kg, about 275 mg/kg, about 300 mg/kg, about 325 mg/kg, about 350 mg/kg, about 375 mg/kg, about 400 mg/kg, about 425 mg/kg, about 450 mg/kg, about 475 mg/kg, about 500 mg/kg, about 525 mg/kg, about 550 mg/kg, about 575 mg/kg, about 600 mg/kg, about 625 mg/kg, about 650 mg/kg, about 675 mg/kg, about 700 mg/kg, about 725 mg/kg, about 750 mg/kg, about 775 mg/kg, about 800 mg/kg, about 825 mg/kg, about 850 mg/kg, about 875 mg/kg, about 900 mg/kg, about 1.0 g/kg, about 1.5 g/kg, about 2.0 g/kg, about 2.5 g/kg, about 5 g/kg, about 10 g/kg, about 25 g/kg, about 50 g/kg, or more than 50 g/kg of compound per body weight of a subject. For example, the elovanoid can comprise ELV-32 or ELV-34. For example, ELV-32 and ELV-34 can comprise

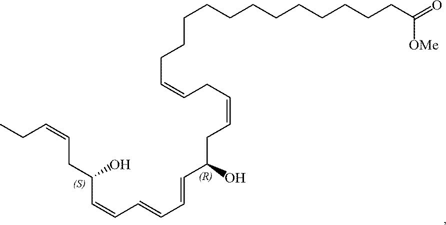

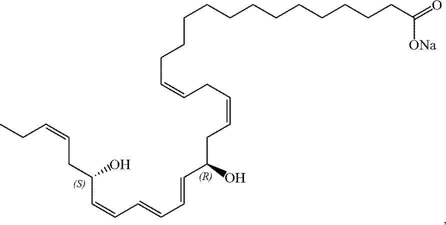

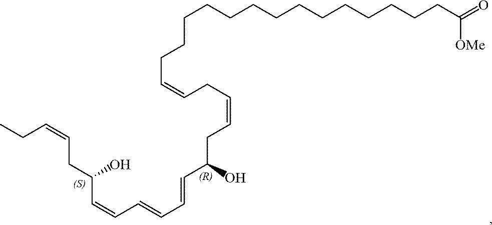

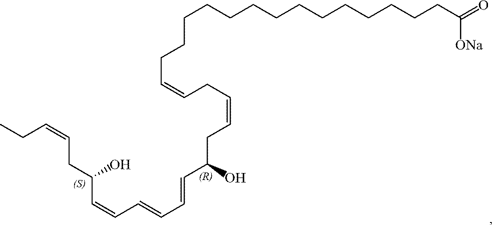

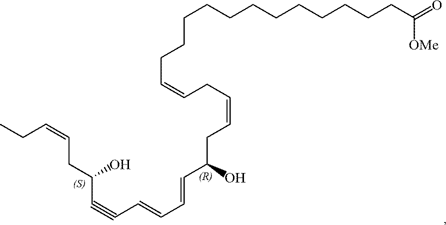

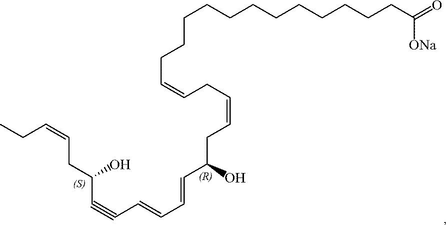

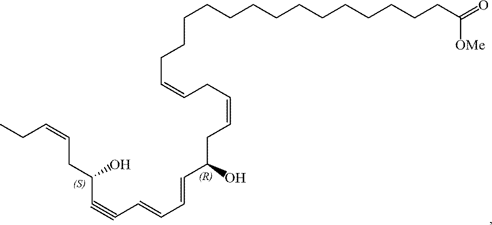

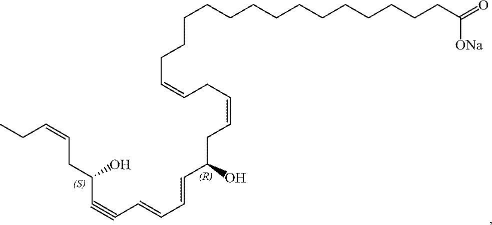

an isomer thereof, or a combination thereof. For example, a non-limiting example of a therapeutic amount can comprise about 1.62 mg/kg per body weight of a subject.

In embodiments, a therapeutically effective amount of anti-VEGF antibody can comprise less than 0.1 mg/kg, about 0.1 mg/kg, about 0.5 mg/kg, about 1.0 mg/kg, about 2.5 mg/kg, about 5 mg/kg, about 7.5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 70 mg/kg, about 80 mg/kg, about 90 mg/kg, about 100 mg/kg, about 120 mg/kg, about 135 mg/kg, about 150 mg/kg, about 175 mg/kg, about 200 mg/kg, about 225 mg/kg, about 250 mg/kg, about 275 mg/kg, about 300 mg/kg, about 325 mg/kg, about 350 mg/kg, about 375 mg/kg, about 400 mg/kg, about 425 mg/kg, about 450 mg/kg, about 475 mg/kg, about 500 mg/kg, about 525 mg/kg, about 550 mg/kg, about 575 mg/kg, about 600 mg/kg, about 625 mg/kg, about 650 mg/kg, about 675 mg/kg, about 700 mg/kg, about 725 mg/kg, about 750 mg/kg, about 775 mg/kg, about 800 mg/kg, about 825 mg/kg, about 850 mg/kg, about 875 mg/kg, about 900 mg/kg, about 1.0 g/kg, about 1.5 g/kg, about 2.0 g/kg, about 2.5 g/kg, about 5 g/kg, about 10 g/kg, about 25 g/kg, about 50 g/kg, or more than 50 g/kg of compound per body weight of a subject. For example, the anti-VEGF antibody can comprise bevacizumab. As used herein, bevacizumab and Avastin can be used interchangeably. Non-limiting examples of a therapeutic amount can comprise about 10. 8 mg/kg or about 3 mg/kg per body weight of a subject.

In embodiments, a therapeutically effective amount of a non-selective P2 purinegic antagonist can comprise less than 0.1 mg/kg, about 0.1 mg/kg, about 0.5 mg/kg, about 1.0 mg/kg, about 2.5 mg/kg, about 5 mg/kg, about 7.5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 70 mg/kg, about 80 mg/kg, about 90 mg/kg, about 100 mg/kg, about 120 mg/kg, about 135 mg/kg, about 150 mg/kg, about 175 mg/kg, about 200 mg/kg, about 225 mg/kg, about 250 mg/kg, about 275 mg/kg, about 300 mg/kg, about 325 mg/kg, about 350 mg/kg, about 375 mg/kg, about 400 mg/kg, about 425 mg/kg, about 450 mg/kg, about 475 mg/kg, about 500 mg/kg, about 525 mg/kg, about 550 mg/kg, about 575 mg/kg, about 600 mg/kg, about 625 mg/kg, about 650 mg/kg, about 675 mg/kg, about 700 mg/kg, about 725 mg/kg, about 750 mg/kg, about 775 mg/kg, about 800 mg/kg, about 825 mg/kg, about 850 mg/kg, about 875 mg/kg, about 900 mg/kg, about 1.0 g/kg, about 1.5 g/kg, about 2.0 g/kg, about 2.5 g/kg, about 5 g/kg, about 10 g/kg, about 25 g/kg, about 50 g/kg, or more than 50 g/kg of compound per body weight of a subject. For example, the non-selective P2 purinegic antagonist is suramin. A non-limiting example of a therapeutic amount can comprise about 3 mg/kg per a subject’s body weight.

In embodiments, a therapeutically effective amount of Suramab can comprise 0.1 mg/kg, about 0.1 mg/kg, about 0.5 mg/kg, about 1.0 mg/kg, about 2.5 mg/kg, about 5 mg/kg, about 7.5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 70 mg/kg, about 80 mg/kg, about 90 mg/kg, about 100 mg/kg, about 120 mg/kg, about 135 mg/kg, about 150 mg/kg, about 175 mg/kg, about 200 mg/kg, about 225 mg/kg, about 250 mg/kg, about 275 mg/kg, about 300 mg/kg, about 325 mg/kg, about 350 mg/kg, about 375 mg/kg, about 400 mg/kg, about 425 mg/kg, about 450 mg/kg, about 475 mg/kg, about 500 mg/kg, about 525 mg/kg, about 550 mg/kg, about 575 mg/kg, about 600 mg/kg, about 625 mg/kg, about 650 mg/kg, about 675 mg/kg, about 700 mg/kg, about 725 mg/kg, about 750 mg/kg, about 775 mg/kg, about 800 mg/kg, about 825 mg/kg, about 850 mg/kg, about 875 mg/kg, about 900 mg/kg, about 1.0 g/kg, about 1.5 g/kg, about 2.0 g/kg, about 2.5 g/kg, about 5 g/kg, about 10 g/kg, about 25 g/kg, about 50 g/kg, or more than 50 g/kg of compound per body weight of a subject.

As used interchangeably herein, “subject,” “individual,” or “patient,” can refer to a vertebrate, such as a mammal, for example a human. Mammals can include, but are not limited to, murines, simians, humans, farm animals, sport animals, and pets. The term “pet” includes a dog, cat, guinea pig, mouse, rat, rabbit, ferret, and the like. The term “farm animal” includes a horse, sheep, goat, chicken, pig, cow, donkey, llama, alpaca, turkey, and the like.

As used herein, a “pharmaceutical composition” or a “pharmaceutical formulation” can refer to a composition or pharmaceutical composition for administration to a subject, such as a mammal, especially a human and that can refer to the combination of one or more anti-cancer agents described herein with a pharmaceutically acceptable carrier or excipient, making the composition suitable for diagnostic, therapeutic, or preventive use in vitro, in vivo, or ex vivo. A “pharmaceutical composition” can be sterile, and can be free of contaminants that can elicit an undesirable response within the subject (for example, the compound(s) in the pharmaceutical composition is pharmaceutical grade). Pharmaceutical compositions can be designed for administration to subjects or patients in need thereof via a number of different routes of administration including oral, intravenous, buccal, rectal, parenteral, intraperitoneal, intradermal, intratracheal, intramuscular, subcutaneous, inhalational and the like.

Pharmaceutical compositions can further comprise an excipient, pharmaceutically acceptable carrier, or diluent. A “pharmaceutically acceptable excipient,” “pharmaceutically acceptable diluent,” “pharmaceutically acceptable carrier,” or “pharmaceutically acceptable adjuvant” can refer to an excipient, diluent, carrier, and/or adjuvant that is useful in preparing a pharmaceutical composition that is safe, non-toxic and neither biologically nor otherwise undesirable, and can include an excipient, diluent, carrier, and adjuvant that is acceptable for veterinary use and/or human pharmaceutical use. “A pharmaceutically acceptable excipient, diluent, carrier and/or adjuvant” as used in the specification and claims can include one and more such excipients, diluents, carriers, and adjuvants.

The compositions or pharmaceutical compositions can be formulated with one or more pharmaceutically acceptable excipients, diluents, carriers, and/or adjuvants to provide an embodiment of a composition of the disclosure. A wide variety of pharmaceutically acceptable excipients are known in the art. Pharmaceutically acceptable excipients have been amply described in a variety of publications, including, for example, A. Gennaro (2000) “Remington: The Science and Practice of Pharmacy, ” 20th edition, Lippincott, Williams, & Wilkins; Pharmaceutical Dosage Forms and Drug Delivery Systems (1999) H. C. Ansel et al., eds., 7th ed., Lippincott, Williams, & Wilkins; and Handbook of Pharmaceutical Excipients (2000) A.H. Kibbe et al., eds., 3rd ed. Amer. Pharmaceutical Assoc.

The compositions or pharmaceutical compositions of the disclosure can be administered to the subject using any means that can result in the desired effect. Thus, the composition or pharmaceutical composition can be incorporated into a variety of formulations for therapeutic administration. For example, the composition or pharmaceutical composition can be formulated into pharmaceutical compositions by combination with appropriate, pharmaceutically acceptable carriers or diluents, and can be formulated into preparations in solid, semi-solid, liquid or gaseousforms, such as tablets, capsules, powders, granules, ointments, solutions, suppositories, injections, inhalants and aerosols.

In pharmaceutical dosage forms, the composition or pharmaceutical composition can be administered in the form of its pharmaceutically acceptable salts. Pharmaceutically acceptable salts can include, but are not limited to, amine salts, such as but not limited to N,N′-dibenzylethylenediamine, chloroprocaine, choline, ammonia, diethanolamine and other hydroxyalkylamines, ethylenediamine, N-methylglucamine, procaine, N-benzylphenethylamine, 1-para-chlorobenzyl-2-pyrrolidin-1′-ylmethylbenzimidazole, diethylamineand other alkylamines, piperazine and tris(hydroxymethyl) aminomethane; alkali metal salts, such as but not limited to lithium, potassium and sodium; alkali earth metal salts, such as but not limited to barium, calcium and magnesium; transition metal salts, such as but not limited to zinc; and other metal salts, such as but not limited to sodium hydrogen phosphate and disodium phosphate; and also including, but not limited to, salts of mineral acids, such as but not limited to hydrochlorides and sulfates; and salts of organic acids, such as but not limited to acetates, lactates, malates, tartrates, citrates, ascorbates, succinates, butyrates, valerates and fumarates.

A pharmaceutically active composition can be used alone or in appropriate association, as well as in combination, with other pharmaceutically active compounds.

A reference to a compound of the disclosure and sub-groups thereof also includes ionic forms, salts, solvates, isomers, tautomers, esters, prodrugs, isotopes and protected forms thereof; such as, the salts or tautomers or isomers or solvates thereof; and more advantageously, the salts or tautomers or solvates thereof. As used herein, the term “isomer” can refer to molecules or polyamtoic ions with identical molecular formulas, but distinct arrangements of atoms in space. For example, constitutional isomers and stereoisomers of compounds described herein are also embodiments of the invention. For example, the enantiomers and diasteromers of compounds described herein can be aspects of the invention. For example, if (R,S) of a compound is described herein, (R, R), (S, R), and (S, S) can also be aspects of the invention.. As used herein, the term “enantiomer” can refer to molecules which are nonsuperimposable mirror images of each other. As used herein, the term “diastereomer” can refer to a stereoisomer of a compound having two or more chiral senters that is not a mirror image of another stereoisomer of the same compound.

Many compounds of those described herein, for example, can exist in the form of salts, for example acid addition salts or, in certain cases salts of organic and inorganic bases such as phenolate, carboxylate, sulphonate and phosphate salts. All such salts are within the scope of this disclosure.

The salts of the present disclosure can be synthesized from the parent compound that contains a basic or acidic moiety by conventional chemical methods such as methods described in Pharmaceutical Salts: Properties. Selection, and Use, P. Heinrich Stahl (ed), Camille G. Wermuth (ed), ISBN:3-90639-026-8, Hardcover, 388 pages, August 2002. Generally, such salts can be prepared by reacting the free acid or base forms of these compounds with the appropriate base or acid in water or in an organic solvent, or in a mixture of the two; generally, non-aqueous media such as ether, ethyl acetate, ethanol, isopropanol, or acetonitrile are used.

Aspects of the invention also comprise therapeutic combination compositions, which can refer to a composition comprising a mixture of at least two different active compounds. For example, the active compound can be an elovanoid, a PAF-receptor antagonist, an anti-VEGF antibody, Suramab, or any combination thereof.

Therapeutic combination compositions can also comprise one or more additional agents, including but not limited to an excipient, diluent, or carrer as described herein. For example, the therapeutic combination can comprise a pharmaceutical composition comprising, for example, a first active compound (or anti-cancer agent), a second active compound (or anti-cancer agent), and a pharmaceutically acceptable carrier, excipient, or diluent.

Non-limiting examples of therapeutic combination compositions comprise:
a. a therapeutically effective amount of an elovanoid and a therapeutically effective amount of a PAF-receptor antagonist;
b. a therapeutically effective amount of an elovanoid and a therapeutically effective amount of an anti-VEGF antibody;
c. therapeutically effective amount of an elovanoid and a therapeutically effective amount of Suramab;
d. therapeutically effective amount of an elovanoid and a therapeutically effective amount of Suramin;
e. a therapeutically effective amount of a PAF-receptor antagonist and a therapeutically effective amount of an anti-VEGF antibody;
f. a therapeutically effective amount of a PAF-receptor antagonist and a therapeutically effective amount of Suramab;
g. a therapeutically effective amount of a PAF-receptor antagonist and a therapeutically effective amount of Suramin; or
h. any combination of the compositions described herein.

In embodiments, the disclosure provides formulations of pharmaceutical compositions containing therapeutically effective amounts of one or more of anti-cancer compounds provided herein or their salts thereof in a pharmaceutically acceptable carrier.

The provided compositions contain one or more compounds provided herein or their salts thereof, and a pharmaceutically acceptable excipient, diluent, carrier and/or adjuvant. The compounds can be formulated into suitable pharmaceutical preparations such as solutions, suspensions, tablets, dispersible tablets, pills, capsules, powders, sustained release formulations or elixirs, for oral, buccal, intranasal, vaginal, rectal, ocular administration, sustained release from intravitreal implanted reservoirs or nano-devices or dendrimers, embedded in collagen or other materials on a tissue surface, or in sterile solutions or suspensions for parenteral administration, dermal patches as well as transdermal patch preparation and dry powder inhalers. The compounds described herein can be formulated into pharmaceutical compositions using techniques and procedures well known in the art (see, e.g., Ansel Introduction to Pharmaceutical Dosage Forms, Fourth Edition 1985, 126).

Embodiments of the disclosure provide pharmaceutical compositions containing various forms of the provided compounds, such as pharmaceutically acceptable salts or derivatives thereof.

In embodiments, the disclosure provides a pharmaceutical composition for the treatment of cancer. In embodiments, the cancer is a brain cancer, such as glioblastoma multiforme.

In embodiments, effective concentrations of one or more anti-cancer compounds or pharmaceutically acceptable derivatives is (are) mixed with a suitable pharmaceutical carrier or vehicle. The compounds can be derivatized as the corresponding salts, esters, enol ethers or esters, acids, bases, solvates, hydrates or prodrugs prior to formulation, as described above. The concentrations of the compounds in the compositions can be effective for delivery of an amount, upon administration, that treats, prevents, or ameliorates one or more of the symptoms of a disease, disorder or condition, such as cancer.

As described herein, the compositions can be readily prepared by adapting methods known in the art. The compositions can be a component of a pharmaceutical formulation. The pharmaceutical formulation can further contain known agents for the treatment of diseases such as cancer, or symptoms thereof.

Embodiments also provides packaged composition(s) or pharmaceutical composition(s) for prevention, restoration, or use in treating the disease or condition. Other packaged compositions or pharmaceutical compositions can further include indicia including at least one of: instructions for using the composition to treat the disease or condition. The kit can further include appropriate buffers and reagents known in the art for administering various combinations of the components listed above to the host.

Embodiments herein include a composition or pharmaceutical composition that can be formulated with one or more pharmaceutically acceptable excipients, diluents, carriers, naturally occurring or synthetic antioxidants, and/or adjuvants. In addition, embodiments can include a composition or pharmaceutical composition formulated with one or more pharmaceutically acceptable auxiliary substances. In particular the composition or pharmaceutical composition can be formulated with one or more pharmaceutically acceptable excipients, diluents, carriers, and/or adjuvants to provide an embodiment of a composition of the disclosure.

A wide variety of pharmaceutically acceptable excipients are known in the art. Pharmaceutically acceptable excipients have been amply described in a variety of publications, including, for example, A. Gennaro (2000) “Remington: The Science and Practice of Pharmacy,” 20th edition, Lippincott, Williams, & Wilkins; Pharmaceutical Dosage Forms and Drug Delivery Systems (1999) H.C. Ansel et al., eds., 7th ed., Lippincott, Williams, & Wilkins; and Handbook of Pharmaceutical Excipients (2000) A.H. Kibbe et al., eds., 3rd ed. Amer. Pharmaceutical Assoc. The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

In embodiments, the composition or pharmaceutical composition can be administered to the subject using any means capable of resulting in the desired effect, for example, preventing or treating cancer. Thus, the composition or pharmaceutical composition can be incorporated into a variety of formulations for therapeutic administration. For example, the composition or pharmaceutical composition can be formulated into pharmaceutical compositions by combination with appropriate, pharmaceutically acceptable carriers or diluents, and can be formulated into preparations in solid, semi-solid, liquid or gaseous forms, such as tablets, capsules, powders, granules, ointments, solutions, suppositories, injections, inhalants and aerosols.

Suitable excipient vehicles for the composition or pharmaceutical composition are, for example, water, saline, dextrose, glycerol, ethanol, or the like, and combinations thereof. In addition, if desired, the vehicle can contain minor amounts of auxiliary substances such as wetting or emulsifying agents, antioxidants or pH buffering agents. Methods of preparing such dosage forms are known, or will be apparent upon consideration of this disclosure, to those skilled in the art. See, e.g., Remington’s Pharmaceutical Sciences, Mack Publishing Company, Easton, Pennsylvania, 17th edition, 1985. The composition or formulation to be administered will, in any event, contain a quantity of the composition or pharmaceutical composition adequate to achieve the desired state in the subject being treated.

Compositions can include those that comprise a sustained release or controlled release matrix. In addition, embodiments can be used in conjunction with other treatments that use sustained-release formulations. As used herein, a sustained-release matrix is a matrix made of materials, usually polymers, which are degradable by enzymatic or acid-based hydrolysis or by dissolution. Once inserted into the body, the matrix is acted upon by enzymes and body fluids. A sustained-release matrix desirably is chosen from biocompatible materials such as liposomes, polylactides (polylactic acid), polyglycolide (polymer of glycolic acid), polylactide co-glycolide (copolymers of lactic acid and glycolic acid), polyanhydrides, poly(ortho)esters, polypeptides, hyaluronic acid, collagen, chondroitin sulfate, carboxylic acids, fatty acids, phospholipids, polysaccharides, nucleic acids, polyamino acids, amino acids such as phenylalanine, tyrosine, isoleucine, polynucleotides, polyvinyl propylene, polyvinylpyrrolidone and silicone. Illustrative biodegradable matrices include a polylactide matrix, a polyglycolide matrix, and a polylactide co-glycolide (co-polymers of lactic acid and glycolic acid) matrix. In another embodiment, the pharmaceutical composition (as well as combination compositions) can be delivered in a controlled release system. For example, the composition or pharmaceutical composition can be administered using intravenous infusion, an implantable osmotic pump, a transdermal patch, liposomes, or other modes of administration. In one embodiment, a pump may be used (Sefton (1987). CRC Crit. Ref. Biomed. Eng. 14:201; Buchwald et al. (1980). Surgery 88:507; Saudek et al. (1989). N. Engl. J. Med. 321:574). In another embodiment, polymeric materials are used. In yet another embodiment a controlled release system is placed in proximity of the therapeutic target thus requiring only a fraction of the systemic dose. In yet another embodiment, a controlled release system is placed in proximity of the therapeutic target, thus requiring only a fraction of the systemic. Other controlled release systems are discussed in the review by Langer (1990). Science 249:1527-1533.

In another embodiment, the compositions of the present disclosure (as well as combination compositions separately or together) include those formed by impregnation of the composition or pharmaceutical composition described herein into absorptive materials, such as sutures, bandages, and gauze, or coated onto the surface of solid phase materials, such as surgical staples, zippers and catheters to deliver the compositions. Other delivery systems of this type will be readily apparent to those skilled in the art in view of the instant disclosure.

In another embodiment, the compositions or pharmaceutical compositions (as well as combination compositions separately or together) can be part of a delayed-release formulation. Delayed-release dosage formulations can be prepared as described in standard references such as “Pharmaceutical dosage form tablets”, eds. Liberman et. al. (New York, Marcel Dekker, Inc., 1989), “Remington-The science and practice of pharmacy”, 20th ed., Lippincott Williams & Wilkins, Baltimore, MD, 2000, and “Pharmaceutical dosage forms and drug delivery systems”, 6th Edition, Ansel et al., (Media, PA: Williams and Wilkins, 1995). These references provide information on excipients, materials, equipment and process for preparing tablets and capsules and delayed release dosage forms of tablets, capsules, and granules. These references provide information on carriers, materials, equipment and process for preparing tablets and capsules and delayed release dosage forms of tablets, capsules, and granules.

### Methods of Treatment

Aspects of the invention are directed towards methods of treating or preventing cancer.

As used herein, “treatment” and “treating” can refer to the management and care of a subject for the purpose of combating a condition, disease, or disorder, in any manner in which one or more of the symptoms of a disease or disorder are ameliorated or otherwise beneficially altered. The term can include the full spectrum of treatments for a given condition from which the patient is suffering, such as administration of the active compound for the purpose of: alleviating or relieving symptoms or complications; delaying the progression of the condition, disease or disorder; curing or eliminating the condition, disease or disorder; and/or preventing the condition, disease or disorder, wherein “preventing” or “prevention” can refer to the management and care of a patient for the purpose of hindering the development of the condition, disease or disorder, and includes the administration of the active compounds to prevent or reduce the risk of the onset of symptoms or complications. The patient to be treated can be a mammal, such as a human being. Treatment also encompasses any pharmaceutical use of the compositions herein, such as use for treating a disease as provided herein.

The term “treatment of cancer” or “treating cancer” can refer to the prevention or alleviation or amelioration of any of the phenomena known in the art to be associated with the pathology commonly known as “cancer.” The term “cancer” can refer to the spectrum of pathological symptoms associated with the initiation or progression, as well as metastasis, of malignant tumors. The term “tumor” can refer to a new growth of tissue in which the multiplication of cells is uncontrolled and progressive. In embodiments, the tumor can be a malignant tumor, one in which the primary tumor has the properties of invasion or metastasis or which shows a greater degree of anaplasia than do benign tumors. Thus, “treatment of cancer” or “treating cancer” can refer to an activity that prevents, alleviates or ameliorates any of the primary phenomena (initiation, progression, metastasis) or secondary symptoms associated with the disease.

Treating cancer can be indicated by, for example, inhibiting or delaying invasiveness of a cancer. “Cancer invasion” can refer to the movement caused by cancer cells in vivo, into or through biological tissue or the like. For example, movements caused by cancer cells into or through barriers formed by special cell-based proteins, such as collagen and Matrigel, and other substances.

The phrase “preventing cancer” can refer to prevention of cancer occurrence. In embodiments, the preventative treatment reduces the recurrence of the cancer. In other embodiments, preventative treatment decreases the risk of a patient from developing a cancer, or inhibits progression of a pre-cancerous state (e.g. acolon polyp) to actual malignancy.

Embodiments as described herein can prevent, inhibit, or delay angiogenesis. As such, compounds and compositions described herein can be referred to as “anti-angiogenic”. Angiogenesis can refer to the migration and formation of capillaries by endothelial cells. Growth can refer to an increase in cell size, shape, and complexity. Proliferation can refer to the growth of cells by cell division. Differentiation can refer to the process by which cells change from an undifferentiated state to a more differentiated state, usually in association with diverse functional roles and the development of new and diverse traits. Cellular interactions can refer to alternations of cellular behavior such as migration, invasion, angiogenesis, growth, proliferation or differentiation in response to presence and movement between nearby homologous or heterologous cells.

“Cancer” can refer to diseases in which abnormal cells divide without control and can invade nearby tissues. Cancer cells can also spread to other parts of the body through the blood and lymph systems. There are several main types of cancer. Carcinoma is a cancer that begins in the skin or in tissues that line or cover internal organs. Sarcoma is a cancer that begins in bone, cartilage, fat, muscle, blood vessels, or other connective or supportive tissue. Leukemia is a cancer that starts in blood-forming tissue, such as the bone marrow, and causes large numbers of abnormal blood cells to be produced and enter the blood. Lymphoma and multiple myeloma are cancers that begin in the cells of the immune system. Central nervous system cancers are cancers that begin in the tissues of the brain and spinal cord. Also called malignancy.

In embodiments, the cancer can comprise a solid tumor or a liquid cancer. A “solid tumor” can refer to an abnormal mass of tissue that usually does not contain cysts or liquid. A “non-solid tumor”, which can be referred to as a “liquid cancer”, can refer to neoplasia of the hemopoietix system, such as lymphoma, myeloma, and leukemia, or neoplasia without solid formation generally and with spread substantially.

In some embodiment, the solid tumors include but not limited to brain cancer, lung cancer, liver cancer, hepatocellular carcinoma (HCC), esophageal cancer, cholangiocarcinoma, gallbladder carcinoma, stomach cancer, abdominal cancer, gastrointestinal cancer, gastric cancer, pancreatic cancer, renal cell carcinoma, renal cancer, bone cancer, breast cancer, ovarian cancer, uterine cancer, cervical cancer, endometrial cancer, colorectal cancer, colon cancer, rectal cancer, bladder cancer, superficial bladder cancer, prostate cancer, adrenal tumors, squamous cell carcinoma, neuroma, malignant neuroma, myoepithelial carcinoma, synovial sarcoma, rhabdomyosarcoma, gastrointestinal interstitial cell tumor, skin cancer, basal cell carcinoma, malignant melanoma, thyroid cancer, nasopharyngeal carcinoma, hemangioma, epidermoid carcinoma, head and neck cancer, glioma, or Kaposi’s sarcoma.

In some embodiments, the non-solid tumors include but not limited to leukemia, acute leukemia, chronic leukemia, chronic myelocytic leukemia, acute myelogenous leukemia, chronic myelogenous leukemia, chronic lymphocytic leukemia, acute lymphoblastic leukemia, T-cell leukemia, hairy cell leukemia, polycythemia, myelodysplastic syndrome, multiple myeloma, lymphadenoma, Hodgkin’s lymphoma, and Non-Hodgkin’s lymphoma.

In some embodiments, the tumors are brain tumors, and include but not limited to abnormally proliferative or aberrantly proliferative brain cells and/or malignant brain tumor. As used herein, the term “brain tumor” can refer to any brain tumors, therefore, such tumors can be primary brain tumors and/or metastatic brain tumors, for example, the tumors are formed in a way that tumors at other positions are metastasized to the brain through various metastasis modes. The brain tumors can be benign (non-carcinous), preinvasive lesion (precancerous lesion), or malignant (carcinous) brain tumors, such as brain cancers.

The term “brain tumor” can refer to a glioma or primary brain tumor derived from glial support cells, and which is the most common primary tumor of the adult central nervous system resulting in an estimated 13,000 deaths in 2010. Adult gliomas of astrocytic origin (astrocytomas) comprise a spectrum of neoplasms that are generally classified by WHO standards into low-grade benign tumors (i.e., juvenile pilocytic astrocytoma, diffuse astrocytoma) and high-grade malignant tumors (i.e., anaplastic astrocytoma and glioblastoma multiforme (GBM)). Patients diagnosed with grade IV GBM, the most aggressive malignant glioma, have a median survival of 9-12 months after the onset of clinical symptoms. Molecular analyses of glioma specimens have identified several common genetic alterations that may contribute to glioblastoma formation.

The term “Glioblastoma Multiforme (GBM)” as used herein is a glioma or brain tumor derived from glial cells characterized by the presence of small areas of necrotizing tissue that is surrounded by anaplastic cells (pseudopalisading necrosis). This characteristic, as well as the presence of hyperplastic blood vessels, differentiates the tumor from Grade 3 astrocytomas, which do not have these features. In general, gliomas are extremely difficult to treat using conventional approaches. This is primarily due to the intrinsic propensity of glioma cells to exit the tumor core and invade the adjacent normal brain parenchyma. These migrating cells escape surgical resection and are poorly targeted by radiation or chemotherapy. They sometimes travel over long distances, frequently along blood vessel and fiber tracts, and then initiate secondary tumor growth at their final destination. This distinguishing invasive ability is not shared by non-glial cells that metastasize from other primary tumor sites (e.g., breast) to brain tissue. The invasion of glioma cells is likely triggered by a presently undefined signal or signals that promote a cascade of cellular responses, including cell elongation, integrinmediated cell attachment to extracellular matrix (ECM) molecules, the production and secretion of ECM - degrading enzymes, and cell movement.

In some embodiments of this aspect of the disclosure, the brain tumor can be selected from the group consisting of: a glioblastoma, an astrocytoma, an oligodendroglioma, an ependymal tumor, a neuronal tumor and a combination of glial tumors.

In embodiments, the method can comprise administering to a subject a therapeutically effective amount of a composition described herein.

The term “administration” can refer to introducing a pharmaceutical composition or formulation as described herein into a subject. One route of administration of the composition is intravenous administration. However, any route of administration, such as oral, topical, subcutaneous, peritoneal, intra-arterial, inhalation, vaginal, rectal, nasal, introduction into the cerebrospinal fluid, or instillation into body compartments can be used.

The formulation or pharmaceutical compound can be administered alone, but can be administered with other compounds, excipients, fillers, binders, carriers or other vehicles selected based upon the chosen route of administration and standard pharmaceutical practice. Administration may be by way of carriers or vehicles, such as injectable solutions, including sterile aqueous or non-aqueous solutions, or saline solutions; creams; lotions; capsules; tablets; granules; pellets; powders; suspensions, emulsions, or microemulsions; patches; micelles; liposomes; vesicles; implants, including microimplants; eye drops; other proteins and peptides; synthetic polymers; microspheres; nanoparticles; and the like.

The formulations or pharmaceutical composition can also be included, or packaged, with other non-toxic compounds, such as pharmaceutically acceptable carriers, excipients, binders and fillers including, but not limited to, glucose, lactose, gum acacia, gelatin, mannitol, xanthan gum, locust bean gum, galactose, oligosaccharides and/or polysaccharides, starch paste, magnesium trisilicate, talc, corn starch, starch fragments, keratin, colloidal silica, potato starch, urea, dextrans, dextrins, and the like. For example, the pharmaceutically acceptable carriers, excipients, binders, and fillers that can be used include those which render the compounds of the invention amenable to intravitreal delivery, intraocular delivery, ocular delivery, subretinal delivery, intrathecal delivery, intravenous delivery, subcutaneous delivery, transcutaneous delivery, intracutaneous delivery, intracranial delivery, topical delivery and the like. Moreover, the packaging material can be biologically inert or lack bioactivity, such as plastic polymers, and silicone, and can be processed internally by the subject without affecting the effectiveness of the composition/formulation packaged and/or delivered therewith.

Different forms formulation can be calibrated in order to adapt both to different individuals and to the different needs of a single individual. Implementing this concept is complicated, and the necessary research is challenging. However, the present formulation need not counter every cause in every individual. Rather, by countering the necessary causes, the present formulation will restore the body and brain to their normal function. Then the body and brain themselves will correct the remaining deficiencies. No drug can possibly correct every single aspect of cancer, but the present formulation will maximize the possibility.

“Parenteral administration” can refer to administration via injection or infusion. Parenteral administration includes, but is not limited to, subcutaneous administration, intravenous administration, intramuscular administration.

For oral preparations, the composition or pharmaceutical composition can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

Embodiments of the composition or pharmaceutical composition can be formulated into preparations for injection by dissolving, suspending, or emulsifying them in an aqueous or non-aqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

Embodiments of the composition or pharmaceutical composition can be utilized in aerosol formulation to be administered via inhalation. Embodiments of the composition or pharmaceutical composition can be formulated into pressurized acceptable propellants such as dichiorodifluoromethane, propane, nitrogen and the like.

Unit dosage forms for oral administration, such as syrups, elixirs, and suspensions, can be provided wherein each dosage unit, for example, teaspoonful, tablespoonful, tablet or suppository, contains a predetermined amount of the composition containing one or more compositions. Similarly, unit dosage forms for injection or intravenous administration may comprise the composition or pharmaceutical composition in a composition as a solution in sterile water, normal saline or another pharmaceutically acceptable carrier.

Embodiments of the composition or pharmaceutical composition can be formulated in an injectable composition in accordance with the disclosure. For example, injectable compositions are prepared as liquid solutions or suspensions; solid forms suitable for solution in, or suspension in, liquid vehicles prior to injection can also be prepared. The preparation can also be emulsified or the active ingredient (triamino-pyridine derivative and/or the labeled triamino-pyridine derivative) encapsulated in liposome vehicles in accordance with the present disclosure.

In an embodiment, the composition or pharmaceutical composition can be formulated for delivery by a continuous delivery system. The term “continuous delivery system” is used interchangeably herein with “controlled delivery system” and encompasses continuous (e.g., controlled) delivery devices (e.g., pumps) in combination with catheters, injection devices, and the like, a wide variety of which are known in the art.

Embodiments of the composition or pharmaceutical composition can be administered to a subject in one or more doses. Those of skill will readily appreciate that dose levels can vary as a function of the specific composition or pharmaceutical composition administered, the severity of the symptoms and the susceptibility of the subject to side effects. Dosages for a given compound are readily determinable by those of skill in the art by a variety of means.

In an embodiment, multiple doses of the composition or pharmaceutical composition are administered. The frequency of administration of the composition or pharmaceutical composition can vary depending on any of a variety of factors, e.g., severity of the symptoms, and the like. For example, in an embodiment, the composition or pharmaceutical composition can be administered once per month, twice per month, three times per month, every other week (qow), once per week (qw), twice per week (biw), three times per week (tiw), four times per week, five times per week, six times per week, every other day (qod), daily (ad), twice a day (qid), three times a day (tid), or four times a day. As discussed above, in an embodiment, the composition or pharmaceutical composition is administered 1 to 4 times a day over a 1 to 10-day time period.

The duration of administration of the composition or pharmaceutical composition analogue, e.g., the period of time over which the composition or pharmaceutical composition is administered, can vary, depending on any of a variety of factors, including patient response. For example, the composition or pharmaceutical composition in combination or separately, can be administered over a period of time of about one day to one week, about one day to two weeks. The amount of the PAF antagonist and pharmaceutical compositions of the disclosure that can be effective in treating the condition or disease can be determined by standard clinical techniques. In addition, in vitro or in vivo assays can optionally be employed to help identify optimal dosage ranges. The precise dose to be employed can also depend on the route of administration, and can be decided according to the judgment of the practitioner and each patient’s circumstances.

In embodiments, two or more anti-cancer agents can be administered sequentially, such as one before the other, or concurrently or simultaneously, such as at about the same time. The term “simultaneous administration”, as used herein, indicates that the first anti-cancer agent and the second anti-cancer agent in the therapeutic combination therapy are administered either less than about 15 minutes, e.g., less than about 10, 5, or 1 minute. When the first and second anti-cancer agents are administered simultaneously, the first and second treatments can be in the same composition (e.g., a composition comprising both the first and second therapeutic agents) or separately (e.g., the first therapeutic agent is contained in one composition and the second treatment is contained in another composition).

As used herein, the term “sequential administration” can indicate that the first anti-cancer agent and the second anti-cancer agent in combination therapy are greater than about 15 minutes, such as greater than about 20, 30, 40, 50, 60 minutes, or greater than 60 minutes. Either the first anti-cancer agent or the second anti-cancer agent can be administered first. The first and second anti-cancer agents are included in separate compositions, which can be included in the same or different packages or kits.

As used herein, the term “simultaneous administration” means that administration of a first therapeutic agent and a second therapeutic agent in a combination therapy overlap each other.

In embodiments, non-limiting examples of administration of treatment can comprise administering a PAF-r antagonist first followed by a Suramab, an anti-VEGF antibody, an elovanoid, or any combination thereof. For example, the PAF-r antagonist can be LAU-0901, LAU-09015, LAU-09017, LAU-09017, LA-09018, LAU-09019, LAU-09020, LAU-09021, LAU-09023, or LAU-09025. For example, the anti-VEGF antibody can be bevacizumab, ramucirumab, or ranibizumab.

For example, the elovanoid can be ELV 32:6 or ELV 34:6. For example, LAU-0901 can be administered first, followed by bevacizumb, suramab, ELV, or a combination there of about 5 minutes afterward. For example, ELV can be administered followed by bevacizumb or Suramab or LAU-0901 about 5 minutes later.

The therapeutic compositions of the disclosure provide methods and compositions for the administration of the active agent(s) to a subject using any available method and route suitable for drug delivery, including in vivo, in vitro and ex vivo methods, as well as systemic and localized routes of administration. Routes of administration include intranasal, intramuscular, intratracheal, subcutaneous, intra cerebroventricular, intradermal, topical application, intravenous, rectal, nasal, oral, and other enteral and parenteral routes of administration. Routes of administration can be combined, if desired, or adjusted depending upon the agent and/or the desired effect. An active agent can be administered in a single dose or in multiple doses.

Embodiments of the composition or pharmaceutical composition can be administered to a subject using available conventional methods and routes suitable for delivery of conventional drugs, including systemic or localized routes. Routes of administration can include, but are not limited to, enteral administration, parenteral administration, or inhalation.

Other compositions, compounds, methods, features, and advantages of the disclosure will be or become apparent to one having ordinary skill in the art upon examination of the following drawings, detailed description, and examples. It is intended that all such additional compositions, compounds, methods, features, and advantages be included within this description, and be within the scope of the disclosure.

Anti-cancer agents and compositions as described herein can be administered locally or systemically. “Local administration” can refer to administering a composition or drug into a limited or partial anatomy space. Examples of local administration include but are not limited to intraturmoral, intra-lymph node, intra-pleural space, intraperitoneal cavity and the like. “Systemic administration” can refer to administration of an anti-cancer agent such that the anti-cancer agent becomes widely distributed in the body in significant amounts and has a biological effect, e.g., its desired effect, in the blood and/or reaches its desired site of action via the vascular system. For example, systemic routes of administration include administration by (1) introducing the agent directly into the vascular system or (2) oral, pulmonary, or intramuscular administration wherein the agent is adsorbed, enters the vascular system, and is carried to one or more desired site(s) of action via the blood.

### Kits

Aspects of the invention are directed towards kits, such as kits comprising compositions as described herein for treating cancer. For example, the kit can comprise therapeutic combination compositions described herein.

In one embodiment, the kit includes (a) a container that contains an anti-cancer composition, such as that described herein, and optionally (b) informational material. The informational material can be descriptive, instructional, marketing or other material that relates to the methods described herein and/or the use of the agents for therapeutic benefit.

In an embodiment, the kit includes two or more anti-cancer agents. For example, the kit includes a first container that contains a composition that includes an elovanoid, and a second container that includes a PAF-antagonist.

The informational material of the kits is not limited in its form. In one embodiment, the informational material can include information about production of the compound, molecular weight of the compound, concentration, date of expiration, batch or production site information, and so forth. In one embodiment, the informational material relates to methods of administering the therapeutic combination composition, e.g., in a suitable dose, dosage form, or mode of administration (e.g., a dose, dosage form, or mode of administration described herein), to treat a subject who has a nerve disconnectivity disorder). The information can be provided in a variety of formats, include printed text, computer readable material, video recording, or audio recording, or information that provides a link or address to substantive material.

The composition in the kit can include other ingredients, such as a solvent or buffer, a stabilizer, or a preservative. The antagonist can be provided in any form, e.g., liquid, dried or lyophilized form, preferably substantially pure and/or sterile. When the agents are provided in a liquid solution, the liquid solution preferably is an aqueous solution. When the agents are provided as a dried form, reconstitution generally is by the addition of a suitable solvent. The solvent, e.g., sterile water or buffer, can optionally be provided in the kit.

The kit can include one or more containers for the composition or compositions containing the agents. In some embodiments, the kit contains separate containers, dividers or compartments for the composition and informational material. For example, the composition can be contained in a bottle, vial, or syringe, and the informational material can be contained in a plastic sleeve or packet. In other embodiments, the separate elements of the kit are contained within a single, undivided container. For example, the composition is contained in a bottle, vial or syringe that has attached thereto the informational material in the form of a label. In some embodiments, the kit includes a plurality (e.g., a pack) of individual containers, each containing one or more unit dosage forms (e.g., a dosage form described herein) of the agents. The containers can include a combination unit dosage, e.g., in a desired ratio. For example, the kit includes a plurality of syringes, ampules, foil packets, blister packs, or medical devices, e.g., each containing a single combination unit dose. The containers of the kits can be air tight, waterproof (e.g., impermeable to changes in moisture or evaporation), and/or light-tight. The kit optionally includes a device suitable for administration of the composition, e.g., a syringe or other suitable delivery device. The device can be provided pre-loaded with one or both of the agents or can be empty, but suitable for loading.

### EXAMPLES

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only, since alternative methods can be utilized to obtain similar results.

### Example 1

### Example 1 - Methods to Prevent Glioblastoma Multiform Brain Invasiveness

An aspect of the invention is the use of systemically injected Suramab (Suramin plus Avastin) or LAU-0901 plus Elovanoids or plus Suramab to block/downregulate GBM infiltration in the brain. Experimentally (in a mouse model), we show that these treatments improve survival, reduce brain tumor size, and reduce mortality.

Mechanistically our new approach targets several pathways and neuroinflammatory disruptions triggered by brain tumor. The small molecule LAU-0901 suppresses pro-inflammatory signaling by antagonizing the platelet-activating factor receptor (PAFR). Elovanoids are a new class of endogenous pro-homeostatic lipid mediators, which exhibit neuroprotective bioactivities and inactivate pro-apoptotic and pro-inflammatory signaling in experimental stroke, neurons in culture and retinal cells needed to sustain photoreceptor integrity. Suramin has antitumor activity, inhibiting the binding of multiple growth factors, and inhibiting glutamatergic synaptic transmission.

Glioblastoma Multiforme (GBM) is the most aggressive primary brain tumor with a poor prognosis and average survival of 13-16 months with the current standard of treatment: surgical resection, radiation, and chemotherapy. The inefficacy of current treatments necessitates the development of novel GBM therapeutics that effectively blocks infiltration/invasiveness. Our data demonstrate the experimental therapeutic usefulness of our compounds when applied to a GBM model.

The compounds selected are a new and different approach to block GBM invasiveness/infiltration. Their action is effective and timely resolution of inflammation and activation of neuroprotective pathways, including blunting proangiogenic events. Without wishing to be bound by theory, they will offer an effective treatment option to reduce GBM invasiveness.

### Example 2

### Example 2 - New Experimental Therapies for Glioblastoma Multiforme

Glioblastoma multiforme (GBM) is the most common and aggressive primary malignant brain tumor in adults, and despite surgical resection, chemotherapy, and irradiation the average survival rate is 13-16 months (Mooney et al., 2019). GBM is a high grade glioma originating from the glial cells of the central nervous system, accounting for 80.7% of all malignant brain tumors (Chen et al., 2019). The current standard of care which includes maximal surgical resection and adjuvant chemoradiation has shown little efficacy on patient survival, with median survival of less than 6 months following recurrence (Mooney et al., 2019). The only approved medication for the treatment of newly diagnosed GBM is temozolomide (TMZ) (Mukherji, 2010), and for recurrent GBM, bevacizumab or Avastin (Mukherji, 2010).

Development of effective therapeutics for GBM presents a challenge due to limitations for radiation therapy and chemotherapy arising from GBM cells multidrug resistance and small dose requirements for cytotoxic therapeutics (Han et al., 2015; Florman et al., 2016). Due to the complexity of the tumor structure as well as its infiltrative and rapidly proliferating nature the efficacy of surgical resection and radiation therapy is limited, ultimately leading to recurrence (Soda et al., 2013; Marrero et al., 2014; von Neubeck et al., 2015). Within the tumor, the vasculature is disorganized and highly permeable compromising the blood brain barrier (BBB), resulting in edema which causes patients to exhibit severe symptoms. However, in contrast to the tumor center, the BBB on the edges of the tumor remain uncompromised and functional, making drug delivery to these areas especially challenging (Soda et al., 2013). Another challenge therapeutic development faces is the acquired resistance to therapies GBM has been shown to exhibit. Current therapies aimed to inhibit tumor angiogenesis have been shown to lead to an adaptive tumor response, the most severe adaptation documented being the transition to a more invasive phenotype (Bergers and Hanahan, 2008).

Given the inefficacy of the current available therapeutics and the high incidence of recurrence, the necessity for the development of successful new therapeutics is critical. Two new treatments (LAU-0901, Elovanoid 34:6 and their combination) and Suramab (Suramin + Avastin) (Dumas and Bisser, 1999; Bouteille et al., 2003; Kappagoda et al., 2011; Lopex et al, 2011; Quinteros et al; 2016; Lopex et al, 2018) were investigated as a treatments for GBM. Our treatments were compared to Avastin, the current approved medication for treatment of GBM patients.

### LAU-0901

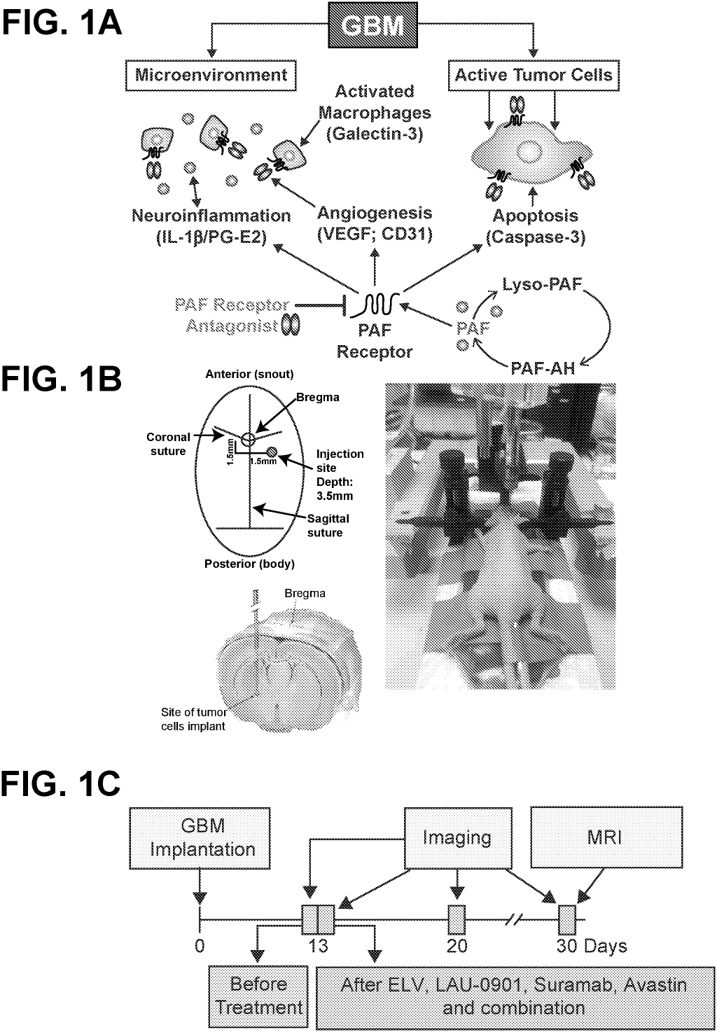

LAU-0901 (2,4,6-trimethyl-1, 4-dihydro-pyridine-3, 5-dicarboxylic acid) is a highly selective platelet activating factor receptor (PAFR) antagonist, and a potent inhibitor of apoptosis and inflammatory response (Bazan, 2003; He and Bazan, 2006). LAU-0901 is highly protective when used as an anti-inflammatory in a variety of inflammation models (Esquenazi et al., 2004, 2009; He and Bazan, 2006). It has also been shown to exhibit neuroprotective bioactivity when applied to a model of ischemia-reperfusion injury in rats and mice (Esquenazi et al., 2009; Belayev et al., 2012). In previous studies it was observed to improve behavioral deficits and reduce total infarct volumes by 76% after 2 h of MCAo in rats, and increase intra-cerebral blood flow (ICBF) by 77% at 6 h after 1 h of MCAo in mice (Belayev et al., 2008). Premise of using PAF-R antagonists in GBM presented in FIG. 1A.

### Elovanoids

Elovanoids are a class of endogenous pro-homeostatic lipid mediators that have been observed to protect against excitotoxicity (Bhattacharjee et al., 2017; Bazan, 2018). Elovanoids are stereoselective mediators which can be made on demand and can be derived from very long-chain polyunsaturated fatty acids (VLC-PUFAₛ) (Calandria et al., 2015). It has been demonstrated that Elovanoids can exhibit neuroprotective bioactivities in both in vitro neuronal models and in vivo experimental ischemic stroke (Belayev et al., 2017). They have also been shown to have ability to inactivate pro-apoptotic and pro-inflammatory signaling in experimental stroke and neurodegenerative diseases (Harvey et al., 2015). As an example, we validated the use of Elovanoid 34:6 (ELV) on our GBM mouse model.

### Suramab

Suramab is a combination of Suramin and Avastin (Lopez et al 2011, Quinteros et al 2016, Lopez et al, 2018. Suramin is used in the treatment of African sleeping sickness (African trypanosomiasis) and river blindness (onchocerciasis), infections both caused by parasites (Dumas and Bisser, 1999; Kappagoda et al., 2011). Suramin works by causing the parasites to lose energy, which causes their death. Suramin is a non-selective P2 purinergic antagonist (pEC₅₀ values are 4.5 (P2×₂), 5.4 (P2Xs), 4.3 (P2Y ₂), 4.0 (P2Y₄) and 4.8 (P2Y₁₁)). Displays diverse biological actions. It has demonstrated antitumor activity, inhibiting the binding of multiple growth factors, and additionally inhibiting glutamatergic synaptic transmission (Dumas and Bisser, 1999; Kappagoda et al., 2011).

### Avastin (Bevacizumab)

Avastin is an anti-vascular endothelial growth factor (VEGF) monoclonal antibody that has demonstrated a radiographic response rate of up to 40% as a single agent or in combination with chemotherapy for recurrent GBM. It was approved for use in GBM in 2008. However, it has limited efficacy likely due to adaptive mutations in GBM (Bergers and Hanahan, 2008), leading to no improvement in overall survival (OS) when compared to standard of care plus radiation in GBM patients (Mukherji, 2010; Ozdemir-Kaynak et al., 2018).

### Methods

### Animals

Studies were performed according to the National Institutes of Health guidelines and in accordance with nationally accepted principles in the care and use of experimental animals. The Institutional Animal Care and Use Committee (IACUC) at the Louisiana State University Health Sciences Center (LSUHSC), New Orleans, approved the animal protocols used for this study. Athymic nude female mice (Charles Rivers Laboratories and Envigo), 6 to 8 weeks of age, were used in all experiments. Water and food were available for ad libitum consumption. All efforts were made to minimize pain and suffering and to reduce the number of mice used in these experiments.

### GBM Mouse Model Using U87MG Cell Line

Athymic nude female mice (Charles Rivers Laboratories and Envigo), 6 to 8 weeks of age, were anesthetized with a ketamine/xylazine cocktail solution (100 mg/kg; 10 mg/kg). Then, mice were secured in a stereotactic head frame and a midline 1 cm incision over the scalp was made. Natural tears lubricant was applied to the eyes. For each mouse, 5 × 10⁶ U87MG cells in 5 µL serum free Dulbecco’s Modified Eagle Medium (DMEM) tagged with a luciferase receptor gene were implanted into the right hippocampus using a 10 µL Hamilton syringe at the following coordinates, in reference to the bregma: 1.5 mm lateral, 1.5 mm posterior and 3.5 mm in depth; the needle was lowered to 3.5 mm and pulled up by 1 mm, prior to the injection (Marrero et al., 2014) (FIG. 1B). Rectal and cranial temperatures will be maintained at 36-37° C. during surgical procedure. The incision was closed using black monofilament nylon 5.0 sterile sutures and the area was cleaned with betadine solution. The mice were placed in individual cages and observed daily for weight, temperature, and locomotor changes. Mice were perfused at the end of the 30-day survival, brains removed and ex vivo MRI imaging were conducted. Experimental design and treatments presented in FIG. 1C.

### GBM Cell Line

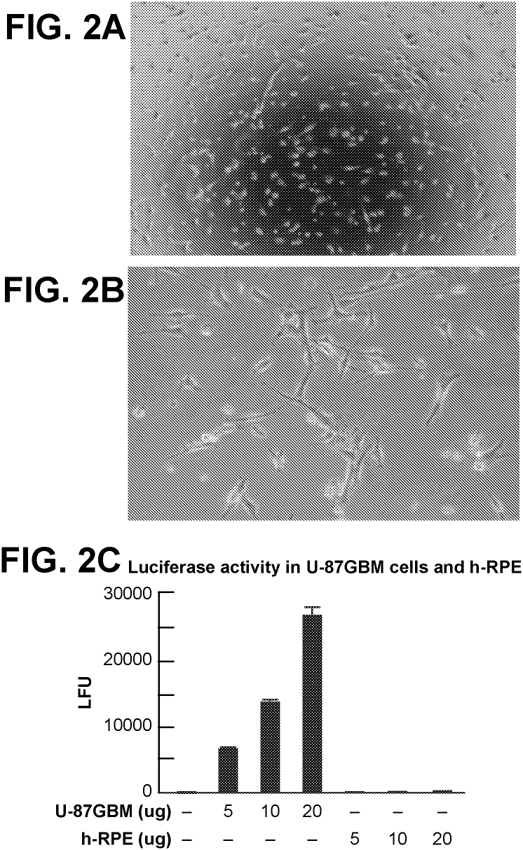

U87GBM cells were taken out from Cryofridze, and placed in a T-25 mM flask containing 5 ml DMEM-F12 (10%)-Glutamax medium and incubated at 37° C. Pictures were taken: 3 h, 36 h after incubation at 37° C. (FIGS. 2A and 2B, respectively). A luciferase receptor gene was used to tag the U87GBM cells. For detection of luciferase activity, approximately 500,000 U87GBM and human retinal pigment epithelial (h-RPE) cells were allowed to grow separately in six well plates for 36 hours (80% confluent) at 37° C. in triplicate. Cells were washed with cold phosphate-buffered saline (PBS) solution, harvested and cell extracts were made. Protein contents were measured by Bio-Rad method and adjusted. Luciferase activity was measured in 5-20 µg protein equivalent extracts using Luciferin as substrate. Luciferase units (LFU) was detected in Glomax 20/20 Luminometer. The luciferase assay showed that the activity is concentration dependent as luciferase activity gradually increased with the use of increasing concentration of cell extract from U87GBM cells. On the other hand, the h-RPE cell extracts did not show any luciferase activity under the similar experimental conditions (FIG. 2C). The luciferase assay results indicated that the GBM cell line used in our experiments are truly U87GBM cells as luciferase gene is tagged with this cell line. On the contrary, the h-RPE cell line was ineffective, which is expected since this cell line does not have the luciferase receptor gene.

### Treatments

Treatment was administered starting on day 13 after implantation, and intracranial tumor growth was quantified using in vivo bioluminescent imaging on days 13 (before treatment) and then 20 and 30. Mice were randomly allocated to experimental groups and treatments, which were performed in a blinded manner. Following treatments were used (n=5-7 per group): 1) LAU-0901 30 mg/kg, IP; daily x 5 days; 2) Elovanoid 34:6, 30 µg/mouse, IP; once; 3) Avastin 0.2 mg/mouse, IP, weekly x 2 doses; 4) Saline in equal volume (0.2 mL/mouse); 5) LAU-0901 + ELV; 6) LAU-0901 + Avastin; 7) LAU-0901 + Suramab, ELV 34:6 + Avastin; 8) Suramab (Suramin 10 mg/kg + Avastin 3 mg/kg); 9) ELV +Suramab. For combinatory treatment LAU-0901 was administered first, followed by Avastin, Suramab or ELV 5 min later. For ELV combinatory treatment: ELV first, followed by Avastin or Suramab 5 minutes later.

### In Vivo Bioluminescent Imaging

In vivo bioluminescent imaging was used to quantify and follow intracranial tumor growth at days 13, 20 and 30 post GBM implantation, using an in vivo imaging system provided by our Imaging Core Facility. This modality uses a Xenogen IVIS 200 system, which utilizes biphotonic imaging to non-invasively image, record, and quantitate cellular and genetic activity within an anesthetized organism. For each imaging session, mice were injected intraperitoneally with D-Luciferin (150 mg/kg) five minutes prior to imaging. Anesthesia was induced with isoflurane gas by placing mice in a chamber of an XGI-8 vaporizer, and sustained by inhalation via nose cones inside the imaging chamber. Images were captured and quantified with Living Image 4.1 software based on equivalent regions of interest over the head. Image intensities were expressed as photons per sec/cm² by procedures described previously by (Marrero et al., 2014). Growth imaging scores in Radiance (ROI) (p/sec/cm²/sr) were used to determine tumor growth and size.

### Ex Vivo MRI Data Collection and Analyses

30 days after completion of the experiment, mice had a perfusion with PBS, 4% PFA with added 8 mM Gd-DTPA (Gadobenate dimeglumine; 529 mg/ml; Henry Schein) per grams of body weight. Brains were removed and ex vivo MRI of mouse brains were conducted on a 11.7T Bruker Advance 8.9 cm horizontal bore instrument equipped with a 89 mm (ID) receiver coil (Bruker Biospin, Billerica, MA). Ex vivo MRI was obtained using a T1WI with the following parameters: TR/TE = 1000 ms/ 7 ms, matrix = 128², 25×0.5 mm slices, FOV=1.8 cm, NEX= 6. MR imaging acquisition time was ~9.5 min with an in-plane resolution of 234 um.

Cheshire image processing software (Hayden Image/Processing Group, Waltham, MA), was used to manually outline whole brain and tumor volumes enhanced by Gd deposition. T1WI data were optimized for signal intensity to enhance visualization of the tumors. Tumors were identified as hyperintense (T1WI) signal intensities within the striatum and surrounding tissues. Whole brain and tumor volumes (mm³) were extracted and analyzed (Jeffes et al., 2005; Blasiak et al., 2013).

### Statistical Analysis

Data are presented as mean values ± SEM. Repeated measures analysis of variance (ANOVA) followed by Bonferroni procedures to correct for multiple comparisons were used for intergroup comparisons. Two-tailed Student’s t tests were used for two-group comparisons. Differences at P<0.05 were considered statistically significant.

### Results

### Physiological Variables

No statistically significant differences in body weight or temperature was observed between groups. All animals in treatment groups survived with the exception of one vehicle animal.

### Effect of Elovanoid, Elovanoid+LAU-0901 and Elovanoid+Avastin Treatments

### In Vivo Bioluminescent Imaging Scores

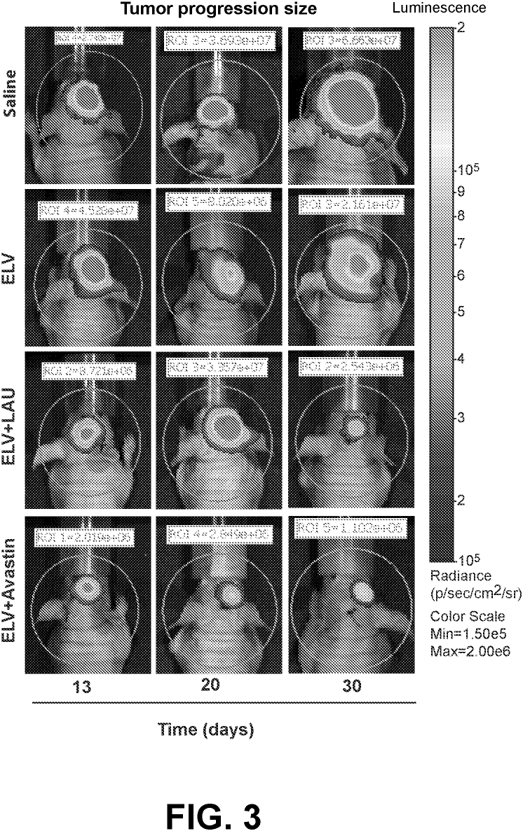

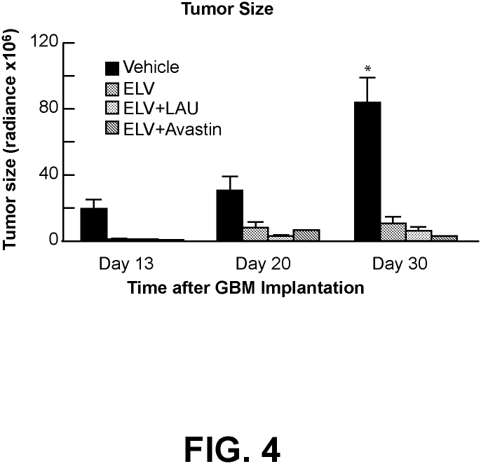

A significant reduction in growth and size was seen in Elovanoid, Elovanoid+LAU-0901 and Elovanoid+Avastin groups compared to the vehicle group on days 20 and 30 (FIGS. 3 and 4).

### Ex Vivo MRI Tumor Size

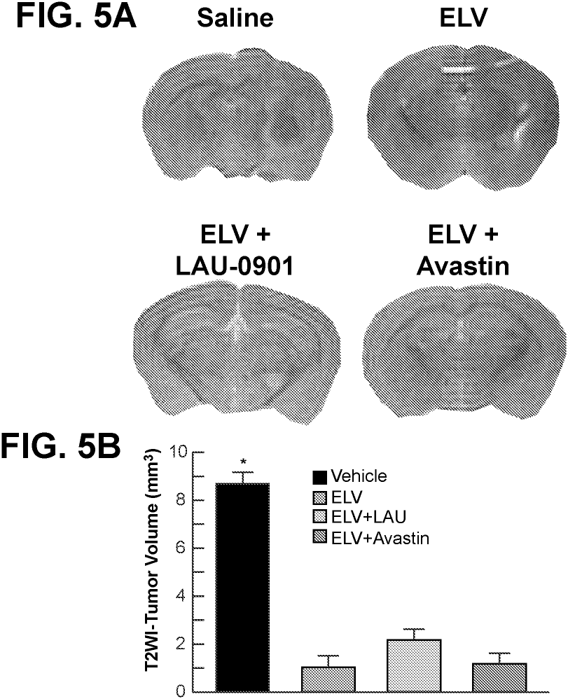

Tumor size was dramatically reduced by ELV and in combination with LAU-0901 and Avastin, resulted in reduced GBM tumor volumes compared to saline-treated mouse (FIGS. 5A and 5B).

### Effect of LA U-0901, Avastin and LA U-0901 + Avastin Treatments

### In Vivo Bioluminescent Imaging Scores

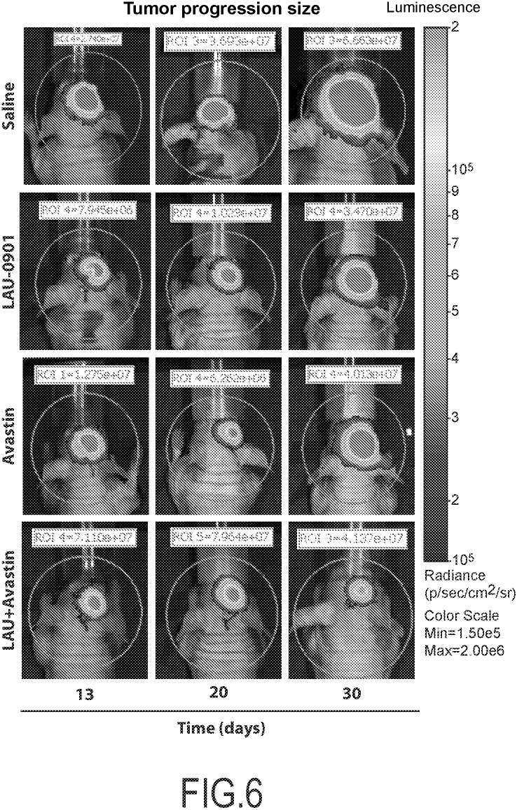

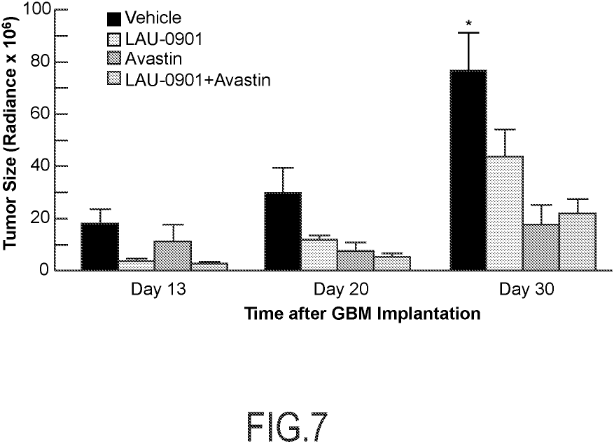

A significant reduction in growth and size was seen in LAU-0901, Avastin and LAU-0901+Avastin groups compared to the vehicle group on days 20 and 30 (FIGS. 6 and 7).

### Ex Vivo MRI Tumor Size

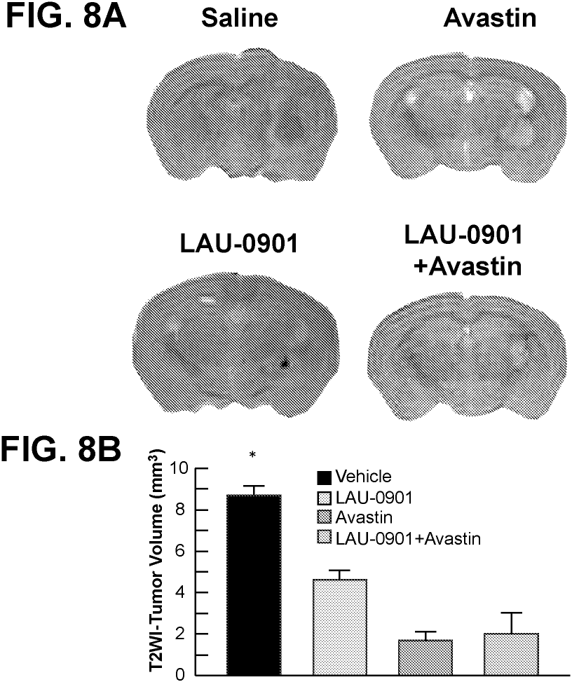

Tumor size was dramatically reduced by ELV and in combination with LAU-0901 and Avastin, resulted in reduced GBM tumor volumes compared to saline-treated mouse (FIGS. 8A and 8B).

### Effect of Suramab, Elovanoid +Suramab and LAU-0901 +Suramab Treatments.

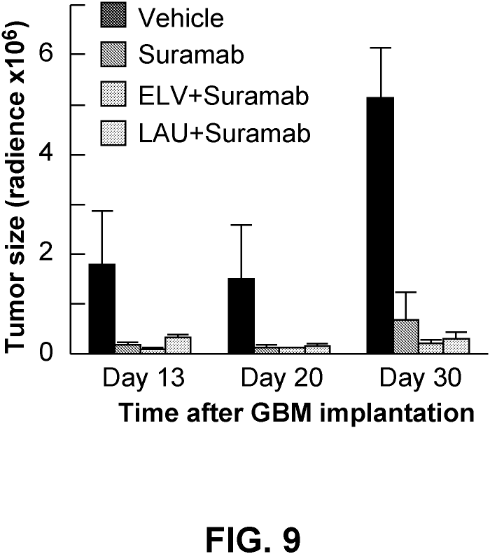

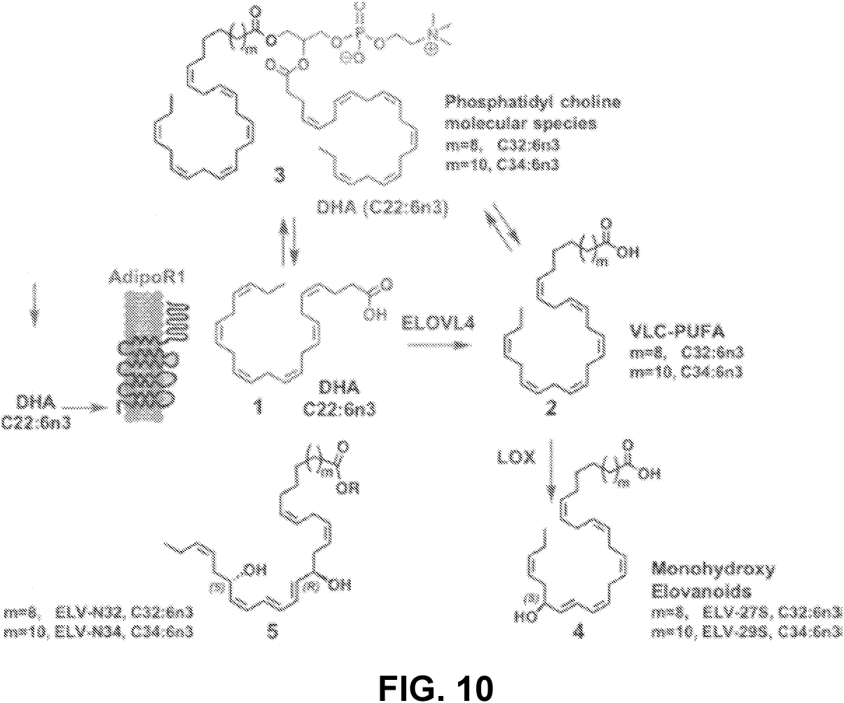

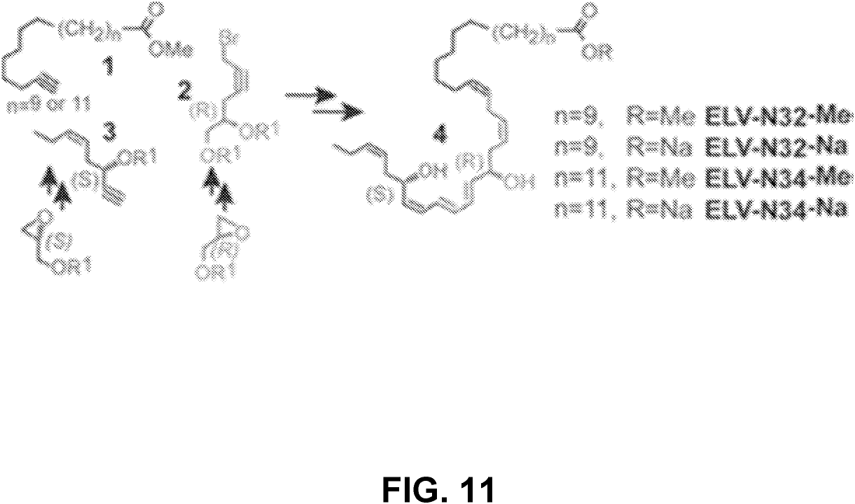

Tumor size was dramatically reduced by Suramab (94%) and ELV+ Suramab (98%), LAU-0901 + Suramab (97%) on day 30 compared to the vehicle (FIG. 9).

### Conclusion

Without wishing to be bound by theory, our results demonstrate the therapeutic use of these compounds and new compositions when applied to a GBM model.

### References Cited in This Example

Bazan, N. G. (2003). Synaptic lipid signaling: significance of polyunsaturated fatty acids and platelet-activating factor. J. Lipid Res. 44, 2221-2233. doi:10.1194/jlr.R300013-JLR200.

Bazan, N. G. (2018). Docosanoids and elovanoids from omega-3 fatty acids are pro-homeostatic modulators of inflammatory responses, cell damage and neuroprotection. Molecular Aspects of Medicine 64, 18-33. doi:10.1016/j.mam.2018.09.003.

Belayev, L., Eady, T. N., Khoutorova, L., Atkins, K. D., Obenaus, A., Cordoba, M., et al. (2012). Superior Neuroprotective Efficacy of LAU-0901, a Novel Platelet-Activating Factor Antagonist, in Experimental Stroke. TranslStroke Res 3, 154-163. doi:10.1007/s12975-011-0116-y.

Belayev, L., Khoutorova, L., Atkins, K., Gordon, W. C., Alvarez-Builla, J., and Bazan, N. G. (2008). LAU-0901, a novel platelet-activating factor antagonist, is highly neuroprotective in cerebral ischemia. Exp. Neurol. 214, 253-258. doi:10.1016/j.expneurol.2008.08.009.

Belayev, L., Mukherjee, P. K., Balaszczuk, V., Calandria, J. M., Obenaus, A., Khoutorova, L., et al. (2017). Neuroprotectin D1 upregulates Iduna expression and provides protection in cellular uncompensated oxidative stress and in experimental ischemic stroke. Cell Death Differ. 24, 1091-1099. doi:10.1038/cdd.2017.55.

Bergers, G., and Hanahan, D. (2008). Modes of resistance to anti-angiogenic therapy. Nature Reviews Cancer 8, 592-603. doi:10.108/nrc2442.

Bhattacharjee, S., Jun, B., Belayev, L., Heap, J., Kautzmann, M.-A., Obenaus, A., et al. (2017). Elovanoids are a novel class of homeostatic lipid mediators that protect neural cell integrity upon injury. Sci Adv 3, e1700735. doi:10.1126/sciadv.1700735.

Blasiak, B., Barnes, S., Foniok, T., Rushforth, D., Matyas, J., Ponjevic, D., et al. (2013). Comparison of T2 and T2*-weighted MR molecular imaging of a mouse model of glioma. BMC Med Imaging 13, 20. doi:10.1186/1471-2342-13-20.

Bouteille, B., Oukem, O., Bisser, S., and Dumas, M. (2003). Treatment perspectives for human African trypanosomiasis. Fundam Clin Pharmacol 17, 171-181. doi:10.1046/j.1472-8206.2003.00167.x.

Calandria, J. M., Sharp, M. W., and Bazan, N. G. (2015). The Docosanoid Neuroprotectin D1 Induces TH-Positive Neuronal Survival in a Cellular Model of Parkinson’s Disease. Cell. Mol. Neurobiol. 35, 1127-1136. doi:10.1007/s10571-015-0206-6.

Chen, C.-H., Chen, P.-Y., Lin, Y.-Y., Feng, L.-Y., Chen, S.-H., Chen, C.-Y., et al. (2019). Suppression of tumor growth via IGFBP3 depletion as a potential treatment in glioma. Journal of Neurosurgery 132, 168-179. doi:10.3171/2018.8.JNS181217.

Dumas, M., and Bisser, S. (1999). “Clinical aspects of human African trypanosomiasis,” in Progress in Human African Trypanosomiasis, Sleeping Sickness, eds. M. Dumas, B. Bouteille, and A. Buguet (Paris: Springer), 215-233. doi:10.1007/978-2-8178-0857-4_13.

Esquenazi, S., He, J., Bazan, H. E. P., and Bazan, N. G. (2004). Prevention of experimental diffuse lamellar keratitis using a novel platelet-activating factor receptor antagonist. JCataract Refract Surg 30, 884-891. doi:10.1016/j.jcrs.2003.09.069.

Esquenazi, S., He, J., Li, N., Bazan, N. G., Esquenazi, I., and Bazan, H. E. P. (2009). A novel platelet activating factor receptor antagonist reduces cell infiltration and expression of inflammatory mediators in mice exposed to desiccating conditions after PRK. Clin. Dev. Immunol. 2009, 138513. doi:10.1155/2009/138513.

Florman, J. E., Evans, D. L., Lu-Emerson, C., Cushing, D., and Rughani, A. (2016). Survival With Glioblastoma Multiforme and the Timing of Chemoradiation. Neurosurgery 78, E313-E314. doi:10.1227/NEU.0000000000001077.

Han, S. J., Rutledge, W. C., Molinaro, A. M., Chang, S. M., Clarke, J. L., Prados, M. D., et al. (2015). The Effect of Timing of Concurrent Chemoradiation in Patients With Newly Diagnosed Glioblastoma. Neurosurgery 77, 248-253. doi:10.1227/NEU.0000000000000766.

Harvey, L. D., Yin, Y., Attarwala, I. Y., Begum, G., Deng, J., Yan, H. Q., et al. (2015). Administration of DHA Reduces Endoplasmic Reticulum Stress-Associated Inflammation and Alters Microglial or Macrophage Activation in Traumatic Brain Injury: ASN Neuro. doi:10.1177/1759091415618969.

He, J., and Bazan, H. E. P. (2006). Synergistic effect of platelet-activating factor and tumor necrosis factor-alpha on corneal myofibroblast apoptosis. Invest. Ophthalmol. Vis. Sci. 47, 883-891. doi:10.1167/iovs.05-0581.

Jeffes, E. W. B., Zhang, J. G., Hoa, N., Petkar, A., Delgado, C., Chong, S., et al. (2005). Antiangiogenic Drugs Synergize with a Membrane Macrophage Colony-Stimulating Factor-Based Tumor Vaccine to Therapeutically Treat Rats with an Established Malignant Intracranial Glioma. The Journal of Immunology 174, 2533-2543. doi:10.4049/jimmunol.174.5.2533.

Kappagoda, S., Singh, U., and Blackburn, B. G. (2011). Antiparasitic Therapy. Mayo Clin Proc 86, 561-583. doi:10.4065/mcp.2011.0203.

Marrero, L., Wyczechowska, D., Musto, A. E., Wilk, A., Vashistha, H., Zapata, A., et al. (2014a). Therapeutic efficacy of aldoxorubicin in an intracranial xenograft mouse model of human glioblastoma. Neoplasia 16, 874-882. doi:10.1016/j.neo.2014.08.015.

Marrero, L., Wyczechowska, D., Musto, A. E., Wilk, A., Vashistha, H., Zapata, A., et al. (2014b). Therapeutic efficacy of aldoxorubicin in an intracranial xenograft mouse model of human glioblastoma. Neoplasia 16, 874-882. doi:10.1016/j.neo.2014.08.015.

Mooney, J., Bernstock, J. D., Ilyas, A., Ibrahim, A., Yamashita, D., Markert, J. M., et al. (2019). Current Approaches and Challenges in the Molecular Therapeutic Targeting of Glioblastoma. World Neurosurgery 129, 90-100. doi:10.1016/j.wneu.2019.05.205.

Mukherji, S. K. (2010). Bevacizumab (Avastin). American Journal of Neuroradiology 31, 235-236. doi:10.3174/ajnr.A1987.

Ozdemir-Kaynak, E., Qutub, A. A., and Yesil-Celiktas, O. (2018). Advances in Glioblastoma Multiforme Treatment: New Models for Nanoparticle Therapy. Front. Physiol. 9. doi:10.3389/fphys.2018.00170.

Soda, Y., Myskiw, C., Rommel, A., and Verma, I. M. (2013). Mechanisms of neovascularization and resistance to anti-angiogenic therapies in glioblastoma multiforme. J. Mol. Med. 91, 439-448. doi:10.1007/s00109-013-1019-z.

von Neubeck, C., Seidlitz, A., Kitzler, H. H., Beuthien-Baumann, B., and Krause, M. (2015). Glioblastoma multiforme: emerging treatments and stratification markers beyond new drugs. Br J Radiol 88. doi:10.1259/bjr.20150354.

Emiliano S. Lopez, Manglio M. Rizzo, J Oscar Croxatto, Guillermo Mazzolini, Juan E. Gallo. Suramab, a Novel Antiangiogenic Agent, Reduces Tumor Growth and Corneal Neovascularization. Cancer Chemother Pharmacol 2011 Mar;67(3):723-8.

Emiliano S. Lopez, Gustavo A. Ortiz, Constanza Potilinski, J Oscar Croxatto, Juan E. Gallo. Corneal Neovascularization: A Combined Approach of Bevacizumab and Suramin Showed Increased Antiangiogenic Effect Through Downregulation of BFGF and P ₂ × ₂. Curr Eye Res 2018 Apr;43(4):466-473

Daniela A. Quinteros, Emiliano S. Lopez, Juan L. Silva Couto, Belkys A. Maletto, Daniel A. Allemandi, Santiago D. Palma, Juan E. Gallo. Evaluation of the Performance of an Ophthalmic Thermosensitive Hydrogel Containing Combination of Suramin and Bevacizumab. Curr Pharm Des 2016;22(43):6587-6594.

### Example 3

Introduction: Glioblastoma Multiforme (GBM) is the most aggressive primary brain tumor with a poor prognosis and average survival of 12-14 months with the current standard of treatment: surgical resection, radiation, and chemotherapy. The inefficacy of current treatments necessitates the development of new GBM therapeutics.

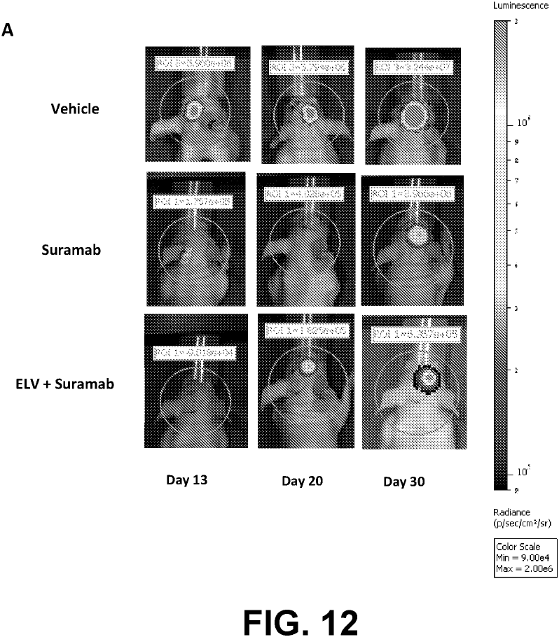

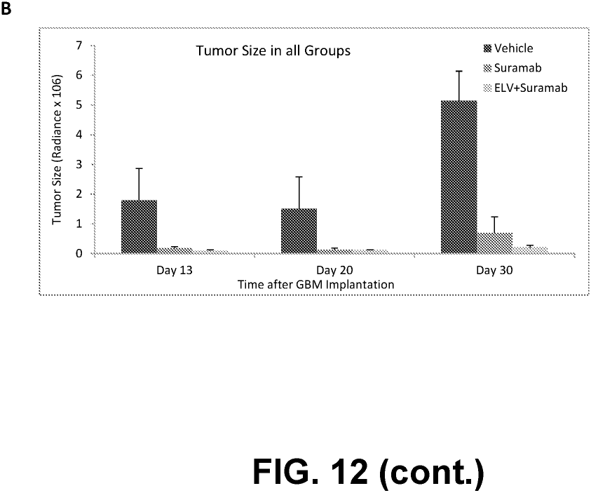

Methods: Female athymic nude mice were anesthetized with ketamine/xylazine and physiologically monitored. A human GBM cell line was sterotactically implanted into the right striatum. On day 13 following implantation mice received one of the following treatments via intraperitoneal injection: Saline; Suramab, ELV + Suramab. Bioluminescent imaging was performed on days 13 (before treatment), 20, and 30 days post implantation, representative images shown in FIG. 12 panel A. On day 30 mice were perfused and ex vivo MRI was conducted.

Results: No adverse physiologic effects were observed in Suramab and ELV + Suramab treatment groups. Growth imaging scores were reduced by Suramab and ELV + Suramab compared to vehicle. On day 30 vehicle animals had a score of 10.6 × 10⁶ compared to Suramab and ELV+ Suramab animals with scores of 0.69 × 10⁶ and 0.20 × 10⁶ respectively (FIG. 12 panel B).

Conclusion: Tumor size was dramatically reduced by Suramab (94%) and ELV+ Suramab (98%) on day 30 compared to the vehicle. The results demonstrate the therapeutic usefulness of these new compounds when applied to a GBM model.

### Example 4

### Multiprong Control of Glioblastoma Multiforme Invasiveness: Blockade of Pro-Inflammatory Signaling, Anti-Angiogenesis and Homeostasis Restoration

### Abstract

Glioblastoma multiforme (GBM) is the most invasive type of glial tumor with poor overall survival, despite advances in surgical resection, chemotherapy, and radiation. One of the main challenges in treating GBM is related to the tumor’s location, complex and heterogeneous biology, and high invasiveness. To meet the demand for oxygen and nutrients, growing tumors induce new blood vessels growth. Antibodies directed against vascular endothelial growth factor (VEGF), which promotes angiogenesis, have been developed to limit tumor growth. Bevacizumab (Avastin), an anti-VEGF monoclonal antibody, is the first approved angiogenesis inhibitor with therapeutic promise. However, it has limited efficacy, which can be due to adaptive mutations in GBM, leading to overall survival compared to the standard of care in GBM patients. Molecular connections between angiogenesis, inflammation, oxidative stress pathways, and the development of gliomas have been recognized. Improvement in treatment outcomes for patients with GBM can require a multifaceted approach due to the converging dysregulation of signaling pathways. While some GBM clinical trials focus on “anti-angiogenic” modalities, stimulating inflammation resolution is a novel host-centric therapeutic avenue. The selective therapeutic possibilities for targeting the tumor microenvironment, specifically angiogenic and inflammatory pathways expand. So, a combination of agents aiming to interfere with several mechanisms can be beneficial to improve outcomes. Our approach can also be combined with other therapies to enhance sustained effectiveness. Here, we validate Suramab (anti-angiogenic), LAU-0901 (a platelet-activating factor receptor antagonist), Elovanoid (ELV; a new lipid mediator), and their combination as new embodiments to contain GBM growth and invasiveness.

### Introduction

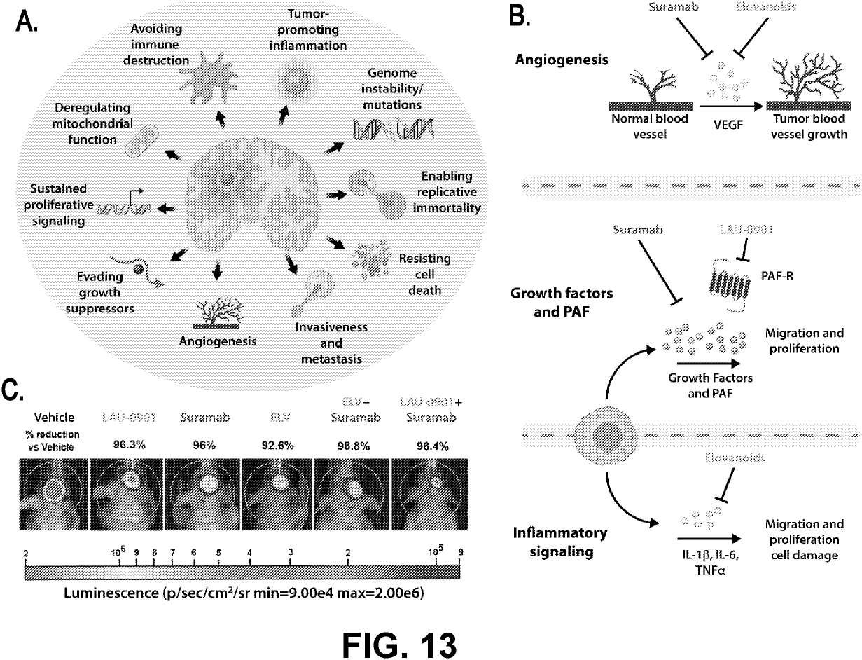

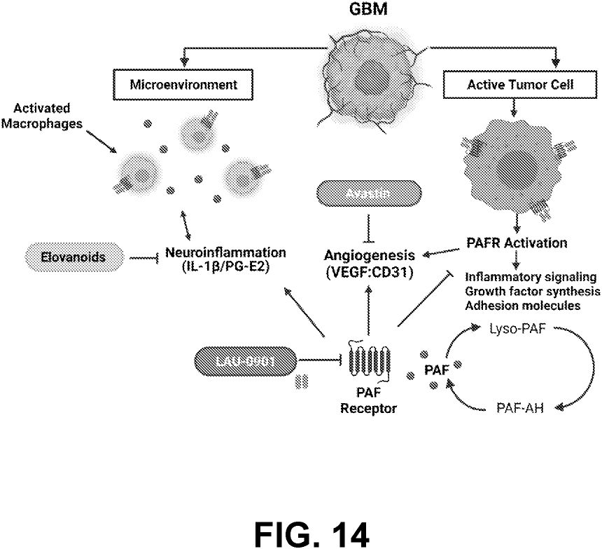

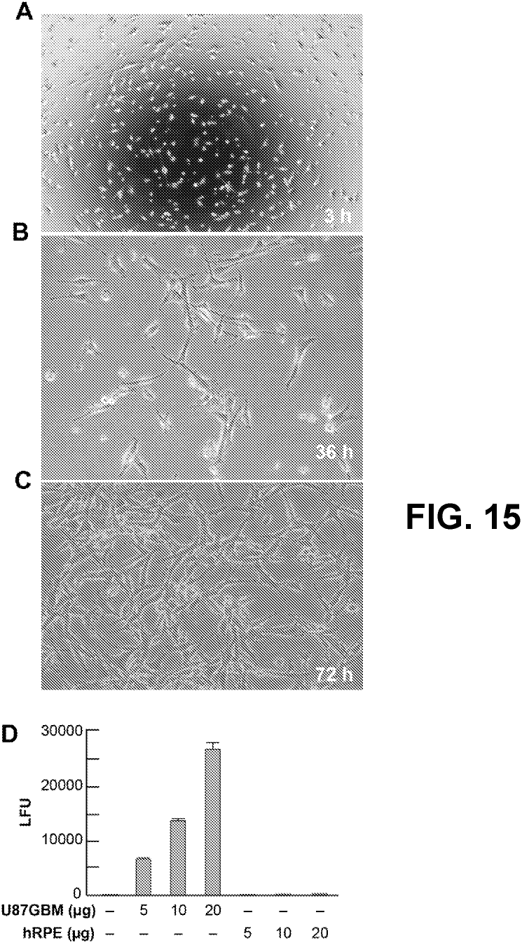

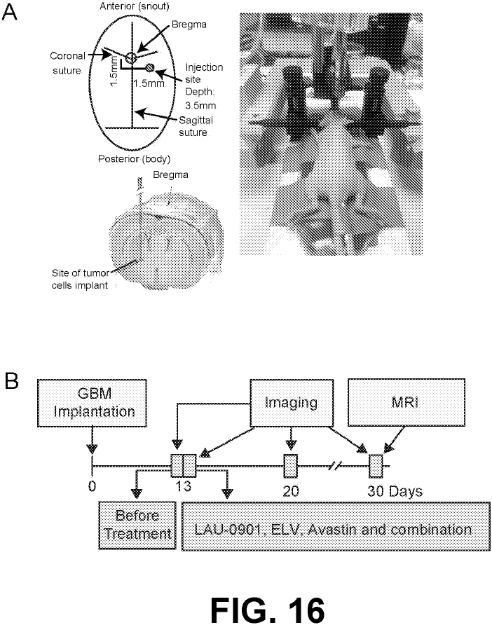

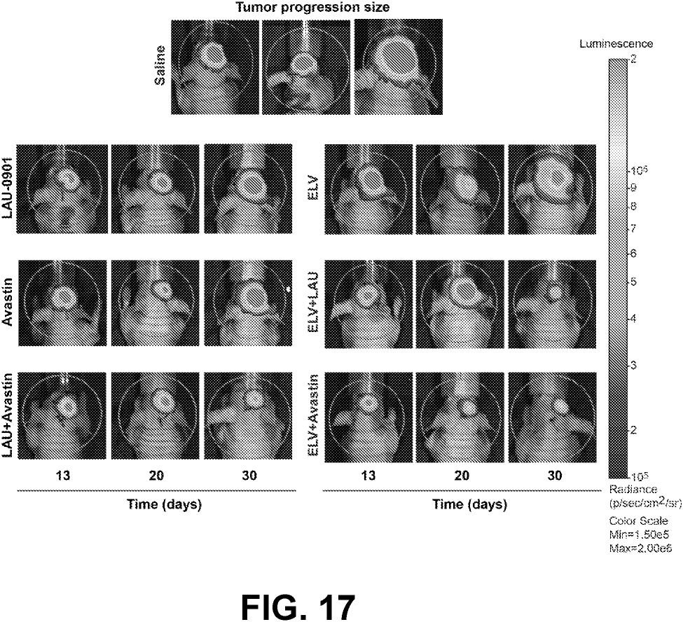

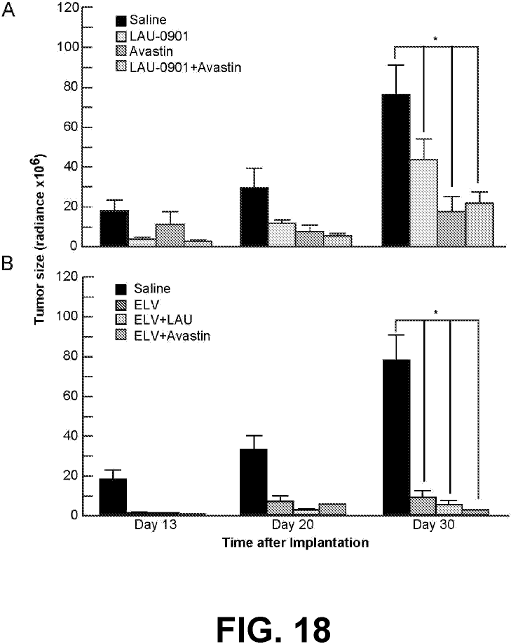

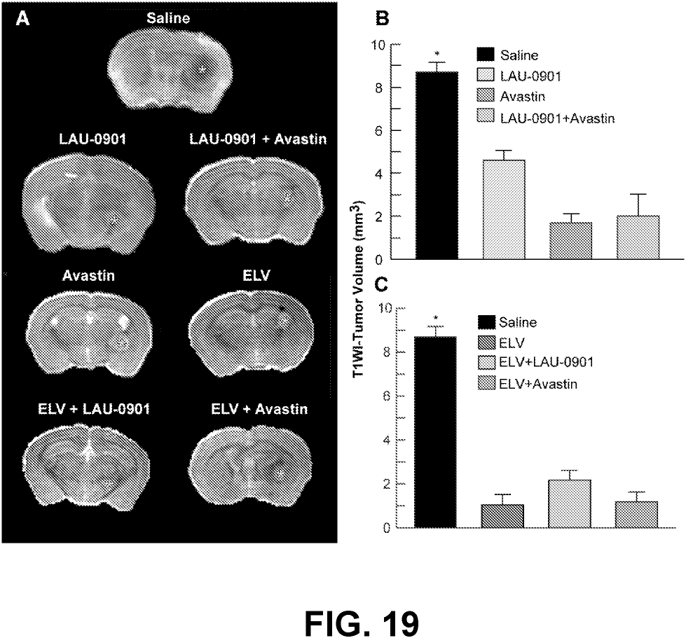

Glioblastoma multiforme (GBM) is the most common and lethal intracranial malignancy, with a few advances in treatment over several decades. Standard-of-care therapy includes aggressive resection, radiation, and chemotherapy, but the median overall survival remains less than two years [1]. One of the challenges in the treatment of GBM is its aggressive growth characteristics. Complete surgical resection of the tumor is impossible because of infiltrative growth, multiple lesions, and microscopic spread. Thus, there is a strong need for new and effective GBM treatment. The molecular heterogeneity of GBM allows for adaptive mutations and drug resistance; thus, a multi-target approach can be necessary targeting cells in the microenvironment. Non-transformed cells in this microhabitat are less susceptible to these adaptations, which can make them an ideal target. The microenvironment of glioblastoma harbor multiple cell types, which, without wishing to be bound by theory, make distinct contributions to tumor progression and invasion [2] (FIG. 13). These cells include, but are not limited to microglia, astrocytes, macrophages, pericytes, fibroblasts, and vascular cells. Gliomas are highly vascular tumors, and the endothelial cells, pericytes, and astrocytes that form the neurovascular unit function support tumor progression. In addition, microglia cells promote glioma migration and tumor growth [2]. Astrocytes can be converted into a reactive phenotype by the glioma microenvironment and secrete many factors that influence tumor growth [3]. The elements, pathways, and interactions provide a new perspective on the cell biology of brain tumors, which can generate a new treatment paradigm (FIG. 13).

### Suramab (Combination of Suramin Plus Avastin) Is an Anti-Angiogenic Combination for GBM Treatment

Anti-angiogenic strategies for treating high-grade gliomas have a solid biologic rationale since these tumors produce large amounts of vascular endothelial growth factor (VEGF) and are highly vascular. Due to their high metabolic demands, growing solid tumors depends on vascularization to provide nutrients and oxygen and disposal of embolic waste products. Because VEGF is a critical growth factor required for new blood vessel formation, anti-VEGF agents were developed to block tumor growth by inhibiting blood vessel formation [4]. However, bevacizumab (brand name Avastin), a humanized monoclonal antibody developed to neutralize human VEGF, failed to improve survival benefit as monotherapy but conferred a survival benefit only in combination with chemotherapy or immuno-therapy [4]. Without wishing to be bound by theory, the success of combined treatments is that Avastin “normalizes” the abnormal vasculature of tumors, resulting in improved delivery of concurrently administered anticancer drugs, as well as alleviation of hypoxia.

Suramab is a new pharmaceutical combination of two anti-angiogenic compounds, suramin, and Avastin, which showed a high synergistic effect when administered jointly [5]. Suramin is a 100-year-old drug used to treat African sleeping sickness caused by Trypanosoma brucei rhodesiense [6]. It is a multifunctional molecule with applications, from parasitic and viral diseases to cancer, snakebite, and autism. It has demonstrated anticancer activity by inhibiting the binding of multiple growth factors and glutamatergic synaptic transmission [6]. It has an extremely long half-life, but repetitive dosing can result in drug accumulation [7]. Remarkably, a pharmaceutical combination of suramin and Avastin, administrated at relatively low doses, has a tremendous anti-angiogenic effect, synergistic like, with greater intensity and longer duration than the effect produced by mono-doses of Avastin or suramin [5]. It was demonstrated that Suramab strongly reduced tumor growth in colorectal carcinoma in mice and reduced neovascularization in a rabbit model of corneal angiogenesis [5]. There are no studies to evaluate the efficacy of Suramab on GBM so far.

### LAU-0901 Is a Selective PAF-Receptor Antagonist and a Potent Inhibitor of Inflammation and Tumor Growth

In contrast to anti-angiogenic strategies, stimulation of inflammation resolution is a new host-focused alternative to complement current therapies. The tumor microenvironment, which can be orchestrated by inflammatory molecules, promotes such tumors’ proliferation, survival, and migration, and it seems logical to employ anti-inflammatory drugs.

Described almost 50 years ago, the phospholipid mediator platelet-activating factor (PAF) has been implicated in many pathologic processes. PAF is a potent mediator of inflammation involved in inflammatory diseases such as atherosclerosis, cardiovascular diseases, and cancer [8]. PAF induces robust systemic pro-inflammatory, pro-proliferative, and delayed immune-suppressive responses via the activation of PAF receptor (PAF-R) is implicated in various pathological conditions [8]. Recent studies demonstrated the implication of PAF in cancer growth and metastasis [8]. Circulating or cancer cells synthesize PAF and its presence in the tumor microenvironment. Inducible pathways that result in the development of tumor angiogenesis and metastasis can involve PAF binding on its receptor. Increased expression of tumoral PAF-R has been associated with invasiveness, increased tumor stages, tumor status, and poor prognosis in lung and esophageal squamous cell carcinoma [8]. Notably, patients who experienced decreased overall survival were found to have tumors expressing high levels of PAF-R compared to those with low tumor PAF-R expression [9]. [8]. Reduction of tumor burden improved murine host survival, and the augmented efficacy of therapeutic agents has been observed using pharmacologic PAF-R antagonists [9]. Multiple structurally different but specific PAF-R antagonists have been shown to exert effects against experimental tumors [9]; however, these agents have yet to be explored in clinical settings. Thus, PAF can represent a rational therapeutic target in GBM. As a new PAF-R antagonist, LAU-0901 has been previously shown to be neuroprotective in inflammation and ischemic stroke models [10]. LAU-0901 (2,4,6-trimethyl-1, 4-dihydro-pyridine-3, 5-dicarboxylic acid) is a selective PAF-R antagonist and a potent inhibitor of inflammation response and apoptosis [10]. It has also been shown to exhibit neuroprotective bioactivity when applied to a model of focal cerebral ischemia in rats and mice [11].

### Elovanoids Are New Class of Lipid Mediators That Regulate Homeostasis

Tumor growth is angiogenesis-dependent, and enhanced inflammation is a risk factor for many cancers. Inflammation is regulated by endogenous specialized pro-resolving lipid-autacoid mediators (SPMs). This includes resolvins, lipoxins, and protectins, which inhibit angiogenesis and mediate endogenous resolution by stimulating macrophage phagocytosis of cellular debris, resulting in reduced localized inflammatory cytokines [12, 13]. Unlike the majority of anti-inflammatory agents, SPMs are non-immunosuppressive and non-toxic. It was demonstrated that pro-resolving lipid mediators and anti-angiogenic therapy exhibit synergistic anti-tumor activity via resolvin receptor activation [14]. Notably, resolvins (RvD4 or RvD5) inhibited tumor growth at doses 10,000 times lower than anti-inflammatory agents such as aspirin and NSAIDs [14]. We discovered ELV, a new class of endogenous pro-homeostatic lipid mediators that protect against excitotoxicity [15]. They are stereoselective mediators that can be made on-demand and can be derived from very long-chain polyunsaturated fatty acids and have been shown to have a potent ability to inactivate pro-apoptotic and pro-inflammatory signaling in experimental stroke and neurodegenerative diseases [15].

In addition to anti-angiogenic and anti-inflammatory pathways, free fatty acid oxidation has been closely linked to GBM. Enhanced fatty acid oxidation provides glioblastoma cells metabolic plasticity to accommodate its dynamic nutrient microenvironment [16]. Thus, dynamic metabolic reprogramming plays a vital role during glioma genesis, which allows for the adaptation, survival, and proliferation of these cells in the diverse microenvironment implicit in this tumor. Thus, inhibition of fatty acid oxidation can provide an indirect approach to reduce tumor growth.

The development of effective GBM therapy presents challenges, one of which is the molecular heterogeneity and genetic instability of these tumors. To overcome this complexity, a multipronged approach that targets key signaling pathways, specifically angiogenesis, inflammation, and oxidative stress pathways, can open new therapeutic avenues.

### References Cited in This Example

1. Tan, A. C., Ashley, D. M., López, G. Y., Malinzak, M., Friedman, H. S., & Khasraw, M. (2020). Management of glioblastoma: State of the art and future directions. CA: A Cancer Journal for Clinicians, 70(4), 299-312. https://doi.org/10.3322/caac.21613

2. Charles, N. A., Holland, E. C., Gilbertson, R., Glass, R., & Kettenmann, H. (2011). The brain tumor microenvironment. Glia, 59(8), 1169-1180. https://doi.org/10.1002/glia.21136

3. Fishbein, A., Hammock, B. D., Serhan, C. N., & Panigrahy, D. (2021). Carcinogenesis: Failure of resolution of inflammation? Pharmacology & Therapeutics, 218, 107670. https://doi.org/10.1016/j.pharmthera.2020.107670

4. Garcia, J., Hurwitz, H. I., Sandler, A. B., Miles, D., Coleman, R. L., Deurloo, R., & Chinot, O. L. (2020). Bevacizumab (Avastin®) in cancer treatment: A review of 15 years of clinical experience and future outlook. Cancer Treatment Reviews, 86, 102017. https://doi.org/10.1016/j.ctrv.2020.102017

5. Lopez, E. S., Rizzo, M. M., Croxatto, J. O., Mazzolini, G., & Gallo, J. E. (2011). Suramab, a novel anti-angiogenic agent, reduces tumor growth and corneal neovascularization. Cancer Chemotherapy and Pharmacology, 67(3), 723-728. https://doi.org/10.1007/s00280-010-1457-z

6. Wiedemar, N., Hauser, D. A., & Mäser, P. (2020). 100 Years of Suramin. Antimicrobial Agents and Chemotherapy, 64(3), e01168-19. https://doi.org/10.1128/AAC.01168-19

7. Grossman, S. A., Phuphanich, S., Lesser, G., Rozental, J., Grochow, L. B., Fisher, J., & Piantadosi, S. (2001). Toxicity, Efficacy, and Pharmacology of Suramin in Adults With Recurrent High-Grade Gliomas. Journal of Clinical Oncology, 19(13), 3260-3266. https://doi.org/10.1200/JCO.2001.19.13.3260

8. Tsoupras, A. B., Iatrou, C., Frangia, C., & Demopoulos, C. A. (2009). The implication of platelet activating factor in cancer growth and metastasis: potent beneficial role of PAF-inhibitors and antioxidants. Infectious Disorders Drug Targets, 9(4), 390-399. https://doi.org/10.2174/187152609788922555

9. de Oliveira, S. I., Andrade, L. N., Onuchic, A. C., Nonogaki, S., Fernandes, P. D., Pinheiro, M. C., Jancar, S. (2010). Platelet-activating factor receptor (PAF-R)-dependent pathways control tumour growth and tumour response to chemotherapy. BMC Cancer, 10(1), 200. https://doi.org/10.1186/1471-2407-10-200

10. He, J., & Bazan, H. E. P. (2006). Synergistic effect of platelet-activating factor and tumor necrosis factor-alpha on corneal myofibroblast apoptosis. Investigative Ophthalmology & Visual Science, 47(3), 883-891. https://doi.org/10.1167/iovs.05-0581

11. Belayev, L., Khoutorova, L., Atkins, K., Gordon, W. C., Alvarez-Builla, J., & Bazan, N. G. (2008). LAU-0901, a novel platelet-activating factor antagonist, is highly neuroprotective in cerebral ischemia. Experimental Neurology, 214(2), 253-258. https://doi.org/10.1016/j.expneurol.2008.08.009

12. Bazan, N. G., Eady, T. N., Khoutorova, L., Atkins, K. D., Hong, S., Lu, Y., Belayev, L. (2012). Novel aspirin-triggered neuroprotectin D1 attenuates cerebral ischemic injury after experimental stroke. Experimental Neurology, 236(1), 122-130. https://doi.org/10.1016/j.expneurol.2012.04.007

13. Panigrahy, D., Gilligan, M. M., Huang, S., Gartung, A., Cortés-Puch, I., Sime, P. J., Hammock, B. D. (2020). Inflammation resolution: a dual-pronged approach to averting cytokine storms in COVID-19? Cancer and Metastasis Reviews, 39(2), 337-340. https://doi.org/10.1007/s10555-020-09889-4

14. Sulciner, M. L., Serhan, C. N., Gilligan, M. M., Mudge, D. K., Chang, J., Gartung, A., Panigrahy, D. (2017). Resolvins suppress tumor growth and enhance cancer therapy. Journal of Experimental Medicine, 215(1), 115-140. https://doi.org/10.1084/jem.20170681

15. Bhattacharjee, S., Jun, B., Belayev, L., Heap, J., Kautzmann, M.-A., Obenaus, A., Bazan, N. G. (2017). Elovanoids are a novel class of homeostatic lipid mediators that protect neural cell integrity upon injury. Science Advances, 3(9). https://doi.org/10.1126/sciadv.1700735

16. Kant, S., Kesarwani, P., Prabhu, A., Graham, S. F., Buelow, K. L., Nakano, I., & Chinnaiyan, P. (2020). Enhanced fatty acid oxidation provides glioblastoma cells metabolic plasticity to accommodate to its dynamic nutrient microenvironment. Cell Death & Disease, 11(4), 1-13. https://doi.org/10.1038/s41419-020-2449-5

### Example 5

### Combined Therapy With Avastin, a PAF Receptor Antagonist and a Lipid Mediator Inhibited Glioblastoma Tumor Growth

### Abstract

Glioblastoma multiforme (GBM) is an aggressive, highly proliferative, invasive brain tumor with a poor prognosis and low survival rate. The current standard of care for GBM is chemotherapy combined with radiation following surgical intervention, altogether with limited efficacy, since survival averages 18 months. Improvement in treatment outcomes for patients with GBM requires a multifaceted approach due to the dysregulation of numerous signaling pathways. Recently emerging therapies to precisely modulate tumor angiogenesis, inflammation, and oxidative stress are gaining attention as potential options to combat GBM. Using a mouse model of GBM, we validate Avastin (suppressor of vascular endothelial growth factor and anti-angiogenetic treatment), LAU-0901 (a platelet-activating factor receptor antagonist that blocks pro-inflammatory signaling), Elovanoid (ELV), a new pro-homeostatic lipid mediator that protects neural cell integrity and their combination as an alternative treatment for GBM. Female athymic nude mice were anesthetized with ketamine/xylazine, and luciferase-modified U87MG tumor cells were stereotactically injected into the right striatum. On post-implantation day 13, mice received one of the following: LAU-0901, ELV, Avastin, and all three compounds in combination. Bioluminescent imaging (BLI) was performed on days 13, 20, and 30 post-implantation. Mice were perfused for ex vivo MRI on day 30. Bioluminescent intracranial tumor growth percentage was reduced by treatments with LAU-0901 (43%), Avastin (77%), or ELV (86%), individually, by day 30 compared to saline treatment. In combination, LAU-0901/Avastin, ELV/LAU-0901, or ELV/Avastin had a synergistic effect in decreasing tumor growth by 72%, 92%, and 96%, respectively. Additionally, tumor reduction was confirmed by MRI on day 30, which shows a decrease in tumor volume by treatments with LAU-0901 (37%), Avastin (67%), or ELV (81.5%), individually, by day 30 compared to saline treatment. In combination, LAU-0901/Avastin, ELV/LAU-0901, or ELV/Avastin had a synergistic effect in decreasing tumor growth by 69%, 78.7%, and 88.6%, respectively. We concluded that LAU-0901 and ELV combined with Avastin exert a better inhibitive effect in GBM progression than monotherapy. This study demonstrates the efficacy of these new therapeutic regimens in a model of GBM and validate their use as therapeutics in GBM patients.

### Introduction

Glioblastoma multiforme (GBM) is a high-grade tumor from glial cells of the central nervous system (CNS), accounting for 49% of malignant brain tumors (Chen et al., 2019; Ostrom et al., 2020). The current standard of care for GBM involves maximal safe surgical resection, radiation, and adjuvant chemotherapy. This conventional approach has shown little impact on the survival and prognosis for patients with GBM due to the heterogeneous, highly proliferative, and invasive nature of GBM (Stupp et al., 2005; Soda et al., 2013; von Neubeck et al., 2015; Mooney et al., 2019). Other strategies that aim to inhibit tumor angiogenesis can lead to an adaptive tumor response, transitioning to a more invasive phenotype (Bergers and Hanahan, 2008). Therefore, an elusive goal in brain cancer therapy is to develop targeted approaches against tumorigenic pathways that can lead to long-term, positive outcomes (Woodworth et al., 2014). Recently, emerging mediators to modulate tumor angiogenesis (Avastin), inflammation (LAU-0901), and oxidative stress (Elovanoids) are gaining attention as therapeutics to combat GBM (FIG. 14).

Avastin is a monoclonal antibody against vascular endothelial growth factor (VEGF) approved in 2008 to treat GBM. It has shown a radiographic response rate of up to 40% as a single agent or combined with chemotherapy for GBM recurrence (Mukherji, 2010). However, Avastin has limited efficacy, likely due to adaptive mutations in GBM (Bergers and Hanahan, 2008), leading to no improvement in overall survival compared to standard of care plus radiation in GBM patients (Mukherji, 2010; Ozdemir-Kaynak et al., 2018). Given the inefficacy of available therapeutics for GBM and its high incidence of recurrence, there is a critical need to develop therapies with a higher success rate.

Antagonizing platelet-activating factor (PAF) may be a rational, multipronged therapy for GBM. PAF is a potent pro-inflammatory lipid mediator that can be involved in the development of cancer and other inflammatory conditions. It is synthesized in circulating and cancer cells and secreted into the tumor microenvironment. PAF has been shown to enhance the production of growth factors, adhesion molecules, and cytokines that have been shown to play a role in tumor angiogenesis and metastasis (Tsoupras et al., 2009; Lordan et al., 2019). Thus, inhibition of PAF biosynthesis can provide an indirect approach to mitigating metastatic angiogenesis of tumors. LAU-0901 (2,4,6-trimethyl-1, 4-dihydro-pyridine-3, 5-dicarboxylic acid) is a highly selective PAF receptor (PAFR) antagonist and a potent inhibitor of apoptosis and inflammatory responses (Bazan et al., 1994; Bazan, 2003; He and Bazan, 2006; Musto et al., 2016; Belayev et al., 2020). It is highly protective when used as an anti-inflammatory in various models (Esquenazi et al., 2004, 2009; He and Bazan, 2006). It has also been shown to have neuroprotective bioactivity when applied to a model of ischemia-reperfusion injury in rats and mice (Belayev et al., 2008, 2009, 2012, 2020).

In addition to anti-inflammatories, mediators of oxidative stress have been closely linked to GBM (Conti et al., 2010). Molecular connections between inflammation, oxidative stress pathways, and the development of gliomas have been established (Alghamri et al., 2021). The tumor microenvironment, which can be orchestrated by inflammatory molecules, promotes the proliferation, survival, and migration of such tumors. We characterized a new class of lipid mediators termed Elovanoids (ELVs; ELV-N32 and ELV-N34), which are dihydroxylated derivatives of 32:6n3 and 34:6n3, respectively (Bhattacharjee et al., 2017). ELVs are stereoselective mediators which can be made on-demand and can be derived from very long-chain polyunsaturated fatty acids (VLC-PUFAs) (Calandria et al., 2015). They are a new class of endogenous pro-homeostatic lipid mediators that protect against excitotoxicity and cell damage and modulate inflammatory responses (Bhattacharjee et al., 2017; Bazan, 2018). We demonstrated that ELV-N34:6 resulted in reduced infarct volumes, promoted cell survival, and diminished neurovascular unit disruption when administered after experimental focal cerebral ischemia (Bhattacharjee et al., 2017). Without wishing to be bound by theory, ELVs can have a protective effect on neural environments under metabolic catastrophe caused by GBM.

We validate the effect of LAU-0901, ELV-N34:6, and Avastin individually and all three compounds in combination to mitigate GBM. Our treatments were compared relative to individual administration of Avastin, which is an approved medication to treat GBM. Here we compare the efficacy of these new therapies in an orthotopic model of GBM. Without wishing to be bound by theory, the combinatorial application of these agents can improve survival and limit tumor growth in an orthotopic model of GBM. Due to the complex interplay of multiple tumorigenic cascades involved in the dynamics of GBM progression and invasiveness, a combinatorial approach with the treatments investigated in this study can shed light on improving therapy and prognosis of GBM patients.

### Materials and Methods

### Animals and Ethics Statement

Studies were performed according to the National Institutes of Health guidelines and under nationally accepted principles in the care and use of experimental animals. The Institutional Animal Care and Use Committee (IACUC) at the Louisiana State University Health Sciences Center, New Orleans, approved the animal protocols used in this study. Athymic nude female mice (Charles Rivers Laboratories), 6 to 8 weeks of age, were used in all experiments. Water and food were available for ad libitum consumption. All efforts were made to minimize pain and suffering and reduce the number of mice used in these experiments.

### U87MG Cell Line With Luciferase Reporter

The human cell line U87 MG-Red-Flug (U87MG) containing a luciferase-expressing gene was purchased from PerkinElmer (Waltham, MA). Immediately after arrival, cells were stored in liquid nitrogen at the vapor phase until ready to use. Cells were thawed and placed into T-25 mm flasks with Eagle’s MEM (ATCC Cat. No. 30-2003, Manassas, VA) containing 10% FBS (Hyclone, GE Health Care/Fisher Scientific Cat. No. SH300071, Waltham, MA) and puromycin (2 µg/ml). Cells were allowed to grow for up to 72 hours at 37° C. before sub-culturing them in the same medium. Cell growth was monitored and photomicrographed at 3, 36, and 72 hours (FIG. 15 Panels A-C). 500,000 U87MG and human retinal pigment epithelial (hRPE) cells were allowed to grow separately in six-well plates for 72 hours at 37° C., to 80% confluency, in three separate experiments. Cell extracts were made and protein content was adjusted in µg/µL by the Bio-Rad method. Luciferase activity was measured in luciferase units (LFU) using a Glomax 20/20 luminometer in 5-20 µg protein extracts using Luciferin as substrate (FIG. 15 Panel D). hRPE cells were used in these experiments as controls to show the specificity of luciferase gene expression in the U87MG line.

### Orthotopic Model of GBM

Mice were anesthetized with a ketamine/xylazine cocktail solution (100 mg/kg; 10 mg/kg) and secured in a stereotactic head frame. A midline, 1 cm incision was made over the scalp. Natural tear lubricant was applied to the eyes. For each mouse, 5 × 10⁶ U87MG cells in 5 µL serum-free Dulbecco’s Modified Eagle Medium (DMEM) were injected into the right hippocampus using a 10 µL Hamilton syringe at the following coordinates related to the bregma: 1.5 mm lateral, 1.5 mm posterior, and 3.5 mm in depth. The needle was lowered to 3.5 mm and retracted by 1 mm, before injection (FIG. 16 Panel A) (Marrero et al., 2014). Instruments to control rectal (CMA/150 Temperature Controller, CMA/Microdialysis AB, Stockholm, Sweden) and cranial (temporalis muscle; Omega Engineering, Stamford, CT) temperatures were closely maintained at 36-37° C. before, during, and after the procedure. The incision was sutured using sterile black monofilament nylon 5.0, and the area was cleaned with betadine. Mice were individually caged, observed daily for body weight, temperature, and locomotor changes. Animals were perfused at the end of the 30-day survival and brains removed for ex vivo MRI. A non-limting, exemplary experimental design is presented in FIG. 16 Panel B.

### Treatments

Specific doses of LAU-0901 (30 mg/kg) and ELV 34:6 (5 µg), which have been shown to provide the best neuroprotection in the stroke model (Belayev et al., 2008; Bhattacharjee et al., 2017), and Avastin (10 mg/kg, 0.2 mg/mouse) in the same murine model of glioblastoma (Pechman et al., 2011) were therefore chosen for this study. Mice were randomly and blindly allocated to eight treatment groups. The following treatments were used (n=5-7 per group): 1) LAU-0901, 30 mg/kg, IP; daily x 5 days; 2) ELV 34:6, 30 µg/mouse, IP; once; 3) Avastin 0.2 mg/mouse, IP, weekly x 2 doses (Pechman et al., 2011); 4) Saline in equal volume (0.2 mL/mouse); 5) LAU-0901 + ELV; 6) LAU-0901 + Avastin; and 7) ELV 34:6 + Avastin. For combinatory treatment, LAU-0901 was administered first, followed by Avastin or ELV 5 minutes later. For ELV combinatory treatment, ELV was administered first, followed by Avastin 5 minutes later. Treatment was administered on post-implantation day 13, and the bioluminescent imaging (BLI) time course started.

### Bioluminescence Imaging (BLI)

In vivo intracranial tumor growth was quantified by BLI using a Xenogen IVIS200 biophotonic imager (Caliper) facilitated by the Morphology and Imaging Core of the LSU Health School of Medicine. Mice were randomly assigned to individual treatment groups. For each imaging session, mice were injected intraperitoneally with D-Luciferin (150 mg/kg) 5 minutes before imaging. Anesthesia was administered by isoflurane-oxygen mix (3%) in an XGI-8 system equipped with a vaporizer and induction chamber. Following induction, mice were moved to the IVIS200 imaging chamber equipped with a 5-position manifold and enough nose cones to simultaneously sustain and image groups of five mice. Tumor growth was measured on days 13, 20, and 30 post-implantation. Images were captured and quantified using Living Image 4.1 software based on equivalent regions of interest (ROI) over the head. Emitted radiance values are reported in photons/second, as previously described (Marrero et al., 2014).

### Magnetic Resonance Imaging and Data Analyses

High-resolution ex vivo MRI was performed on brains perfused with PBS and 4% paraformaldehyde (PFA) with 8 mM Gd-DTPA (gadobenate dimeglumine; 529 mg/ml; Henry Schein) on day 30. T1-weighted images (T1WI) were obtained on 11.7T Bruker Advance 8.9 cm horizontal bore instrument equipped with an 89 mm (ID) receiver coil (Bruker Biospin, Billerica, MA). We used the following parameters: TR/TE = 1000 / 7 ms, matrix = 1282, 25×0.5 mm slices, FOV=1.8 cm, NEX = 6. MRI acquisition time was ~9.5 min with an in-plane resolution of 234 um. Cheshire image processing software (Hayden Image/Processing Group, Waltham, MA) was used to manually outline the whole brain and tumor volumes enhanced by Gd deposition. T1WI data were optimized for signal intensity to enhance tumor visualization. Tumors were identified as hyperintense (T1WI) within the striatum and surrounding tissues. Whole brain and tumor volumes (mm³) were extracted and analyzed (Jeffes et al., 2005; Blasiak et al., 2013).

### Statistical Analysis

Data are presented as mean values ± SEM. Analysis of variance (ANOVA) with repeated measures, followed by Bonferroni procedures to correct for multiple comparisons, was used to compare groups. Two-tailed Student’s t-tests were used for two-group comparisons. P<0.05 was considered statistically significant.

### Results

### Animal Physiology

All animals in treatment groups survived apart from two-vehicle-treated animals. An increase in body weight in all groups except for the group individually dosed with LAU-0901 was observed. No significant changes were measured in rectal temperature in mice treated with LAU-0901 + Avastin, Avastin, ELV + LAU-0901, or ELV + Avastin during the 30 days of survival compared to vehicle. Treatment with ELV + Avastin increased body weight most significantly by ~17% on day 30 compared to all treatment groups. In contrast, animals that received vehicles did not experience a significant increase in body weight during the 30-day survival period.

### Evaluation of U87MG-Luc and BLI Assays

Representative images of cell cultures showing cell growth measured at 3, 36, and 72 hours (FIG. 15 Panels A-C) revealed steady growth and the morphological pattern of U87MG cells, expressing a luciferase reporter. The level of luciferase activity increased in U87MG cell protein extracts compared to hRPE (FIG. 15 Panel D). An hRPE cell line was used as a standard control in our assays since they do not contain the luciferase gene. Tumor growth was measured on days 13, 20, and 30 using in vivo biophotonic imaging. Representative images of tumor-bearing mice are presented in FIG. 17. The emitted radiance indicated the number of live cells and indicated tumor burden (FIG. 17). During the first 13 days, all intracranial tumors increased in size, with a significant difference seen only in ELV and ELV + Avastin treated groups with a p-value of p=0.04 and p=0.049 compared to vehicle. There was progressive and rapid tumor growth in the saline group. On day 20, all mice had intracranial tumors, which varied in size, although all treated mice appeared to exhibit smaller tumors than saline-treated mice. On day 30, two mice from the saline group were dead, and the remaining six had extensive tumors.

Quantification of BLI tumor growth over time is presented in FIG. 18 Panel A and Panel B. Tumor size was reduced by all treatments on day 20 but did not reach statistical significance from the vehicle group. In contrast, tumor size was significantly reduced on day 30 by the following percentages: LAU-0901 by 43%, Avastin by 77%, LAU-0901 + Avastin by 72%, ELV by 86%, ELV + LAU-0901 by 92%, and ELV + Avastin by 96% (FIG. 18 Panel A and Panel B). Tumor growth was lowest in the ELV + Avastin treatment group and showed the most significant reduction compared to vehicle (FIG. 18 Panel B).

### T1W1 Evaluation of Brain Tumors

T1WI revealed more extensive tumor growth in vehicle-treated animals but reduced growth in all animals that received experimental treatments (FIG. 19 Panel A). Tumor volume was reduced compared to the vehicle by 37% in animals treated with LAU-0901, 67% when treated with Avastin, and 69% when treated with LAU-0901 + Avastin (FIG. 19 Panel B). Further reduction in tumor volume was observed in groups administered with ELV. We measured an 81.5% reduction when treated with ELV and 78.7% when treated with ELV + LAU-0901. The smallest tumor volume on day 30 was observed in the group treated with ELV + Avastin and showed the most significant (p<0.001) reduction by 88.6% compared to vehicle (FIG. 19 Panel C).

### Discussion

We have shown here that LAU-0901 (PAF receptor antagonist), ELV (a lipid mediator), Avastin (monoclonal antibody against VEGF), and their combination improved survival and reduced tumor volume and growth in the experimental GBM model. Intracranial tumor reduction was confirmed by BLI on days 20 and 30 and by MRI on day 30.

GBM is the most aggressive and lethal malignancy of the CNS, with a poor prognosis and median survival of 8 months (Ostrom et al., 2020). Several treatments have been evaluated in patients with recurrent or progressive GBM without consistent survival benefit (Mooney et al., 2019). One pathologic feature of GBM that distinguishes it from lower-grade glial tumors is the extent of microvascular proliferation. The hypoxic environment of the GBM tumor core influences the sprouting of capillaries from preexisting blood vessels through the upregulation of hypoxia-inducible factor (HIF 1-alpha), which triggers the downstream transcription of VEGF. VEGFs activate endothelial cells by binding to VEGF receptor tyrosine kinases to stimulate the endothelial cell proliferation and permeability of vessels to support the metabolic demands of GBM. To that end, Avastin remains a characterized suppressor of VEGF-A and anti-angiogenetic treatment (Garcia et al., 2020). However, the efficacy of Avastin is limited by adaptive mutations in GBM (Bergers and Hanahan, 2008). As a result, numerous targeted approaches involving Avastin have been investigated, such as its combination with immune checkpoint inhibitors, with encouraging results for treating lung, renal cell, hepatocellular carcinomas, and PARP inhibitor patients with ovarian cancer (Huang et al., 2017; Garcia et al., 2020). Similar to the suppression of tumor growth in the mouse GBM model, the combination of Avastin with other chemotherapeutic agents has been proven effective against non-GBM neoplasm growth (Freitas and Campos, 2019). However, the application of therapies that show improved outcomes in GBM patients remains a challenge. The lack of a durable response can be attributed to the acquisition of chemoresistance due to the activation of pathways that enhance cell survival, angiogenesis, and invasion (Woodworth et al., 2014) to treat malignancies such as GBM.

This limitation can warrant additional investigation on multipronged approaches to target specific signaling pathways used by GBM to overcome conventional therapies. Therefore, new treatments that can prevent or overcome resistance mechanisms in GBM are needed. So far, no significant efficacy with therapeutic agents alone has been demonstrated (Yamada et al., 2020). Our study investigates the effect of LAU-0901, ELV-N34:6, Avastin and their combination that would significantly increase the probability of survival of mice with intracranial implantation of the luciferase-modified U87MG tumor cells as potential treatments for GBM. This study shows that individual or concurrent application of LAU-0901, ELV-N34:6, or Avastin can improve survival in the GBM mouse model.

Over-activation of PAFR has been shown to accelerate tumor cell proliferation and other pro-tumorigenic effects (da Silva-Junior et al., 2018). Recent studies indicate that PAF receptor-dependent mechanisms can be responsible for modifying the tumor microenvironment, including the phenotype of tumor macrophages (da Silva-Jr et al., 2017). Excess PAF, which occurs under pathologic conditions, can become neurotoxic, and inhibition of this process enhances neuronal survival (Bazan, 2005; Tian and Bazan, 2005). Thus, PAF can be a therapeutic target in GBM. As a PAFR antagonist, LAU-0901 has been shown to be neuroprotective in inflammation, epilepsy, and ischemic stroke models (Bazan et al., 1994; Bazan, 2005; Belayev et al., 2008, 2009, 2012; Musto et al., 2016). PAF is a potent phospholipid messenger and, when overproduced, acts as an inflammatory mediator that stimulates cell infiltration and expression of cyclooxygenase-2 (COX-2) (Tian and Bazan, 2005; Esquenazi et al., 2009). COX-2 is rapidly induced in response to tissue injury and disease states to mediate events associated with severe inflammatory processes such as lipopolysaccharides, excitotoxicity, cytokines, and growth factors (Qiu et al., 2017). It is upregulated in tumor cells in tissues and accompanied by elevated levels of Prostaglandin E2 and selective COX-2 inhibitors, which have been demonstrated efficacious at reducing proliferation and migration of the U87MG cell line (Qiu et al., 2017). We demonstrated that LAU-0901 inhibits PAF, which activates the COX-2 pathway when overloaded (He and Bazan, 2006; Belayev et al., 2008, 2012). The significance of COX-2 during pathophysiological conditions presents it as an appealing intervention for inflammatory diseases. Also, PAF accumulation produces CNS damage through intracellular Ca²⁺ overload, reduction in cerebral blood flow (CBF), disruption of the blood-brain barrier, and stimulation of leukocytes (Belayev et al., 2009, 2012). We established that LAU-0901 increased local CBF when administered 2 hours after focal cerebral ischemia (Belayev et al., 2008). We present evidence validating the application of LAU-0901 to the GBM model impaired tumor growth, indicating PAF and COX-2 as targets. Our study used BLI and MRI to assess in vivo the effects conferred by LAU-0901 alone or in combination with Avastin and ELV in the GBM model. BLI demonstrated that intracranial tumor growth was significantly reduced by treatment with LAU-0901 alone on days 20 and 30. In combination, LAU-0901/Avastin and LAU-0901/ELV had a synergistic effect in decreasing tumor growth by 71-92%. Moreover, tumor reduction was confirmed by MRI on day 30.

ELVs are the first bioactive chemical messengers made from omega-3, a very-long-chain polyunsaturated fatty acid (VLC-PUFAs, n-3) released in response to cell injury or when cells are confronted with adversities for survival (Bhattacharjee et al., 2017; Bazan, 2018). Among the omega-3 family, docosahexaenoic acid (DHA; 22:6n3) is the most abundant PUFA and serves as a precursor of enzymatically derived dihydroxylated derivatives known as docosanoids. These derivatives include potent neuroprotective mediators which can be made “on-demand” when disruptions to homeostasis are impending (Bazan et al., 2011; Serhan et al., 2015). DHA has been shown to reduce the size of tumors and enhance the positive effects of the chemotherapy drug cisplatin while limiting its harmful side effects (Wang et al., 2011). Studies have investigated the effect of DHA on GBM cells in cell culture. DHA was shown to exert an anti-tumor effect, and treatment may be responsible for regulating the malignancy of GBM through the esterification of membrane phospholipids, altering permeability and mobility (Ruan et al., 2019). The precursors of ELVs are made by elongation of DHA and catalyzed by ELOVL4 (elongation of very-long-chain fatty acids-4). ELVs counteract oxygen-glucose deprivation, N-methyl-D-aspartate (NMDA)-induced excitotoxicity, or MCAo-induced ischemic stroke (Bhattacharjee et al., 2017; Bazan, 2018). They are rapidly synthesized in the presence of homeostatic disruptors and when cells need to counteract neuroinflammatory responses to protect their integrity and prevent cell death (Bazan, 2018).

Inflammation-induced mutagenesis can be a hallmark of cancer due to the genetic instability it causes. Thus, regulation of inflammatory signaling in the tumor microenvironment can help mitigate the tumors ability to acquire adaptive mutations and resistance to therapy. Mutation rates in inflamed microenvironments have been shown to increase compared to normal tissue (Colotta et al., 2009; Grivennikov et al., 2010). Through modulation of inflammatory signaling by both LAU-0901 and ELV, they can contribute to reducing adaptive mutagenesis, preventing tumor cells from acquiring resistance to therapeutics, thus providing a possible mechanism of LAU-0901 and ELV bioactivity in glioblastoma. We discovered that treatment with ELVs in the mouse GBM model, both alone and in combination with LAU-0901 or Avastin, significantly reduced tumor growth and tumor size by day 30 by 71-92%. Tumor growth was lowest in the ELV + Avastin treatment group and showed the most significant reduction compared to vehicle-treated rats. ELVs greater inhibitory effect than the combination of LAU-0901 + Avastin can be attributed to its potent pro-homeostatic bioactivity targeting multiple signaling pathways. This can contribute to reducing cancer cell proliferation, survival, and migration that results from excessive inflammatory signaling in the microenvironment, inhibiting tumor growth in our model. This study demonstrates the efficacy of LAU-0901, ELV, and their combination in the experimental GBM model. We found that treatment with our new therapeutic approach reduced tumor volume and growth following xenograft implantation of human-derived GBM cells. The potentiation of Avastin by LAU-0901 and ELV offers a strategy that can improve clinical outcomes in patients with GBM.

### EQUIVALENTS

Those skilled in the art will recognize, or be able to ascertain, using no more than routine experimentation, numerous equivalents to the specific substances and procedures described herein. Such equivalents are considered to be within the scope of this invention, and are covered by the following claims.

What is claimed:

1. A method of treating or preventing cancer, the method comprising administering to a subject a two or more anti-cancer agents, wherein the two or more anti-cancer agents are selected from the group consisting of an elovanoid, a PAF-receptor antagonist; an anti-VEGF antibody; and Suramab.

2. A method of reducing tumor size, the method comprising administering to a subject a two or more anti-cancer agents, wherein the two or more anti-cancer agents are selected from the group consisting of an elovanoid, a PAF-receptor antagonist; an anti-VEGF antibody; and Suramab.

3. The method of claim 1 or claim 2, wherein the cancer or tumor comprises a solid tumor or a liquid cancer.

4. The method of claim 3, wherein the solid tumor comprises glioblastoma multiforme (GBM), colon cancer, prostate cancer or lung cancer.

5. The method of claim 1, wherein treating or preventing cancer is indicated by inhibiting or delaying tumor invasion, angiogenesis, tumor size, or any combination thereof.

6. The method of claim 1 or claim 2, wherein the two or more anti-cancer agents are administered sequentially, concurrently, or simultaneously.

7. The method of claim 1 or claim 2, wherein the two or more anti-cancer agents are administered systemically.

8. The method of claim 1 or claim 2, wherein the two or more anti-cancer agents are administered parenterally.

9. The method of claim 1 and claim 2, wherein the elovanoid comprises ELV-N32 and ELV-N34.

10. The method of claim 9, wherein ELV-N32 and ELV-N34 comprise 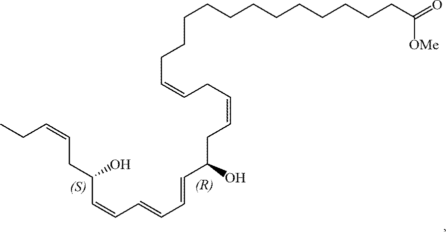 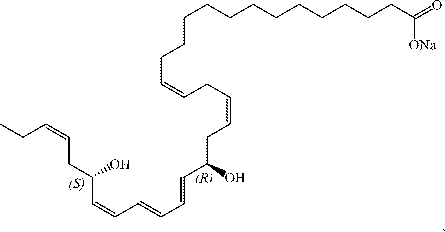 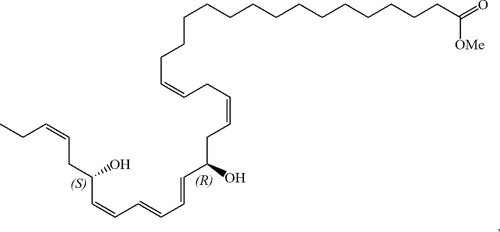 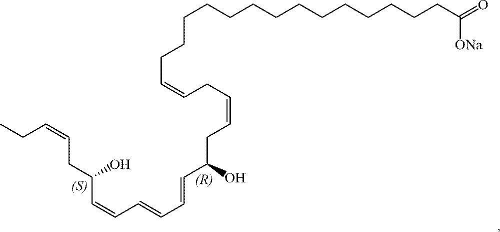 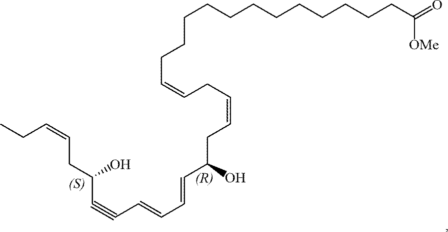 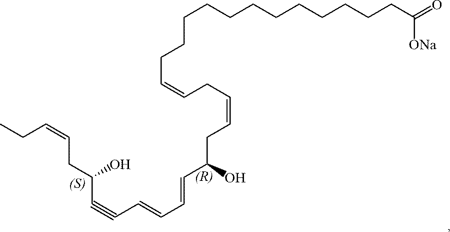 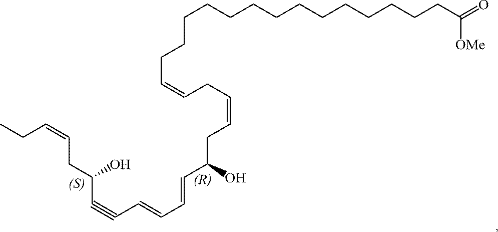 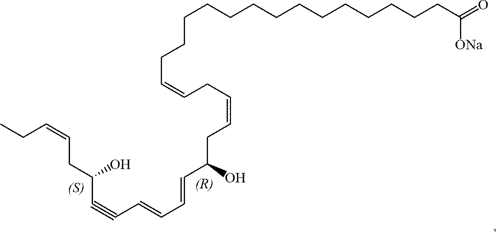
isomers thereof, or a combination thereof.

11. The method of claim 1 or claim 2, wherein the PAF-receptor antagonist comprises a compound according to Formula I:

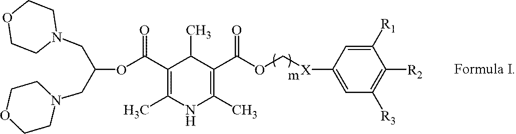

12. The method of claim 11, wherein m is 1 - 4; X is O or S; R₁ is H or Cl; R₃ is H or Cl; R₂ is H, butoxy, or Cl; and wherein, when: R₂ is butoxy, m is 1 or 4, or when R₁ and R₂ are both Cl, and X is O, m is 3 or 4.

13. The method of claim 11, wherein the compound of Formula I comprises 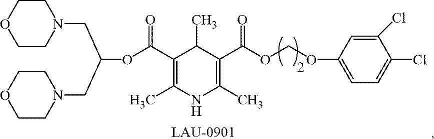 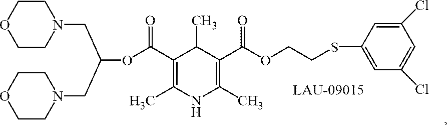 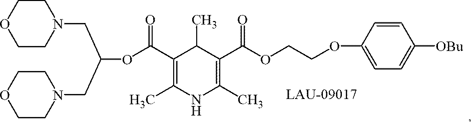 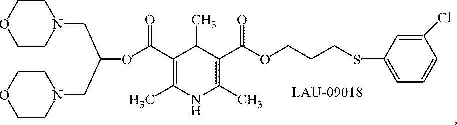 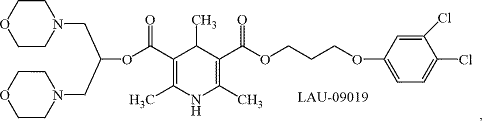 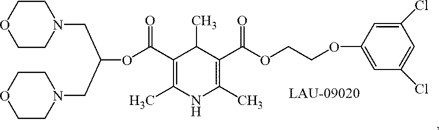 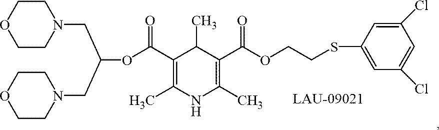 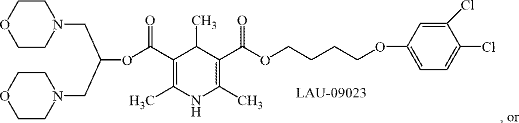 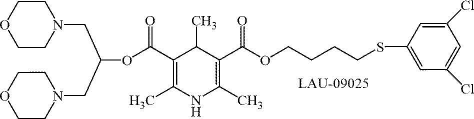
or a pharmaceutically acceptable salt thereof.

14. The method of claim 1 or claim 2, wherein the anti-VEGT antibody comprises bevacizumab.

15. An anti-cancer composition, wherein the anti-cancer composition comprises:
a. a therapeutically effective amount of an elovanoid and a therapeutically effective amount of a PAF-receptor antagonist;
b. a therapeutically effective amount of an elovanoid and a therapeutically effective amount of an anti-VEGF antibody;
c. a therapeutically effective amount of an elovanoid and a therapeutically effective amount of Suramab;
d. a therapeutically effective amount of a PAF-receptor antagonist and a therapeutically effective amount of an anti-VEGF antibody; or
e. a therapeutically effective amount of a PAF-receptor antagonist and a therapeutically effective amount of Suramab.

16. The anti-cancer composition of claim 15, wherein the elovanoid comprises ELV-N32 and ELV-N34.

17. The anti-cancer composition of claim 16, wherein ELV-N32 and ELV-N34 comprise 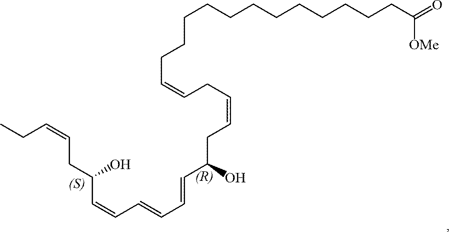 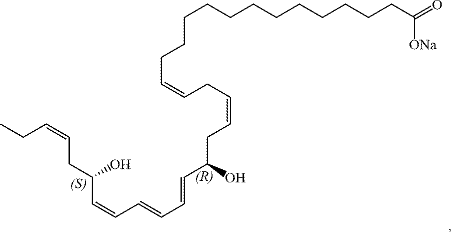 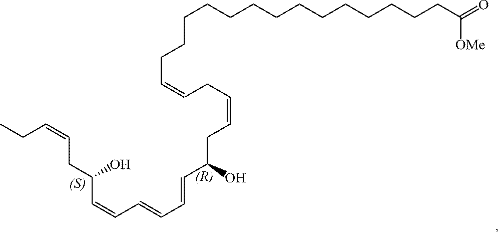 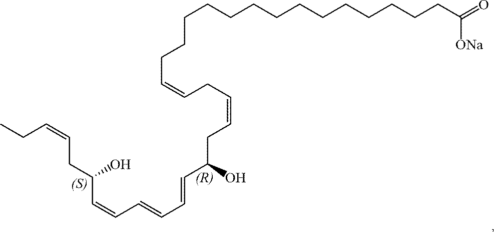 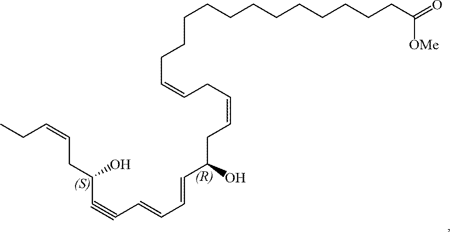 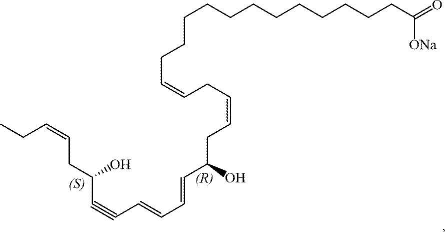 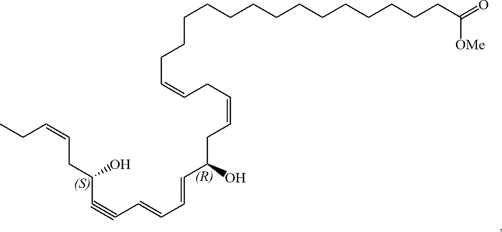 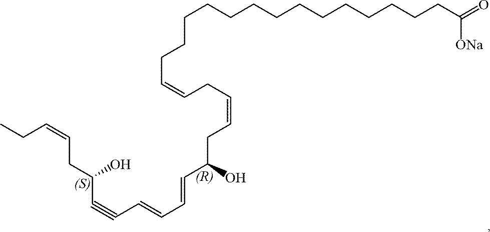
isomers thereof, or a combination thereof.

18. The anti-cancer composition of claim 15, wherein the PAF-receptor antagonist comprises a compound of Formula I: 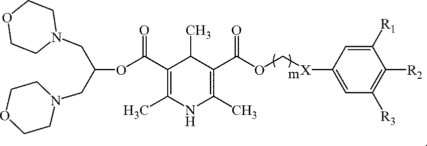
.

19. The anti-cancer composition of claim 18, wherein m is 1 - 4; X is O or S; R₁ is H or Cl; R₃ is H or Cl; R₂ is H, butoxy, or Cl; and wherein, when: R₂ is butoxy, m is 1 or 4, or when R₁ and R₂ are both Cl, and X is O, m is 3 or 4.

20. The anti-cancer composition of claim 18, wherein the compound of Formula I comprises 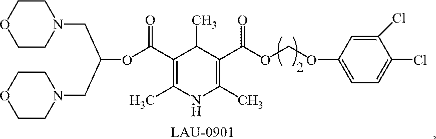 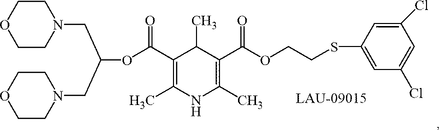 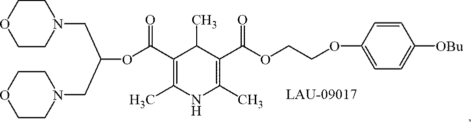 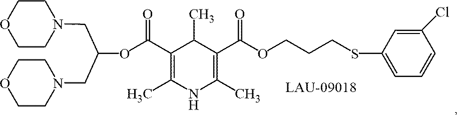 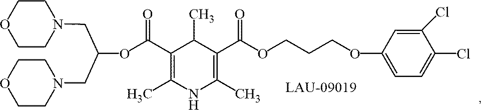 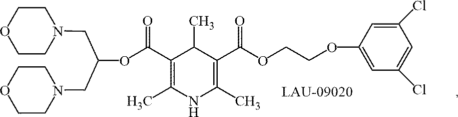 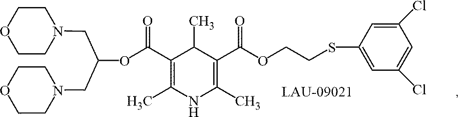 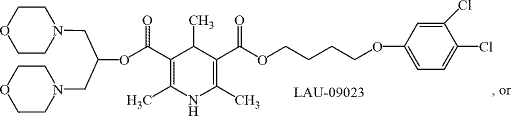 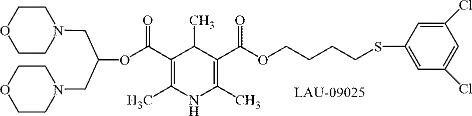
or a pharmaceutically acceptable salt thereof.

21. The anti-cancer composition of claim 15, wherein the anti-VEGF antibody comprises bevacizumab.

22. The anti-cancer composition of claim 15, wherein the anti-cancer composition is provided as a pharmaceutical composition.

23. The anti-cancer composition of claim 22, wherein the pharmaceutical composition further comprises an excipient, pharmaceutically acceptable carrier, or diluent.

24. A method of treating or preventing cancer, the method comprising administering to a subject a therapeutically effective amount of the composition of claim 15.

25. A method to reduce tumor size, the method comprising administering to a subject a therapeutically effective amount of the composition of claim 15.

26. The method of claim 24 or claim 25, wherein the cancer or tumor comprises a solid tumor or a liquid cancer.

27. The method of claim 26, wherein the solid tumor comprises glioblastoma multiforme (GBM), colon cancer, prostate cancer or lung cancer.

28. The method of claim 24, wherein treating cancer is indicated by inhibiting or delaying tumor invasion, angiogenesis, tumor size, or any combination thereof.

29. The method of claim 24 or claim 25, wherein the composition is administered systemically.

30. The method of claim 24 or claim 25, wherein the composition is administered parenterally.

31. The method of claim 30, wherein parenteral administration comprises injection.
